(19) United States
(12) Patent Application Publication
(10) Pub. No.: US 2005/0287132 A1
(43) Pub. Date: Dec. 29, 2005

(54) Biliverdin reductase fragments and variants, and methods of using biliverdin reductase and such fragments and variants

(75) Inventor: Mahin D. Maines, Rochester, NY (US)

Correspondence Address: Edwin V. Merkel;Nixon Peabody LLP, 1300 Clinton Square, P.O. Box 31051, Rochester, NY, 14603-1051, US

(73) Assignee: University of Rochester, Rochester, NY (US)

(21) Appl. No.: 11/204,187

(22) Filed: Aug. 15, 2005

Related U.S. Application Data
(63) Continuation of application No. 09/606,129, filed on Jun. 28, 2000.
(60) Provisional application No. 60/141,309, filed on Jun. 28, 1999.
(60) Provisional application No. 60/163,223, filed on Nov. 3, 1999.

(51) Int. Cl. A61K 38/44; A61K 48/00

(52) U.S. Cl. 424/94.4; 514/44

(57) ABSTRACT

One aspect of the present invention relates to a method of regulating protein kinase C activity that includes contacting human protein kinase C selected from the group of isozymes α, β, and γ with a mammalian biliverdin reductase or a fragment thereof with protein kinase C regulatory activity.

This application is a continuation of U.S. patent application Ser. No. 09/606,129 filed Jun. 28, 2000, which claims the benefit of U.S. Provisional Patent Application Ser. Nos. 60/141,309, filed Jun. 28, 1999, and 60/163,223, filed Nov. 3, 1999, each of which is hereby incorporated by reference.

This work was supported by the U.S. National Institutes of Health Grant Nos. ES04066 and ES04391. The U.S. Government may have certain rights in the invention.

### FIELD OF THE INVENTION

The present invention relates to novel fragments and variants of biliverdin reductase, as well as novel methods of using biliverdin reductase, or fragments or variants thereof, in regulating protein kinase activity, regulating cell differentiation, growth or signaling, treating dysfunctional or diseased cells, and inhibiting cell death following a stroke/ischemic event.

### BACKGROUND OF THE INVENTION

Biliverdin reductase (“BVR”) catalyzes reduction of the γ-meso bridge of biliverdin, an open tetrapyrrole, to produce bilirubin (Singleton et al., J. Biol. Chem. 240: 47890-4789 (1965); Tenhunen et al., Biochemistry 9:298-303 (1970); Colleran et al., Biochem J. 119:16P-19P (1970); Kutty et al., J. Biol. Chem. 256:3956-3962 (1981); Buldain et al., Eur. J. Biochem. 156:179-184 (1986); Noguchi et al., Biochem J. 86:833-839 (1989)). In mammals, the oxidative cleavage of heme is catalyzed by the heme oxygenase system (Maines, Ann. Rev. Pharmacol. Toxicol. 37:517-554 (1997)). Because open tetrapyrroles are generally believed to be devoid of biological functions, the enzymes that catalyze their formation have not traditionally been in the main stream of research activity. In plants, however, biliverdin analogues, phytochromobilins, function in photoregulatory capacity (Terry et al., J. Biol. Chem. 266:22215-22221 (1991); Cornejo et al., J. Biol. Chem. 267:14790-14798 (1992)). Molecular cloning and biochemical analyses have shown that the enzyme, which in human is a 296 residue polypeptide, is highly conserved both at its primary structure and at its unique catalytic properties (Fakhrai et al., J. Biol. Chem. 267:4023-4029 (1992); McCoubrey et al., Eur. J Biochem. 222:597-603 (1994); McCoubrey et al., Gene 160:235-240 (1995); Maines et al., Eur. J. Biochem. 235:372-381 (1996)). BVR is the only enzyme described to date with dual pH/dual adenine nucleotide cofactor requirements (Kutty et al., J. Biol. Chem. 256:3956-3962 (1981); Fakhrai et al., J. Biol. Chem. 267:4023-4029 (1992); Maines et al., Eur. J. Biochem. 235:372-381 (1996); Huang et al., J. Biol. Chem. 264:7844-7849 (1989)). The reductase uses NADH in the acidic range (optimum range ˜pH 6.0-6.7), whereas NADPH is utilized in the basic range (optimum range ˜pH 8.5-8.7). BVR, which is a zinc metalloprotein (Maines et al., Eur. J. Biochem. 235:372-381 (1996)), possesses a His.Cys.Xaa₁₀.Cys.His or His.Cys.Xaa₁₀.Cys.Cys motif in the carboxy terminal third of the protein, which is similar to the zinc binding motif of protein kinase C (Hubbard et al., Science 254:1776-1779 (1991)) and may be the site of interaction of BVR with zinc.

BVR was previously thought to be simply a house-keeping enzyme found in most mammalian cells in excess of, or in disproportionate levels to, heme oxygenase isozymes (Ewing et al., J. Neurochem. 61:1015-1023 (1993)). Yet it has the above-noted noted unique and uncommon properties. Examination of the primary structure of human BVR, which recently became available (Maines et al., Eur. J. Biochem. 235:372-381 (1996)), revealed the presence of consensus sequences that are conserved in protein kinases, the most notable one being the Gly.Xaa.Gly¹⁷.Xaa.Xaa.Gly motif near the N terminus of the protein that is found invariably in all kinases (Kamps et al., Nature 310:589-592 (1984); Hunter et al., Ann. Rev. Biochem. 54:897-930 (1985); Schlessinger, Trend. Biochem. Sci. 13:443-447 (1988); Hanks et al., Science 241:42-52 (1988); Yarden et al., Annu. Rev. Biochem. 57:443-478 (1988); Hanks et al., Methods Enzymol. 200:38-62 (1991)). A valine residue is present in BVR just 2 positions downstream from the last glycine. A valine residue is invariant at the corresponding position, as in BVR, in the family of kinases that phosphorylate G-protein coupled receptors (Garcia-Bustos et al., Biochim. Biophys. ACTA 1071:83-101 (1991)). Database search results also identified additional similarities with PKGs, including a cluster of charged residues (Lys²²⁴.Arg.Asn.Arg) in the carboxy terminus of BVR. Such clusters are a characteristic of the nuclear localization signal (“NLS”) (Garcia-Bustos et al., Biochim. Biophys. ACTA 1071:83-101 (1991)).

The present invention is directed to identifying previously unrecognized BVR activities and properties, thereby determining novel therapeutic uses for BVR and otherwise overcoming deficiencies in the relevant art.

### SUMMARY OF THE INVENTION

One aspect of the present invention relates to a biliverdin reductase fragment or variant including a biliverdin reductase fragment possessing one or more activities of full length biliverdin reductase or a biliverdin reductase variant which includes one or more amino acid substitutions affecting one or more activities of full length biliverdin reductase. Expression systems and host cells containing a heterologous DNA molecule encoding the biliverdin reductase fragment or variant are disclosed. Isolated antibodies or binding portions thereof raised against the biliverdin reductase fragment or variant are also described.

A further aspect of the present invention relates to a method of regulating protein kinase activity which includes contacting a protein kinase with biliverdin reductase, or fragment or variant thereof, under conditions effective to regulate protein kinase activity.

Another aspect of the present invention relates to a method of regulating cell differentiation, growth, or signaling which includes contacting a cell with biliverdin reductase, or fragment or variant thereof, under conditions effective to regulate cell differentiation, growth, or signaling.

Yet another aspect of the present invention relates to a method of treating cellular dysfunction or disease which includes contacting a dysfunctional or diseased cell with biliverdin reductase, or fragment or variant thereof, under conditions effective to treat or immolate the dysfunctional or diseased cell.

Still another aspect of the present invention relates to a method of treating cells following stroke or an ischemic event which includes contacting a cell with biliverdin reductase, or fragment or variant thereof, under conditions effective to inhibit cell damage following stroke or an ischemic event.

With the identification of biliverdin reductase as an enzyme having activity not only in the heme oxygenase pathway, but also as a kinase surprisingly capable of autophosphorylation and a moderator of protein kinase activity, it is now apparent that biliverdin reductase plays a number of roles involved, among others, in cellular maintenance, signaling, cell differentiation, and cell proliferation. Due to its involvement with such diverse cellular processes, biliverdin reductase, as well as fragments or variants thereof, can be used to treat dysfunctional, diseased, or distressed cells for purposes of treating a number of diseases or disorders. The identification of different functional domains having various activities, as well as the identification of variants having variably affected (either enhanced or diminished) activities will allow for tailoring of treatments for dysfunctional, diseased, or distressed cells. Inhibitors of kinases and phosphorylation and signal transduction can be developed as anti-tumor candidates; whereas activators of signal transduction pathways can be developed for purposes of promoting cell growth and differentiation. Because heme oxygenase enzymes are phosphorylated and are upstream from signal transduction pathway enzymes that are regulated themselves by phosphorylation, kinase activity of BVR can be applied to a host of functions. This includes the various uses of heme oxygenase including, without limitation, prolonging transplanted organ half-life, treating jaundice and various other pathological disorders. Heme oxygenase activity has been implicated in a vast number of cellular functions ranging from inflammatory response, allograft rejection, carcinogenesis, neuroendocrine functions, and neuronal signaling.

### BRIEF DESCRIPTION OF THE DRAWINGS

FIGS. 1 is an image of an SDS-PAGE of hBVR subjected to immunoblotting. Phosphorylation molecular weight markers are shown on the left side (panel a) and hBVR immunoblotting on the right side (panel b). Immunoblotting used 2 μg of hBVR with a mixture (2 μg/ml each) of anti-phosphotyrosine, anti-phosphothreonine, and anti-phosphoserine (“anti-phospho mix”).

FIG. 2 is an image illustrating that rBVR is autophosphorylated. Purified rat liver BVR (40 μg) was subjected to gel electrophoresis and transferred to PVDF membrane, after which the membrane bound protein was denatured and renatured and incubated in the presence of [γ³²P]-ATP. The membrane was processed as described infra and exposed to X-Omat AR film (panel a). Panel b illustrates a Coomasie Blue stained SDS-PAGE of the BVR preparation.

FIGS. 3A-C are graphs depicting the effects of mutation of Gly¹⁷, Ser¹⁴⁹, and Lys²⁹⁶ on BVR catalytic activity. Purified preparations of recombinant wild type and mutant BVR with substitution of Gly¹⁷ (FIG. 3A), Ser¹⁴⁹ (FIG. 3B), or Lys²⁹⁶ (FIG. 3C) with Ala were assessed for BVR activity at pH 6.5 using NADH as cofactor and at pH 8.7 with NADPH as cofactor. Values presented are in percent wild type activity for each pH. The specific activity of the wild type preparation was 1744 nmol bilirubin produced min⁻¹ mg⁻¹ at pH 6.5 and 1147 nmol bilirubin produced min⁻¹ mg⁻¹ at pH 8.7. Values presented are representative of 2-4 determinations.

FIGS. 4A-F are images of wild type and transfected Hela cells following fluorescence immunocytochemistry, which illustrate that presence of the putative nuclear localization signal is necessary, but not sufficient for biliverdin reductase translocation into the nucleus. Constructs were made in pcDNA3, expressing hemagglutinin tagged: full length wild type human BVR or a full length protein in which mutations were introduced in the stretch of BVR amino acids encompassing either the putative NLS motif or the C-terminal 94 amino acids of BVR. After 48 h, the Hela cells were treated for 5 min with 8-bromo-cGMP and then harvested, followed by fluorescence immunocytochemistry as described infra. FIGS. 4A-B show cells expressing wild type BVR before treatment (4A) and after treatment (4B). FIGS. 4C-D show cells expressing NLS mutant BVR before treatment (4C) and after treatment (4D). FIGS. 4E-F show cells expressing the BVR carboxy terminal fragment before treatment (4E) and after treatment (4F).

FIGS. 5A-B are images of purified BVR wild type and mutant autophosphorylation. 20 μg aliquots of wild type BVR and Lys²⁹⁶ mutant BVR, and 10 μg of the Ser¹⁴⁹ mutant BVR were loaded onto membrane. FIG. 5A is a blot probed with anti-phospho mix as the primary antibody and FIG. 5B depicts the membrane counter-stained with manganese chloride and photographed with transmitted light.

FIG. 6 is a pair of images of purified BVR which illustrate that its microheterogeneity, as demonstrated by two-dimensional electrophoresis (bottom panel), is due to its phosphorylation (top panel) with the anti-phospho mix.

FIG. 7 is an image of an SDS-PAGE immunoblot which illustrates that biliverdin reductase is a serine-, threonine-, tyrosine-phosphoprotein. Purified rat liver BVR (1-3 μg/ml each) was subjected to SDS-PAGE and immunoblotting with the (anti-phospho mix (2 μg/ml each) (panel a), anti-phosphotyrosine (panel b), anti-phosphoserine (panel c), and anti-phosphothreonine (panel c).

FIG. 8 is an image illustrating that Gly¹⁷ is involved in phosphophorylation of BVR. Autokinase activity of the reductase as a function of Gly¹⁷ was measured using wild type hBVR or a Gly¹⁷→Ala mutant. Purified preparations of expressed wild type or Gly¹⁷ mutant BVR were subjected to SDS-PAGE immunoblotting using as the probe either antiphospho mix (panel a), anti-phosphotyrosine (panel b), anti-phosphoserine (panel c), or anti-phosphothreonine (panel d). Molecular markers are shown in panel e. 2 μg protein was loaded in each lane.

FIG. 9 is an image of an SDS-PAGE immunoblot which illustrates that heme oxygenase-1 and -2 are phosphoproteins. Purified rat liver BVR, HO-1, and HO-2 (4 μg each) were subjected to SDS-PAGE and Western analyses using anti-phospho mix as the primary antibody.

FIGS. 10A-B are images of SDS-PAGE and immunoblots which illustrate that BVR binds protein kinase C. Purified rat liver BVR was subjected to SDS-PAGE and was analyzed for protein kinase C binding using an overlay assay as detailed infra. Binding was examined by Western blotting using anti-PKC (FIG. 10A) or anti-BVR (FIG. 10B) as primary antibodies.

FIGS. 11A-C are graphs depicting the role of BVR in increasing protein kinase C activity. FIG. 11A depicts the dose-dependence of PKC activity in the presence of BVR or PKC inhibitor peptide (“PKCI”) in an assay system containing 0.5 μg/ml PKC, 50 μM unlabeled ATP, 5 μCi[³²γP]-ATP and 1 mg/ml MBP and carried out as described infra. FIGS. 11B-C are the kinetic analyses of PKC activity carried out with respect to MBP (FIG. 11B) and with respect to ATP (FIG. 11C) in the presence or absence of 50 μM BVR.

FIGS. 12A-C are graphs illustrating the effect of various rBVR fragments on PKC activity. Protein kinase C was incubated at 30° C. with buffer or with the indicated peptides (50 μM) for 15 min prior to addition to a kinase assay using MBP as substrate. FIG. 12A depicts the relative activity for each sample normalizing to that of the PKC and buffer control value. FIGS. 12B-C are the kinetic analyses of PKC with respect to substrate MBP (FIG. 12B) and ATP (FIG. 12C) in the presence or absence of BVR peptide fragments (50 μM). BVR1 peptide corresponds to SEQ. ID. No. 34 and BVR2 peptide corresponds to SEQ. ID. No. 19.

FIGS. 13A-D are images showing an immunohistochemical comparison of biliverdin reductase staining in normal kidney tissue and in renal carcinoma. 10 μm thick section of kidney were used for immunohistochemical analysis as described in infra. FIGS. 13A-B show tumor tissue at magnification of 4× (13A) and 100× (13B). FIGS. 13C shows tissue surrounding the tumor at magnification of 40×, with immunostaining of neutrophils (arrow) but not that of erythrocytes (arrowhead). FIG. 13D shows normal kidney tissue at magnification of 40×.

FIG. 14A-D are images (at 100× magnification) depicting the expression of biliverdin reductase in leukocytes. Tumor tissue was double immunostained for biliverdin reductase and CD antisera, and normal tissue was immunostained for the reductase as described infra. FIG. 14A displays anti-reductase and anti-CD68 double staining which identifies macrophage (arrow), neutrophils (arrowhead), and monocyte. FIG. 14B displays anti-reductase and anti-CD3 double staining, which identifies T cells staining for both (arrow) and neutrophils staining for reductase (arrowhead). FIG. 14C displays anti-reductase and anti-CD45, which identifies neutrophils staining for both (arrow) and lymphocytes staining with CD45 (arrowhead). FIG. 14D displays reductase staining in circulating leukocytes (arrow). Erythrocytes (arrowhead) do not stain.

FIGS. 15A-D are images and graphs illustrating the extent of biliverdin reductase expression or activity in tumor tissue. FIG. 15A shows a Northern blot analysis of mRNA. Poly(A)+RNA was isolated from pooled fractions of kidney tissue with the visible tumor and portions of tissue that did not have visible tumor. Lanes 1 and 2 contained poly(A)+RNA obtained from tissue surrounding the tumor and the tumor tissue, respectively. Signals were quantitated and normalized to that of actin. An increase of 175% in transcript level was documented for the tumor. Noteworthy is the increase in actin in the tumor tissue which suggest cellular transformation in cytoskeleton of malignant cells. FIG. 15B shows a Western blot analysis of protein. The cytosol fraction was prepared from the tumor and from a distant area of the same kidney without visible tumor. The preparations were subjected to SDS-gel electrophoresis, followed by electroblotting. Blot was developed using rabbit antibody to human kidney biliverdin reductase. Purified E. coli expressed human biliverdin reductase fusion protein (Maines et al., Eur. J. Biochem. 235:372 (1996), which is hereby incorporated by reference) was used as the standard. Lanes: 1 and 2=purified E. coli expressed fusion human biliverdin reductase; 3=molecular weight markers; 4=tissue surrounding tumor; 5=tumor tissue. FIG. 15C-D shows pH-dependent activity of biliverdin reductase at pH 6.7 and 8.7, measured as described infra using NADH at pH 6.7 (15C) and NADPH at pH 8.7 (15D). The cytosol fraction obtained as above was used as the enzyme source. Data represent the mean of 2 separate samples.

FIGS. 16A-C are a graph and images illustrating stroke volume and areas of anterior-posterior distribution of ischemic damage in mice 6, 12 and 24 h after MCAo. FIG. 16A graphically illustrates stroke volume at 6, 12 and 24 h after MCAo, particularly the delayed maturation of the ischemic lesions over the course of 24 h. Asterisks indicate statistical significance at the level of p<0.05. FIG. 16B is an image showing BVR immunostaining in control tissue. FIG. 16C is an image showing BVR immunostaining 24 h after MCAo, particularly the increased immunoreactivity for BVR within areas bordering the ischemic lesion in the cortex (arrowhead) and caudate nucleus (arrows).

FIGS. 17A-D are images which illustrate the persistent increase in immunostaining for BVR in ischemic caudate. Specimens of mouse brains at 0 (FIG. 17A, objective 20×), 6 (FIG. 17B, objective 20×), 12 (FIG. 17C, objective 40×) and 24 h (FIG. 17D, objective 40×) after MCAo were immunostained for BVR as described infra. An increase in number and intensity of BVR immunostaining in the ischemic caudate at all time points after MCAo is evident when compared with 0 time control. Arrow=BVR (+) neuron; arrowhead=microvessels; p=ischemic penumbra; and c=ischemic core.

FIGS. 18A-B are an image and a graph which illustrate increased expression of BVR and its correlation with neuronal cell survival in cortical layers 3 and 5. FIG. 18A is an image (objective 20×) showing BVR immunoreactivity in ischemic hemisphere cortical neurons in layers 3 (top layer; marked crtx 3) and 5 (bottom layer; marked crtx 5) 6 h after MCAo. Cell bodies and long cellular processes (marked by arrows) emanate from layer 5 neurons towards pyramidal neurons of layer 3. FIG. 18B is a graph illustrating the time dependent increase in proportion of neurons double-labeled for thionin and BVR in cortical layers 3 and 5. Asterisks indicate statistical significance at the level of p<0.05.

FIGS. 19A-F are images which illustrate the increase in BVR immunoreactivity in neurons in substantia nigra, in Purkinje neurons of the cerebellum and in neurons in CIC nucleus after MCAo. Mouse brain BVR immunostaining was carried out using control tissue (FIGS. 19A-C, objective 40×) and tissue 6 h after induction of MCAo (FIGS. 19D-F, objective 40×). When compared with normal tissue, in ischemic tissue there is an increase in intensity of BVR staining of neurons of substantia nigra (FIG. 19A versus 19D), Purkinje neurons (FIG. 19B versus 19E), and in association with nucleus of neurons of the CIC region (FIG. 19C versus 19F). Arrow=Purkinje neuron.

FIGS. 20A-F are images which illustrate the spatial and temporal distribution of BVR immunoreactivity and histochemical staining for iron and lipid peroxidation in the ischemic cortex. Brain tissue from mice subjected to MCAo was analyzed after 6 or 24 h for BVR immunoreactivity (FIGS. 20A-B, objective 40×), iron staining (FIGS. 20C-D, objective 10×), and Schiff's reagent staining for detection of lipid peroxidation activity (FIGS. 20E-F, objective 20×). FIGS. 20A, 20C, and 20E are 6 h post MCAo and FIGS. 20B, 20D, and 20E are 24 h after MCAo. Staining for lipid peroxidation was primarily observed at the rim of the ischemic penumbra at 24 h post MCAo (FIG. 20E versus 20F). Area marked between 2 arrows in FIG. 20F corresponds to the rim of ischemic penumbra in FIG. 20B. Insert in FIG. 20C corresponds to higher magnification of cortical layer 3 neurons (objective 20×). Abbreviations: c=ischemic core; p=ischemic penumbra; crtx3=cortical layer 3; crtx5=cortical layer 5; arrowhead=BVR positive neuron at the border zone of ischemic core and penumbra.

FIGS. 21A-E are images which illustrate BVR transcript, protein, and activity levels in ischemic brain. FIG. 21A is a Northern blot analysis carried out using poly(A+) isolated from contralateral and ipsilateral hemispheres of mice at 6 and 24 h after MCAo. Ten microgram of poly(A+) RNA was loaded into each lane. Blot was hybridized with ³²P-labeled rat BVR cDNA probe (Fakhrai et al., J. Biol. Chem. 267:4023-4029 (1992), which is hereby incorporated by reference), and then probed with ≢0 c-actin, which was used as the loading control. For each contralateral hemisphere sample, the ratio of BVR message (at 1.5 kb) relative to that of actin mRNA (at 2.1 kb) was quantitated and was assigned a value of 1. Such ratio for the corresponding ipsilateral was compared to this value. Lanes: 1=contralateral hemisphere; 2=ischemic hemisphere. FIGS. 21B-C show BVR mRNA in situ hybridization performed on 8 μm thick paraffin embedded specimens 6 h (FIG. 21B, objective 20×) and 24 h (FIG. 21C, objective 20×) post MCAo using digoxigenin-labeled single-stranded sense and antisense probes. Only background staining was detected when sense oligonucleotide was used as hybridization probe. FIG. 21D is a Western blot analysis of BVR performed using anti-rat BVR antibody (Huang et al., J. Biol. Chem. 264:7844-7849 (1989), which is hereby incorporated by reference) and 105,000× supernatant fraction obtained from the ipsolateral and contralateral hemispheres of brain at 6, 12 or 24 h after MCAo. The absence of a time-dependent change in BVR antibody immunoreactive band at 32 kDa after MCAo (lanes 2, 4 and 6) is readily identified when compared to samples obtained from contralateral hemisphere (lanes 1, 3 and 5). Lanes: 1 and 2=6 h; 3 and 4=12 h; 5 and 6=24 h after MCAo. Purified rat BVR was used as standard (St). Mr=molecular weight (Rainbow) markers. FIG. 21E is a measurement of NADH-dependent activity of BVR in the ischemic brain hemisphere and the contralateral hemisphere at 6, 12 and 24 h after induction of MCAo was carried out using brain 105,000× supernatant fractions. The unit of measurement is nmol bilirubin formed per min per mg protein.

FIGS. 22A-F are images illustrating control Hela cells and transfected Hela cells expressing antisense BVR RNA at magnification 1 Ox (FIGS. 22A-B), 40× (FIGS. 22C-D), and 100× (FIGS. 22E-F). Control cells appear morphologically normal (FIGS. 22A, 22C, and 22E), whereas antisense BVR RNA cells appear stressed (FIGS. 22B, 22D, and 22F).

FIGS. 23A-H are images illustrating the response of the control Hela cells and antisense BVR RNA Hela cells in response to hematin (FIGS. 23A-D), sodium arsenite (FIGS. 23E-F), and menadione (FIGS. 23G-H).

### DETAILED DESCRIPTION OF THE INVENTION

The present invention relates to the identification of previously unrecognized activities and properties of biliverdin reductase, including various therapeutic uses for BVR, as well as variant forms and fragments of BVR which possess differential patterns of activity.

One form of human biliverdin reductase (“hBVR”) has an amino acid sequence corresponding to SEQ. ID. No. 1 as follows:

| Met | Asn | Ala | Glu | Pro | Glu | Arg | Lys | Phe | Gly | Val | Val | Val | Val | Gly | Val |
|---|---|---|---|---|---|---|---|---|---|---|---|---|---|---|---|
| 1 | | | | 5 | | | | | 10 | | | | | 15 | |
| Gly | Arg | Ala | Gly | Ser | Val | Arg | Met | Arg | Asp | Leu | Arg | Asn | Pro | His | Pro |
| | | | 20 | | | | | 25 | | | | 30 | | | |
| Ser | Ser | Ala | Phe | Leu | Asn | Leu | Ile | Gly | Phe | Val | Ser | Arg | Arg | Glu | Leu |
| | | 35 | | | | | 40 | | | | | 45 | | | |
| Gly | Ser | Ile | Asp | Gly | Val | Gln | Gln | Ile | Ser | Leu | Glu | Asp | Ala | Leu | Ser |
| | 50 | | | | | 55 | | | | | 60 | | | | |
| Ser | Gln | Glu | Val | Glu | Val | Ala | Tyr | Ile | Cys | Ser | Glu | Ser | Ser | Ser | His |
| 65 | | | | | 70 | | | | | 75 | | | | | 80 |
| Glu | Asp | Tyr | Ile | Arg | Gln | Phe | Leu | Asn | Ala | Gly | Lys | His | Val | Leu | Val |
| | | | | 85 | | | | | 90 | | | | | 95 | |
| Glu | Tyr | Pro | Met | Thr | Leu | Ser | Leu | Ala | Ala | Ala | Gln | Glu | Leu | Trp | Glu |
| | | | 100 | | | | | 105 | | | | | 110 | | |
| Leu | Ala | Glu | Gln | Lys | Gly | Lys | Val | Leu | His | Glu | Glu | His | Val | Glu | Leu |
| | | 115 | | | | | 120 | | | | | 125 | | | |
| Leu | Met | Glu | Glu | Phe | Ala | Phe | Leu | Lys | Lys | Glu | Val | Val | Gly | Lys | Asp |
| | 130 | | | | | 135 | | | | | 140 | | | | |
| Leu | Leu | Lys | Gly | Ser | Leu | Leu | Phe | Thr | Ser | Asp | Pro | Leu | Glu | Glu | Asp |
| 145 | | | | | 150 | | | | | 155 | | | | | 160 |
| Arg | Phe | Gly | Phe | Pro | Ala | Phe | Ser | Gly | Ile | Ser | Arg | Leu | Thr | Trp | Leu |
| | | | | 165 | | | | | 170 | | | | | 175 | |
| Val | Ser | Leu | Phe | Gly | Glu | Leu | Ser | Leu | Val | Ser | Ala | Thr | Leu | Glu | Glu |
| | | | 180 | | | | | 185 | | | | | 190 | | |
| Arg | Lys | Glu | Asp | Gln | Tyr | Met | Lys | Met | Thr | Val | Cys | Leu | Glu | Thr | Glu |
| | | 195 | | | | | 200 | | | | | 205 | | | |
| Lys | Lys | Ser | Pro | Leu | Ser | Trp | Ile | Glu | Glu Lys | Gly | Pro | Gly | Leu | Lys | |
| | 210 | | | | | 215 | | | | | 220 | | | | |
| Arg | Asn | Arg | Tyr | Leu | Ser | Phe | His | Phe | Lys | Ser | Gly | Ser | Leu | Glu | Asn |
| 225 | | | | | 230 | | | | | 235 | | | | | 240 |
| Val | Pro | Asn | Val | Gly | Val | Asn | Lys | Asn | Ile | Phe | Leu | Lys | Asp | Gln | Asn |
| | | | | 245 | | | | | 250 | | | | | 255 | |
| Ile | Phe | Val | Gln | Lys | Leu | Leu | Gly | Gln | Phe | Ser | Glu | Lys | Glu | Leu | Ala |
| | | | 260 | | | | | 265 | | | | | 270 | | |
| Ala | Glu | Lys | Lys | Arg | Ile | Leu | His | Cys | Leu | Gly | Leu | Ala | Glu | Glu | Ile |
| | | 275 | | | | | 280 | | | | | 285 | | | |
| Gln | Lys | Tyr | Cys | Cys | Ser | Arg | Lys | | | | | | | | |
| | 290 | | | | | 295 | | | | | | | | | |

Heterologous expression and isolation of hBVR is described in Maines et al., Eur. J. Biochem. 235(1-2):372-381 (1996); Maines et al., Arch. Biochem. Biophys. 300(1):320-326 (1993), which are hereby incorporated by reference. The DNA molecule encoding this form of hBVR has a nucleotide sequence corresponding to SEQ. ID. No. 2 as follows:

| | | | | | | |
|---|---|---|---|---|---|---|
| ggggtggcgc | ccggagctgc | acggagagcg | tgcccgtcag | tgaccgaaga | agagaccaag | 60 |
| atgaatgcag | agcccgagag | gaagtttggc | gtggtggtgg | ttggtgttgg | ccgagccggc | 120 |
| tccgtgcgga | tgagggactt | gcggaatcca | cacccttcct | cagcgttcct | gaacctgatt | 180 |
| ggcttcgtgt | cgagaaggga | gctcgggagc | attgatggag | tccagcagat | ttctttggag | 240 |
| gatgctcttt | ccagccaaga | ggtggaggtc | gcctatatct | gcagtgagag | ctccagccat | 300 |
| gaggactaca | tcaggcagtt | ccttaatgct | ggcaagcacg | tccttgtgga | ataccccatg | 360 |
| acactgtcat | tggcggccgc | tcaggaactg | tgggagctgg | ctgagcagaa | aggaaaagtc | 420 |
| ttgcacgagg | agcatgttga | actcttgatg | gaggaattcg | ctttcctgaa | aaaagaagtg | 480 |
| gtggggaaag | acctgctgaa | agggtcgctc | ctcttcacat | ctgacccgtt | ggaagaagac | 540 |
| cggtttggct | tccctgcatt | cagcggcatc | tctcgactga | cctggctggt | ctccctcttt | 600 |
| ggggagcttt | ctcttgtgtc | tgccactttg | gaagagcgaa | aggaagatca | gtatatgaaa | 660 |
| atgacagtgt | gtctggagac | agagaagaaa | agtccactgt | catggattga | agaaaaagga | 720 |
| cctggtctaa | aacgaaacag | atatttaagc | ttccatttca | agtctgggtc | cttggagaat | 780 |
| gtgccaaatg | taggagtgaa | taagaacata | tttctgaaag | atcaaaatat | atttgtccag | 840 |
| aaactcttgg | gccagttctc | tgagaaggaa | ctggctgctg | aaaagaaacg | catcctgcac | 900 |
| tgcctggggc | ttgcagaaga | aatccagaaa | tattgctgtt | caaggaagta | agaggaggag | 960 |
| gtgatgtagc | acttccaaga | tggcaccagc | atttggttct | tctcaagagt | tgaccattat | 1020 |
| ctctattctt | aaaattaaac | atgttgggga | aacaaaaaaa | aaaaaaaaaa | | 1070 |

The open reading frame which encodes hBVR of SEQ. ID. No. 1 extends from nt 1 to nt 888.

Another form of hBVR has an amino acid sequence according to SEQ. ID. No. 3 as follows:

| Met | Asn | Thr | Glu | Pro | Glu | Arg | Lys | Phe | Gly | Val | Val | Val | Val | Gly | Val |
|---|---|---|---|---|---|---|---|---|---|---|---|---|---|---|---|
| 1 | | | | 5 | | | | | 10 | | | | | 15 | |
| Gly | Arg | Ala | Gly | Ser | Val | Arg | Met | Arg | Asp | Leu | Arg | Asn | Pro | His | Pro |
| | | | 20 | | | | | 25 | | | | | 30 | | |
| Ser | Ser | Ala | Phe | Leu | Asn | Leu | Ile | Gly | Phe | Val | Ser | Arg | Arg | Glu | Leu |
| | | 35 | | | | | 40 | | | | | 45 | | | |
| Gly | Ser | Ile | Asp | Gly | Val | Gln | Gln | Ile | Ser | Leu | Glu | Asp | Ala | Leu | Ser |
| | 50 | | | | | 55 | | | | | 60 | | | | |
| Ser | Gln | Glu | Val | Glu | Val | Ala | Tyr | Ile | Cys | Ser | Glu | Ser | Ser | Ser | His |
| 65 | | | | | 70 | | | | | 75 | | | | | 80 |
| Glu | Asp | Tyr | Ile | Arg | Gln | Phe | Leu | Asn | Ala | Gly | Lys | His | Val | Leu | Val |
| | | | | 85 | | | | | 90 | | | | | 95 | |
| Glu | Tyr | Pro | Met | Thr | Leu | Ser | Leu | Ala | Ala | Ala | Gln | Glu | Leu | Trp | Glu |
| | | | 100 | | | | | 105 | | | | | 110 | | |
| Leu | Ala | Glu | Gln | Lys | Gly | Lys | Val | Leu | His | Glu | Glu | His | Val | Glu | Leu |
| | | 115 | | | | | 120 | | | | | 125 | | | |
| Leu | Met | Glu | Glu | Phe | Ala | Phe | Leu | Lys | Lys | Glu | Val | Val | Gly | Lys | Asp |
| | 130 | | | | | 135 | | | | | 140 | | | | |
| Leu | Leu | Lys | Gly | Ser | Leu | Leu | Phe | Thr | Ala | Gly | Pro | Leu | Glu | Glu | Glu |
| 145 | | | | | 150 | | | | | 155 | | | | | 160 |
| Arg | Phe | Gly | Phe | Pro | Ala | Phe | Ser | Gly | Ile | Ser | Arg | Leu | Thr | Trp | Leu |
| | | | | 165 | | | | | 170 | | | | | 175 | |
| Val | Ser | Leu | Phe | Gly | Glu | Leu | Ser | Leu | Val | Ser | Ala | Thr | Leu | Glu | Glu |
| | | | 180 | | | | | 185 | | | | | 190 | | |
| Arg | Lys | Glu | Asp | Gln | Tyr | Met | Lys | Met | Thr | Val | Cys | Leu | Glu | Thr | Glu |
| | | 195 | | | | | 200 | | | | | 205 | | | |
| Lys | Lys | Ser | Pro | Leu | Ser | Trp | Ile | Glu | Glu | Lys | Gly | Pro | Gly | Leu | Lys |
| | 210 | | | | | 215 | | | | | 220 | | | | |
| Arg | Asn | Arg | Tyr | Leu | Ser | Phe | His | Phe | Lys | Ser | Gly | Ser | Leu | Glu | Asn |
| 225 | | | | | 230 | | | | | 235 | | | | | 240 |
| Val | Pro | Asn | Val | Gly | Val | Asn | Lys | Asn | Ile | Phe | Leu | Lys | Asp | Gln | Asn |
| | | | | 245 | | | | | 250 | | | | | 255 | |
| Ile | Phe | Val | Gln | Lys | Leu | Leu | Gly | Gln | Phe | Ser | Glu | Lys | Glu | Leu | Ala |
| | | | 260 | | | | | 265 | | | | | 270 | | |
| Ala | Glu | Lys | Lys | Arg | Ile | Leu | His | Cys | Leu | Gly | Leu | Ala | Glu | Glu | Ile |
| | | 275 | | | | | 280 | | | | | 285 | | | |
| Gln | Lys | Tyr | Cys | Cys | Ser | Arg | Lys | | | | | | | | |
| | 290 | | | | | 295 | | | | | | | | | |

This hBVR sequence is reported at Komuro et al., NCBI Accession No. GO2066, direct submission to the EMBL Data Library (1998), which is hereby incorporated by reference. Differences between the hBVR of SEQ. ID. No. 1 and the hBVR of SEQ. ID. No. 3 are at aa residues 3, 154, 155, and 160. Thus, residue 3 can be either alanine or threonine, residue 154 can be either alanine or serine, residue 155 can be either aspartic acid or glycine, and residue 160 can be either aspartic acid or glutamic acid.

One form of rat biliverdin reductase (“rBVR”) has an amino acid sequence corresponding to SEQ. ID. No. 4 as follows:

| Met | Asp | Ala | Glu | Pro | Lys | Arg | Lys | Phe | Gly | Val | Val | Val | Val | Gly | Val |
|---|---|---|---|---|---|---|---|---|---|---|---|---|---|---|---|
| 1 | | | | 5 | | | | | 10 | | | | | 15 | |
| Gly | Arg | Ala | Gly | Ser | Val | Arg | Leu | Arg | Asp | Leu | Lys | Asp | Pro | Arg | Ser |
| | | | 20 | | | | | 25 | | | | | 30 | | |
| Ala | Ala | Phe | Leu | Asn | Leu | Ile | Gly | Phe | Val | Ser | Arg | Arg | Glu | Leu | Gly |
| | | 35 | | | | | 40 | | | | | 45 | | | |
| Ser | Leu | Asp | Glu | Val | Arg | Gln | Ile | Ser | Leu | Glu | Asp | Ala | Leu | Arg | Ser |
| | 50 | | | | | 55 | | | | | 60 | | | | |
| Gln | Glu | Ile | Asp | Val | Ala | Tyr | Ile | Cys | Ser | Glu | Ser | Ser | Ser | His | Glu |
| 65 | | | | | 70 | | | | | 75 | | | | | 80 |
| Asp | Tyr | Ile | Arg | Gln | Phe | Leu | Gln | Ala | Gly | Lys | His | Val | Leu | Val | Glu |
| | | | | 85 | | | | | 90 | | | | | 95 | |
| Tyr | Pro | Met | Thr | Leu | Ser | Phe | Ala | Ala | Ala | Gln | Glu | Leu | Trp | Glu | Leu |
| | | | 100 | | | | | 105 | | | | | 110 | | |
| Ala | Ala | Gln | Lys | Gly | Arg | Val | Leu | His | Glu | Glu | His | Val | Glu | Leu | Leu |
| | | 115 | | | | | 120 | | | | | 125 | | | |
| Met | Glu | Glu | Phe | Glu | Phe | Leu | Arg | Arg | Glu | Val | Leu | Gly | Lys | Glu | Leu |
| | 130 | | | | | 135 | | | | | 140 | | | | |
| Leu | Lys | Gly | Ser | Leu | Arg | Phe | Thr | Ala | Ser | Pro | Leu | Glu | Glu | Glu | Arg |
| 145 | | | | | 150 | | | | | 155 | | | | | 160 |
| Phe | Gly | Phe | Pro | Ala | Phe | Ser | Gly | Ile | Ser | Arg | Leu | Thr | Trp | Leu | Val |
| | | | | 165 | | | | | 170 | | | | | 175 | |
| Ser | Leu | Phe | Gly | Glu | Leu | Ser | Leu | Ile | Ser | Ala | Thr | Leu | Glu | Glu | Arg |
| | | | 180 | | | | | 185 | | | | | 190 | | |
| Lys | Glu | Asp | Gln | Tyr | Met | Lys | Met | Thr | Val | Gln | Leu | Glu | Thr | Gln | Asn |
| | | 195 | | | | | 200 | | | | | 205 | | | |
| Lys | Gly | Leu | Leu | Ser | Trp | Ile | Glu | Glu | Lys | Gly | Pro | Gly | Leu | Lys | Arg |
| | 210 | | | | | 215 | | | | | 220 | | | | |
| Asn | Arg | Tyr | Val | Asn | Phe | Gln | Phe | Thr | Ser | Gly | Ser | Leu | GluGlu | Val | |
| 225 | | | | | 230 | | | | | 235 | | | | | 240 |
| Pro | Ser | Val | Gly | Val | Asn | Lys | Asn | Ile | Phe | Leu | Lys | Asp | Gln | Asp | Ile |
| | | | | 245 | | | | | 250 | | | | | 255 | |
| Phe | Val | Gln | Lys | Leu | Leu | Asp | Gln | Val | Ser | Ala | Glu | Asp | Leu | Ala | Ala |
| | | | 260 | | | | | 265 | | | | | 270 | | |
| Glu | Lys | Lys | Arg | Ile | Met | His | Cys | Leu | Gly | Leu | Ala | Ser | Asp | Ile | Gln |
| | | 275 | | | | | 280 | | | | | 285 | | | |
| Lys | Leu | Cys | His | Gln | Lys | Lys | | | | | | | | | |
| | 290 | | | | | 295 | | | | | | | | | |

Heterologous expression and isolation of rBVR is described in Fakhrai et al., J. Biol. Chem. 267(6):4023-4029 (1992), which is hereby incorporated by reference. The rBVR of SEQ. ID. No. 4 shares about 82% aa identity to the hBVR of SEQ. ID. No. 1, with variations in aa residues being highly conserved. The DNA molecule encoding this form of rBVR has a nucleotide sequence corresponding to SEQ. ID. No. 5 as follows:

| | | | | | | |
|---|---|---|---|---|---|---|
| ggtcaacagc | taagtgaagc | catatccata | gagagtttgt | gccagtgccc | caagatcctg | 60 |
| aacctctgtc | tgtcttcgga | cactgactga | agagaccgag | atggatgccg | agccaaagag | 120 |
| gaaatttgga | gtggtagtgg | ttggtgttgg | cagagctggc | tcggtgaggc | tgagggactt | 180 |
| gaaggatcca | cgctctgcag | cattcctgaa | cctgattgga | tttgtgtcca | gacgagagct | 240 |
| tgggagcctt | gatgaagtac | ggcagatttc | tttggaagat | gctctccgaa | gccaagagat | 300 |
| tgatgtcgcc | tatatttgca | gtgagagttc | cagccatgaa | gactatatac | ggcagtttct | 360 |
| gcaggctggc | aagcatgtcc | tcgtggaata | ccccatgaca | ctgtcatttg | cggcggccca | 420 |
| ggagctgtgg | gagctggccg | cacagaaagg | gagagtcctg | catgaggagc | acgtggaact | 480 |
| cttgatggag | gaattcgaat | tcctgagaag | agaagtgttg | gggaaagagc | tactgaaagg | 540 |
| gtctcttcgc | ttcacagcta | gcccactgga | agaagagaga | tttggcttcc | ctgcgttcag | 600 |
| cggcatttct | cgcctgacct | ggctggtctc | cctcttcggg | gagctttctc | ttatttctgc | 660 |
| caccttggaa | gagcgaaaag | aggatcagta | tatgaaaatg | accgtgcagc | tggagaccca | 720 |
| gaacaagggt | ctgctgtcat | ggattgaaga | gaaagggcct | ggcttaaaaa | gaaacagata | 780 |
| tgtaaacttc | cagttcactt | ctgggtccct | ggaggaagtg | ccaagtgtag | gggtcaataa | 840 |
| gaacattttc | ctgaaagatc | aggatatatt | tgttcagaag | ctcttagacc | aggtctctgc | 900 |
| agaggacctg | gctgctgaga | agaagcgcat | catgcattgc | ctggggctgg | ccagcgacat | 960 |
| ccagaagctt | tgccaccaga | agaagtgaag | aggaagcttc | agagacttct | gaagggggcc | 1020 |
| agggtttggt | cctatcaacc | attcaccttt | agctcttaca | attaaacatg | tcagataaac | 1080 |
| a | | | | | | 1081 |

The open reading frame which encodes rBVR of SEQ. ID. No. 4 extends from nt 1 to nt 885.

By way of example, hBVR of SEQ. ID. No. 1 is characterized by a number of functional domains, including putative and/or demonstrated phosphorylation sites from aa 15 to 20, aa 21 to 23, aa 44 to 46 or 47, aa 49 to 54, aa 58 to 61, aa 64 to 67, aa 78 to 81, aa 79 to 82, aa 189 to 192, aa 207 to 209, aa 214 to 217, aa 222 to 227, aa 236 to 241, aa 245 to 250, aa 267 to 269 or 270, and aa 294 to 296; a basic N-terminal domain characterized by aa 6 to 8; a hydrophobic domain characterized by aa 9 to 14 (FXVVVV, SEQ. ID. No. 6); a nucleotide binding domain characterized by aa 15 to 20. (GXGXXG, SEQ. ID. No. 7); an oxidoreductase domain characterized by aa 90 to 97 (AGKHVLVE, SEQ. ID. No. 8); a leucine zipper spanning aa 129 to 157 (LX₆LX₆KX₆LX₆L, SEQ. ID. No. 9); several kinase motifs, including aa 44 to 46 (SRR, SEQ. ID. No. 10), aa 147 to 149 (KGS, SEQ. ID. No. 11) and aa 162 to 164 (FGX, SEQ. ID. No. 12); a nuclear localization signal spanning aa 222 to 228 (GLKRNRY, SEQ. ID. No. 13); a myristylation site spanning aa 221 to 225 (PGLKR, SEQ. ID. No. 14); a zinc finger domain spanning aa 280 to 293 (HCX₁₀CC, SEQ. ID. No. 15); and substrate binding domains including, without limitation, a protein kinase C (“PKC”) enhancing domain spanning aa 275 to 281 (KKRIXHC, SEQ. ID. No. 16) and a PKC inhibiting domain spanning aa 289 to 296 (QKXCXXXK, SEQ. ID. No. 17). By way of sequence comparison and, in consideration of conserved substitutions, hBVR of SEQ. ID. No.3 and rBVR of SEQ. ID. No. 4 include similar functional domains. For example rBVR includes an identical hydrophobic domain, an identical nucleotide binding domain, an identical oxidoreductase domain, a conserved leucine zipper domain (with residue variations between L and K residues), identical or conserved kinase motifs, an identical nuclear localization signal, an identical myristylation site, a conserved zinc finger domain (with terminal C residue replaced by H), a conserved PKC enhancing domain, and a conserved PKC inhibiting domain.

DNA molecules encoding a BVR protein or polypeptide can also include a DNA molecule that hybridizes under stringent conditions to the DNA molecule having a nucleotide sequence of SEQ. ID. No. 2 or SEQ. ID. No. 5. An example of suitable stringency conditions is when hybridization is carried out at a temperature of about 37° C. using a hybridization medium that includes 0.9M sodium citrate (“SSC”) buffer, followed by washing with 0.2× SSC buffer at 37° C. Higher stringency can readily be attained by increasing the temperature for either hybridization or washing conditions or increasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency. Exemplary high stringency conditions include carrying out hybridization at a temperature of about 42° C. to about 65° C. for up to about 20 hours in a hybridization medium containing 1 M NaCl, 50 mM Tris-HCl, pH 7.4, 10 mM EDTA, 0.1% sodium dodecyl sulfate (SDS), 0.2% ficoll, 0.2% polyvinylpyrrolidone, 0.2% bovine serum albumin, and 50 μg/ml E. coli DNA, followed by washing carried out at between about 42° C. to about 65° C. in a 0.2×SSC buffer.

The BVR protein or polypeptide can also be a fragment of the above biliverdin reductase proteins or polypeptides or a variant thereof.

Fragments of BVR preferably contain one or more of the above-listed functional domains, and possess one or more of the activities of full length BVR. Suitable fragments can be produced by several means. Subclones of a gene encoding a known BVR can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), which are hereby incorporated by reference. The subclones then are expressed in vitro or in vivo in bacterial cells to yield a smaller protein or polypeptide that can be tested for a particular activity, e.g., converting biliverdin to bilirubin, modifying protein kinase C activity, etc., as discussed infra. See also Huang et al., J. Biol. Chem. 264:7844-7849 (1989), which is hereby incorporated by reference.

In another approach, based on knowledge of the primary structure of the protein, fragments of a BVR gene may be synthesized using the PCR technique together with specific sets of primers chosen to represent particular portions of the protein. Erlich et al., Science 252:1643-51 (1991), which is hereby incorporated by reference. These can then be cloned into an appropriate vector for expression of a truncated protein or polypeptide from bacterial cells as described above. For example, oligomers of at least about 15 to 20 nt in length can be selected from the nucleic acid molecules of SEQ. ID. No. 2 and SEQ ID. No. 5 for use as primers.

In addition, chemical synthesis can also be employed using techniques well known in the chemistry of proteins such as solid phase synthesis (Merrifield, J. Am. Chem. Assoc. 85:2149-2154 (1964), which is hereby incorporated by reference) or synthesis in homogenous solution (Houbenweyl, Methods of Organic Chemistry, ed. E. Wansch, Vol. 15, I and II, Thieme, Stuttgart (1987), which is hereby incorporated by reference).

Exemplary fragments include N-terminal, internal, and C-terminal fragments which possess one or more active domains of the whole BVR enzyme. One internal peptide fragment of rBVR includes the amino acid sequence KKRIMHC (SEQ. ID. No. 18), which corresponds to aa residues 274-280 of SEQ. ID. No. 4. An internal peptide fragment of rBVR includes the amino acid sequence KKRIMHC (SEQ. ID. No. 34), which corresponds to aa residues 275 to 281 of SEQ. ID. Nos. 1 and 3. These fragments possess activity as an enhancer of protein kinase C. One C-terminal peptide fragment of rBVR includes the amino acid sequence QKLCHQKK (SEQ. ID. No. 19), which corresponds to aa residues 288-295 of SEQ. ID. No. 4. A corresponding C-terminal fragment of hBVR includes the amino acid sequence QKYCCSRK (SEQ. ID. No.35), which corresponds to aa residues 289-296 of SEQ. ID. No. 1. This fragment possesses activity as an inhibitor of protein kinase C.

Variants of suitable BVR proteins or polypeptides can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of BVR. Variants of BVR can also be fragments of BVR which include one or more deletion, addition, or alteration of amino acids of the type described above. The BVR variant preferably contain a deletion, addition, or alteration of amino acids within one of the above-listed functional domains. The substituted or additional amino acids can be either L-amino acids, D-amino acids, or modified amino acids, preferably L-amino acids. Whether a substitution, addition, or deletion results in modification of BVR variant activity may depend, at least in part, on whether the altered amino acid is conserved. Conserved amino acids can be grouped either by molecular weight or charge and/or polarity of R groups, acidity, basicity, and presence of phenyl groups, as is known in the art.

Exemplary variants include the protein or polypeptides of SEQ. ID. Nos. 1, 3, and 4 which have single or multiple amino acid residue substitutions, including, without limitation, SEQ. ID. No. 1 as modified by one or more of the following variations: (i) Gly¹⁷→Ala within the nucleotide binding domain, (ii) Ser⁴⁴→Ala within one of the kinase motifs, (iii) Ser¹⁴⁹→Ala within the kinase motif of the leucine zipper, (iv) Cys⁷⁴→Ala within a substrate binding domain, (v) Lys⁹²His⁹³→Ala-Ala within the oxidoreductase motif, (vi) G²²²LKRNR²²⁷→VIGSTG within the nuclear localization signal, and (vii) Cys²⁸¹→Ala within the zinc finger domain, and Lys²⁹⁶→Ala at the C terminus within a substrate binding domain (i.e., protein kinase inhibitory domain).

Variants may also include, for example, a polypeptide conjugated to a signal (or leader) sequence at the N-terminal end of the protein which co-translationally or post-translationally directs transfer of the protein. The polypeptide may also be conjugated to a linker or other sequence for ease of synthesis, purification, identification, or therapeutic use (i.e., delivery) of the polypeptide.

The BVR protein or polypeptide can be recombinantly produced, isolated, and then purified, if necessary. When recombinantly produced, the biliverdin reductase protein or polypeptide is expressed in a recombinant host cell, typically, although not exclusively, a prokaryote.

When a prokaryotic host cell is selected for subsequent transformation, the promoter region used to construct the recombinant DNA molecule (i.e., transgene) should be appropriate for the particular host. The DNA sequences of eukaryotic promoters, as described infra for expression in eukaryotic host cells, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (“ATG”) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Mammalian cells can also be used to recombinantly produce BVR or fragments or variants thereof.

Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells. Suitable expression vectors for directing expression in mammalian cells generally include a promoter, as well as other transcription and translation control sequences known in the art. Common promoters include SV40, MMTV, metallothionein-1, adenovirus Ela, CMV, immediate early, immunoglobulin heavy chain promoter and enhancer, and RSV-LTR.

Regardless of the selection of host cell, once the DNA molecule coding for a biliverdin reductase protein or polypeptide, or fragment or variant thereof, has been ligated to its appropriate regulatory regions using well known molecular cloning techniques, it can then be introduced into a suitable vector or otherwise introduced directly into a host cell using transformation protocols well known in the art (Sambrook et al., Molecular Cloning: A Laboratory Manual, Second Edition, Cold Spring Harbor Press, N.Y. (1989), which is hereby incorporated by reference).

The recombinant molecule can be introduced into host cells via transformation, particularly transduction, conjugation, mobilization, or electroporation. Suitable host cells include, but are not limited to, bacteria, virus, yeast, mammalian cells, insect, plant, and the like. The host cells, when grown in an appropriate medium, are capable of expressing the biliverdin reductase, or fragment or variant thereof, which can then be isolated therefrom and, if necessary, purified. The biliverdin reductase, or fragment or variant thereof, is preferably produced in purified form (preferably at least about 60%, more preferably 80%, pure) by conventional techniques.

A further aspect of the present invention relates to an antisense nucleic acid molecule capable of hybridizing with an RNA transcript coding for BVR. Basically, the antisense nucleic acid is expressed from a transgene which is prepared by ligation of a DNA molecule, coding for BVR, or a fragment or variant thereof, into an expression vector in reverse orientation with respect to its promoter and 3′ regulatory sequences. Upon transcription of the DNA molecule, the resulting RNA molecule will be complementary to the mRNA transcript coding for the actual protein or polypeptide product. Ligation of DNA molecules in reverse orientation can be performed according to known techniques which are standard in the art.

Such antisense nucleic acid molecules of the invention may be used in gene therapy to treat or prevent various disorders. For a discussion of the regulation of gene expression using anti-sense genes, see Weintraub et al., Reviews—Trends in Genetics, 1(1) (1986), which is hereby incorporated by reference. As discussed infra, recombinant molecules including an antisense sequence or oligonucleotide fragment thereof, may be directly introduced into cells of tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes.

By virtue of the surprising discovery that BVR is not simply a house-keeping enzyme, but instead possesses kinase activity and capability of modulating the activity of other kinases, the present invention concerns a number of therapeutic uses for BVR or fragments or variants thereof.

One aspect of the present invention relates to a method of regulating protein kinase activity which is carried out by contacting a protein kinase with biliverdin reductase, or a fragment or variant thereof, under conditions effective to regulate activity of the protein kinase. BVR or the fragment or variant thereof can either enhance kinase activity or inhibit kinase activity. Because of its diverse capacity for phosphorylation, BVR is capable of regulating a number of diverse kinases including, without limitation, protein kinase A (“PKA”) and protein kinase C (“PKC”).

There are two types of PKA, type I (PKA-I) and type II (PKA-II), both of which share a common C subunit but each containing distinct R subunits, RI and RII, respectively (Beebe et al., In The Enzymes: Control by Phosphorylation, 17 (A):43-111 (Academic, New York) (1986), which is hereby incorporated by reference). The R subunit isoforms differ in tissue distribution Oyen et al., FEBS Lett. 229:391-394 (1988); Clegg et al., Proc. Natl. Acad. Sci. USA 85:3703-3707 (1988), which are hereby incorporated by reference) and in biochemical properties (Beebe et al., In The Enzymes: Control by Phosphorylation, 17(A):43-111 (Academic Press, NY)(1986); Cadd et al., J. Biol. Chem. 265:19502-19506 (1990), which are hereby incorporated by reference).

As an inhibitor of PKA, BVR or fragments or variants thereof which act as PKA inhibitor are useful in the treatment of conditions in which PKA has a demonstrated role in disease pathology. The primary condition recognized in the art is cancer.

Increased PKA expression levels, and specifically the expression levels of RIα, are associated with cancer cell lines, primary tumors, and transformation with the Ki-ras oncogene or transforming growth factor-α (Lohmann, In Advances in Cyclic Nucleotide and Protein Phosphorylation Research, 18: 63-117 (Raven, New York) (1984); Cho-Chung, Cancer Res. 50:7093-7100 (1990), which are hereby incorporated by reference); whereas, decreased PKA expression levels, and specifically the expression levels of RIα, are with growth inhibition induced by site-selective cAMP analogs in a broad spectrum of human cancer cell lines (Cho-Chung, Cancer Res. 50:7093-7100 (1990), which is hereby incorporated by reference). It has also been determined that the expression of RI/PKA-I and RII/PKA-II has an inverse relationship during ontogenic development and cell differentiation (Lohmann, In Advances in Cyclic Nucleotide and Protein Phosphorylation Research, 18: 63-117 (Raven, New York) (1984); Cho-Chung, Cancer Res. 50:7093-7100 (1990), which are hereby incorporated by reference). The RI alpha subunit of PKA has thus been hypothesized to be an oncogenic growth-inducing protein whose constitutive expression disrupts normal oncogenic processes, resulting in a pathogenic outgrowth, such as malignancy (Nesterova et al., Nature Medicine 1:528-533 (1995), which is hereby incorporated by reference).

PKC consists of a family of closely related enzymes that function as serine/threonine kinases. PKC plays an important role in cell-cell signaling, gene expression, and in the control of cell differentiation and growth. At present, there are currently at least ten known isozymes of PKC that differ in their tissue distribution, enzymatic specificity, and regulation (Nishizuka, Annu. Rev. Biochem. 58:31-44 (1989); Nishizuka, Science 258:607-614 (1992), which are hereby incorporated by reference).

PKC isozymes are single polypeptide chains ranging from 592 to 737 amino acids in length. The isozymes contain a regulatory domain and a catalytic domain connected by a linker peptide. The regulatory and catalytic domains can be further subdivided into constant and variable regions. The catalytic domain of PKC is very similar to that seen in other protein kinases while the regulatory domain is unique to the PKC isozymes. The PKC isozymes demonstrate between 40-80% homology at the amino acid level among the group. However, the homology of a single isozyme between different species is generally greater than 97%.

PKC is a membrane-associated enzyme that is allosterically regulated by a number of factors, including membrane phospholipids, calcium, and certain membrane lipids such as diacylglycerols that are liberated in response to the activities of phospholipases (Bell et al., J. Biol. Chem. 266:4661-4664 (1991); Nishizuka, Science 258:607-614 (1992), which are hereby incorporated by reference). The PKC isozymes alpha, beta-1, beta-2, and gamma require membrane phospholipid, calcium and diacylglycerol/phorbol esters for full activation. The delta,. epsilon, eta, and theta forms of PKC are calcium-independent in their mode of activation. The zeta and lambda forms of PKC are independent of both calcium and diacylglycerol and are believed to require only membrane phospholipid for their activation.

Fragments or variants of BVR which act as a PKC inhibitor are useful in the treatment of conditions in which PKC has a demonstrated role in disease pathology. Conditions recognized in the art include: diabetes mellitus and its complications, ischemia, inflammation, central nervous system disorders, cardiovascular disease, Alzheimer's disease, dermatological disease and cancer.

Protein kinase C inhibitors have been shown to block inflammatory responses such as neutrophil oxidative burst, CD3 down-regulation in T-lymphocytes, and phorbol-induced paw edema (Twoemy et al., Biochem. Biophys. Res. Commun. 171:1087-1092 (1990); Mulqueen et al., Agents Actions 37: 85-89 (1992), which are hereby incorporated by reference). Accordingly, as inhibitors of PKC, the present compounds are useful in treating inflammation.

Protein kinase C activity plays a central role in the functioning of the central nervous system (Huang, Trends Neurosci. 12:425-432 (1989), which is hereby incorporated by reference). In addition, protein kinase C inhibitors have been shown to prevent the damage seen in focal and central ischemic brain injury and brain edema (Hara et al., J. Cereb. Blood Flow Metab. 10:646-653 (1990); Shibata et al., Brain Res. 594:290-294 (1992), which are hereby incorporated by reference). Recently, protein kinase C has been determined to be implicated in Alzheimer's disease (Shimohama et al., Neurology 43:1407-1413 (1993), which is hereby incorporated by reference). Accordingly, the compounds of the present invention are useful in treating Alzheimer's disease and stroke/ischemic brain injury.

Protein kinase C activity has long been associated with cell growth, tumor promotion and cancer (Rotenberg et al., Biochem. Mol. Aspects Sel. Cancer 1:25-73 (1991); Ahmad et al., Mol. Pharmacol. 43:858-862 (1993), which are hereby incorporated by reference). It is known that inhibitors of protein kinase C are effective in preventing tumor growth in animals (Meyer et al., Int. J. Cancer 43:851-856 (1989); Akinagaka et al., Cancer Res. 51:4888-4892 (1991), which are hereby incorporated by reference). The PKC inhibitors of the present invention can also act in conjunction with other chemotherapeutic agents.

Protein kinase C activity also plays an important role in cardiovascular disease. Increased protein kinase C activity in the vasculature has been shown to cause increased vasoconstriction and hypertension. A known protein kinase C inhibitor prevented this increase (Bilder et al., J. Pharmacol. Exp. Ther. 252:526-530 (1990), which is hereby incorporated by reference). Because protein kinase C inhibitors demonstrate inhibition of the neutrophil oxidative burst, protein kinase C inhibitors are also useful in treating cardiovascular ischemia and improving cardiac function following ischemia (Muid et al., FEBS Lett. 293:169-172 (1990); Sonoki et al., Kokyu-To Junkan 37: 669-674 (1989), which are hereby incorporated by reference).

The role of protein kinase C in platelet function has also been investigated, with elevated protein kinase C levels being correlated with increased response to agonists (Bastyr et al., Diabetes 42(Suppl. 1):97A (1993), which is hereby incorporated by reference). PKC has been implicated in the biochemical pathway in the platelet-activity factor modulation of microvascular permeability (Kobayashi et al., Amer. Phys. Soc. H1214-H1220 (1994), which is hereby incorporated by reference). Potent protein kinase C inhibitors have been demonstrated to affect agonist-induced aggregation in platelets (Toullec et al., J. Biol. Chem. 266:15771-15781 (1991), which is hereby incorporated by reference). Protein kinase C inhibitors also block agonist-induced smooth muscle cell proliferation (Matsumoto et al., Biochem. Biophys. Res. Commun. 158:105-109 (1989), which is hereby incorporated by reference). Therefore, the present compounds are useful in treating cardiovascular disease, atherosclerosis, and restenosis.

Abnormal activity of protein kinase C has also been linked to dermatological disorders such as psoriasis (Horn et al., J. Invest. Dermatol. 88:220-222 (1987); Raynaud et al., Br. J. Dermatol. 124:542-546 (1991), which are hereby incorporated by reference). Psoriasis is characterized by abnormal proliferation of keratinocytes. Known protein kinase C inhibitors have been shown to inhibit keratinocyte proliferation in a manner that parallels their potency as PKC inhibitors (Hegemann et al., Arch. Dermatol. Res. 283:456-460 (1991); Bollag et al., J. Invest. Dermatol. 100:240-246 (1993), which are hereby incorporated by reference). Accordingly, the compounds as inhibitors of PKC are useful in treating psoriasis.

Protein kinase C has been linked to several different aspects of diabetes. Excessive activity of protein kinase C has been linked to insulin signaling defects and therefore to the insulin resistance seen in Type II diabetes (Karasik et al., J. Biol. Chem. 265:10226-10231 (1990); Chen et al., Trans. Assoc. Am. Physicians 104:206-212 (1991); Chin et al., J. Biol. Chem. 268:6338-6347 (1993), which are hereby incorporated by reference). In addition, studies have demonstrated a marked increase in protein kinase C activity in tissues known to be susceptible to diabetic complications when exposed to hyperglycemic conditions (Lee et al., J. Clin. Invest. 83:90-94 (1989); Lee et al., Proc. Natl. Acad. Sci. USA 86:5141-5145 (1989); Craven et al., J. Clin. Invest. 83:1667-1675 (1989); Wolfet al., J. Clin. Invest. 87:31-38 (1991); and Tesfamariam et al., J. Clin. Invest. 87:1643-1648 (1991), which are hereby incorporated by reference).

PKC has also been implicated in HO-1 phosphorylation (Snyder et al., Brain Res. Brain Res. Rev. 26:167-175 (1998), which is hereby incorporated by reference). Therefore, it is reasonable to believe that BVR modulation of PKC activity can also affect the upstream heme oxygenase pathway and carbon monoxide production. As such, BVR is again expected to have practical use in modulating all diseases or disorders in which HO administration is beneficial including, without limitation, as an antiinflammatory (see U.S. Pat. No. 6,066,333 to Willis et al., which is hereby incorporated by reference), allograft rejection, carcinogenesis, neuroendocrine functions, and neuronal signaling (see review by Lane et al., The Sciences 24-29 (September/October 1998), which is hereby incorporated by reference).

One preferred inhibitor of PKC activity is a polypeptide fragment of BVR comprising a C-terminal fragment of rBVR or HBVR. More specifically, the polypeptide fragment comprises aa 288-295 of rBVR (SEQ. ID. No. 19) as follows:

QKLCHQKK

or aa 289-296 of hBVR (SEQ. ID. No. 35) as follows:

QKYCCSRK

As an enhancer or stimulator of PKC activity, BVR or fragments or variants thereof which act as a PKC enhancer are also useful in promoting desirable PKC regulated activities. For example, due to the its involvement in cell proliferation and differentiation, it may be desirable in certain circumstances to induce PKC-mediated cell proliferation. Such conditions may involve in vitro growth proliferation for the study of tumor cells or for the production of desired cellular products.

One preferred enhancer of PKC activity is a fragment of BVR comprising an internal fragment of rBVR or hBVR. More specifically, the polypeptide fragment comprises aa 274-280 of rBVR (SEQ. ID. No. 18) as follows:

KKRIMHC

or aa 275-281 of hBVR (SEQ. ID. No. 34) as follows:

KKRILHC

To modulate kinase activity, BVR or fragments or variants thereof need to contact the kinase. Such contacting may occur in an in vitro assay system of the type described infra. However, when modulating kinase activity in a cell, the contacting is carried out in the cell. The cell can be any mammalian cell, but preferably a human cell, which is either in vitro or in vivo.

BVR is a serine-, threonine-, and tyrosine-kinase which is capable of both autophosphorylation and phosphorylation of other proteins. Thus, BVR, or fragments or variants thereof, are capable of not only modulating protein kinase to affect cell differentiation, growth, or signaling, but also directly affecting cell differentiation, growth, or signaling. Therefore, another aspect of the present invention relates to a method of regulating cell differentiation, growth, or signaling which is carried out by contacting a cell with biliverdin reductase, or fragment or variant thereof, under conditions effective to regulate cell differentiation, growth, or signaling.

Tyrosine kinases form an important class of molecules involved in the regulation of growth and differentiation (Ullrich et al., Cell 61:203-212 (1990), which is hereby incorporated by reference). One mode of proof for this role came from the identification of receptors which bind known soluble growth factors. The receptors for epidermal growth factor (Carpenter et al., J. Biol. Chem. 265:7709-7712 (1990), which is hereby incorporated by reference), platelet derived growth factor (PDGF) (Williams, Science 243:1564-1570 (1989), which is hereby incorporated by reference), and colony stimulating factor-1 (CSF-1) (Yeung et al., Proc. Natl. Acad. Sci. USA 84:1268-1271 (1987), which is hereby incorporated by reference) were all shown to be transmembrane molecules with the cytoplasmic regions encoding a tyrosine kinase catalytic domain. The CSF-1 receptor is homologous to the PDGF receptor in both the catalytic and extracellular domains (Ullrich et al., Cell 61:203-212 (1990); Hanks et al., Science 241:42-52 (1988), which are hereby incorporated by reference). The extra cellular domain of these proteins is distinguished from other tyrosine kinases by the presence of immunoglobulin-like repeats (Ullrich et al., Cell 61:203-212 (1990); Yarden et al., Ann. Rev. Biochem. 57:443-478 (1988), which are hereby incorporated by reference). Based on structural properties of the kinase domain, the c-kit protein was identified as another member of this family (Yarden et al., EMBO J. 6:3341-3351(1987), which is hereby incorporated by reference). The c-kit gene locus appears to underpin the defects in the congenitally anaemic W/W mouse (Chabot et al., Nature 335:88-89 (1988); Geissler et al., Cell 55:185-192 (1988); Nocka et al., Genes Dev. 3:816-826 (1989), which are hereby incorporated by reference). The ligand has now been identified (Williams et al., Cell 63:167-174 (1990); Zsebo et al., Cell 63:213-244 (1990); Huang et al., Cell 63:225-233 (1990); Copeland et al., Cell 63:175-183 (1990), which are hereby incorporated by reference) and shown to be encoded by the SI locus. The locus is abnormal in the Steel mouse (Bennett, Morphol. 98:199-233 (1956), which is hereby incorporated by reference) which has identical defects to the W/W mouse but encodes a normal c-kit gene.

The other line of evidence for a critical role of tyrosine kinase proteins in growth control came from the study of viral oncogenes (Bishop, Ann. Rev. Biochem. 52:301-354 (1983); Hunter et al., Ann. Rev. Biochem. 54:897-930 (1985), which are hereby incorporated by reference). These genes were shown to be directly involved in growth dysregulation by observations of a change in cell growth following introduction of DNA encoding these genes into fibroblasts. All oncogenes have been shown to have close cellular homologues (proto-oncogenes). One of the first identified oncogenes was v-src, the cellular homologue (c-src) is the prototypical representative of the family of cytoplasmic tyrosine kinases which, following myristylation, become associated with the inner leaf of the cell membrane (Resh, Oncogenes 1437-1444 (1990), which is hereby incorporated by reference). Within the haemopoietic system a number of lineage-restricted src-like kinases have been defined (Eiseman et al., Cancer Cells 2:303-310 (1990), which is hereby incorporated by reference).

Detailed analysis of the amino acid sequences of these proteins has revealed conserved structural motifs within the catalytic domains (Hanks et al., Science 241:42-52 (1988), which is hereby incorporated by reference). Both tyrosine and serine-threonine kinases have a consensus GXGXXG sequence (SEQ. ID. No. 7) which is found in many nucleotide binding proteins (Hanks et al., Science 241:42-52 (1988), which is hereby incorporated by reference). Other conserved sequence motifs are shared by both types of kinase while others are specific for the tyrosine or the threonine-serine kinase subgroups (Hanks et al., Science 241:42-52 (1988), which is hereby incorporated by reference). The tyrosine kinases, while having regions of sequence conservation specific to this family, can be further subdivided according to the structural features of the regions 5′ to the catalytic domain (Yeung et al., Proc. Natl. Acad. Sci. USA 84:1268-1271 (1987); Hanks et al., Science 241:42-52 (1988); Yarden et al., Ann. Rev. Biochem. 57:443-478 (1988); Yarden et al., EMBO J. 6:3341-3351 (1987), which are hereby incorporated by reference). BVR exhibits many of the same general characteristics as previously known tyrosine kinases. Therefore, BVR is expected to share similar utilities in regulating cellular growth and differentiation as other tyrosine kinases.

Serine/threonine kinases also form an important class of molecules involved in the regulation of a number of cellular activities, including cellular responses to stress mechanisms, cellular differentiation, and intracellular signaling, among others.

Cellular response mechanisms to stress are fundamentally important to the human immune system. Stress responses represent carefully devised cellular defense mechanisms which were developed at an early point during evolution; evidenced by the fact that biomolecules implicated in stress response exhibit remarkable similarity across the animal kingdom (Welch et al., The Stress Response and the Immune System. Inflammation: Basic Principles and Clinical Correlates, Raven Press, Gallin, J. I., et al., Eds., Second Edition, 41:841(1992), which is hereby incorporated by reference).

Lymphocyte activation, homing, resistance to target cell lysis, tumor antigenicity, regulation of proto-oncogene transcription, and immune surveillance are examples of immunologic functions that appear to be mediated or modulated by stress activated signal transduction molecules (Siegelman et al., Science 231:823 (1986); Kusher et al., J. Immunol. 145:2925 (1990); Ullrich et al., PNAS 83:3121 (1986); Colotta et al., Biochem. Biophys. Res. Commun. 168:1013 (1990); Haire et al., J. Cell Biol. 106:883 (1988); Born et al., Immunol. T., 11:40 (1990), which are hereby incorporated by reference). The number of preactivated and MHC class II-restricted autoreactive T-lymphocytes in peripheral blood of patients with rheumatoid arthritis, for example, dramatically increases relative to the levels in healthy individuals. Similarly, peripheral blood T-lymphocytes from patients with inflammatory arthritis proliferate strongly in the absence of exogenous antigen or mitogen (Welch et al., The Stress Response and the Immune System. Inflammation: Basic Principles and Clinical Correlates, Raven Press, Gallin, J. I., et al., Eds., Second Edition, Chapter 41, 841 (1992), which is hereby incorporated by reference). Moreover, synovitis has been shown to result in the generation of oxygen-derived free radicals that act to perpetuate tissue damage (Blake et al., Lancet 2:2889 (1989), which is hereby incorporated by reference).

The control of hematopoiesis is a highly regulated process that responds to a number of physiological stimuli in the human body. Differentiation, proliferation, growth arrest, or apoptosis of blood cells depends on the presence of appropriate cytokines and their receptors, as well as the corresponding cellular signal transduction cascades (Hu et al., Genes & Development, 10:2251(1996), which is hereby incorporated by reference). Generation of mature leukocytes, for instance, is a highly regulated process which responds to various environmental and physiological stimuli. Cytokines cause cell proliferation, differentiation or elimination, each of these processes being dependent on the presence of appropriate cytokine receptors and the corresponding signal transduction elements. Moreover, the stimulation of quiescent B- and T-lymphocytes occur via antigen receptors which exhibit remarkable homology to cytokine receptors (Grunicke, Signal Transduction Mechanisms in Cancer, Springer-Verlag (1995); Suchard et al., J. Immunol. 158:4961 (1997), which are hereby incorporated by reference).

Distinct signaling cassettes, each containing a central cascade of kinases, respond to a variety of positive and negative extracellular stimuli, leading to changes in transcription factor activity and posttranslational protein modifications in mammalian cells (Kiefer et al., EMBO J. 5(24):7013 (1996), which is hereby incorporated by reference). One such protein kinase cascade, known as the mitogen-activated protein kinase (MAPK) cascade, is activated as an early event in the response of leukocytes to various stimuli. Stimulation of this pathway has been observed during growth factor-induced DNA synthesis, differentiation, secretion, and metabolism. The MAPK pathway has a critical role in the transduction of receptor-generated signals from the membrane to the cytoplasm and nucleus (Graves et al., Ann. New York Acad. Sci. 766:320 (1995), which is hereby incorporated by reference). It has been established that sustained activation of the MAPK cascade is not only required, but it is sufficient to trigger the proliferation of some cells and the differentiation of others (Cohen, In Advances in Pharmacology, Academic Press, Hidaka, et al., Eds., Vol. 36, 15 (1996); Marshall, Cell 80:179 (1995), which are hereby incorporated by reference). Several interdependent biochemical pathways are activated following either stimulation of resting T-lymphocytes through the antigen receptor or stimulation of activated T-lymphocytes through the interleukin-2 (IL-2) receptor. Many of the events that occur after the engagement of either of these receptors are qualitatively similar, such as the activation of MAPK pathways and preexisting transcription factors, leading to the expression of specific growth-associated genes (Modiano et al., Ann. New York Acad. Sci. 766:134 (1995), which is hereby incorporated by reference).

Recent evidence suggests that cellular response to stress is controlled primarily through events occurring at the plasma membrane, overlapping significantly with those important in initiating mitogenic responses. The MAPK pathway has been shown to be essential for the mitogenic response in many systems (Qin et al., J.Cancer Res.Clin.Oncol. 120:519 (1994), which is hereby incorporated by reference). Moreover, due to the fact that most oncogenes encode growth factors, growth factor receptors, or elements of the intracellular postreceptor signal-transmission machinery, it is becoming increasingly apparent that growth factor signal transduction pathways are subject to an elaborate network of positive and negative cross-regulatory inputs from other transformation-related pathways (Grunicke, Signal Transduction Mechanisms in Cancer, Springer-Verlag (1995), which is hereby incorporated by reference). The hierarchical organization of the MAPK cascade makes integral protein kinase members particularly good targets for such “cross-talk” (Graves et al., Ann. New York Acad. Sci. 766:320 (1995), which is hereby incorporated by reference).

Initial triggers for inflammation include physical and chemical agents, bacterial and viral infections, as well as exposure to antigens, superantigens or allergens, all of which have the potential to generate Reactive Oxygen Species (ROS) and to thereby activate second messenger signal transduction molecules (Storz et al., In Stress-Inducible Cellular Responses, Feige et al., Eds., Birkhauser Verlag (1996), which is hereby incorporated by reference). Reactive oxygen radicals, via damage to many cellular components including DNA, can cause cell death or, if less severe, cell cycle arrest at growth-phase checkpoints.

Stress damage not only activates checkpoint controls but also activates protein kinases, including the stress activated protein kinases (SAPKs), c-Raf-1 and ERKs, which are integral components of cytoplasmic signal transduction (i.e., MAPK) cascades (Pombo et al., EMBO J. 15(17):4537 (1996); Russo et al., J.Biol. Chem. 270:29386 (1995), which are hereby incorporated by reference). Considering that stress has also been implicated in oxidant injury, atherosclerosis, neurogenerative processes, and aging, elucidation of the components of mammalian stress-induced pathways should provide more specific targets that can be exploited therapeutically. Holbrook et al., In Stress Inducible Cellular Responses, 273, Feige et al., Eds., Birkhauser Verlag (1996), which is hereby incorporated by reference).

Evidence has demonstrated that MAPK and stress activated protein kinase (SAPK) signal transduction pathways are responsible for triggering biological effects across a wide variety of pathophysiological conditions including conditions manifested by dysfunctional leukocytes, T-lymphocytes, acute and chronic inflammatory disease, auto-immune disorders, rheumatoid arthritis, osteoarthritis, transplant rejection, macrophage regulation, endothelial cell regulation, angiogenesis, atherosclerosis, fibroblasts regulation, pathological fibrosis, asthma, allergic response, ARDS, atheroma, osteoarthritis, heart failure, cancer, diabetes, obesity, cachexia, Alzheimer's disease, sepsis, and neurodegeneration. As MAP kinases play a central role in signaling events which mediate cellular response to stress, their inactivation is key to the attenuation of the response (Holbrook et al., In Stress Inducible Cellular Responses, 273, Feige et al., Eds., Birkhauser Verlag (1996), which is hereby incorporated by reference).

Integral members of cellular signaling pathways as targets for therapeutic development, for example, have been the subject to several reviews (Levitzki, Eur. J. Biochem. 226:1 (1994); Powis, In New Molecular Targets for Cancer Chemotherapy, Workman et al., CRC Press, Boca Raton Fla. (1994), which are hereby incorporated by reference).

In addition to its role in directly regulating kinases, BVR also acts as an intracellular signaling molecule, most likely via its association with cyclic guanosine monophosphate (“cGMP”). That BVR also associates with G protein-coupled receptors is supported by the presence of the hydrophobic domain characterized by FXVVVV (SEQ. ID. No. 6). This domain is conserved among membrane associated proteins such as G protein coupled receptor ion transporters, surfactants, other receptors such as vascular endothelial growth factor receptor and CD30 receptor, chemokin receptor, somatostatin receptor, etc. Thus, BVR-cGMP may regulate cGMP-activated protein kinase (“PKG”). Moreover, because BVR lies upstream of the heme oxygenase pathway (which yields carbon monoxide) and carbon monoxide may be involved in generation of cGMP, it is believed that BVR can regulate, both directly and indirectly, cGMP signaling and PKG activity. Both clinical application and research studies have demonstrated that stimulation of the cGMP/PKG pathway is useful for treatment of: (i) heart disease including stable angina pectoris, unstable angina, myocardial infarction, and myocardial failure associated with myocardial ischemia, atherosclerosis, vascular hypertrophy, and thrombosis (Cooe et al., Annu. Rev. Med. 48:489-509 (1997); Thadani, Cardiovasc. Drugs 10:735 (1997), which are hereby incorporated by reference); (ii) hypertension (Cooe et al., Annu. Rev. Med. 48:489-509 (1997), which is hereby incorporated by reference); (iii) stroke (Samdani et al., Stroke 28:1283-1288 (1997), which is hereby incorporated by reference); (iv) primary pulmonary hypertension, chronic obstructive pulmonary disease, and adult respiratory distress syndrome (Adnot et al., Thorax 51:762-764 (1996); Marriott et al., Schweiz Med. Wochenschr. 127:709-714 (1997), which are hereby incorporated by reference); (v) microvascular functional abnormalities in diabetes that link insulin-resistance to hypertension, thrombosis, and atherosclerosis (Tooke et al., Diabetes Res. Clin. Pract. 31 Suppl:S127-S132 (1996); Baron, J. Investig. Med. 44:406-412 (1996), which are hereby incorporated by reference); (vi) hemostatic irregularities of glomerular vascular and tubular function with consequences for development of hypertension (Kone et al., Am. J. Physiol. 10:F561-578 (1997); Am. J. Hypertens. 10:129-140 (1997), which are hereby incorporated by reference); (vii) microvascular irregularities in the liver with consequences for biliary transport and tissue regeneration (Suematsu, et al., Cardiovasc. Res. 32:679-686 (1996), which is hereby incorporated by reference); (viii) disorders of bladder function and reflex relaxation for micturition (Andersson, Curr. Opin. Obstet. Gynecol. 8:361-365 (1996), which is hereby incorporated by reference); (ix) disorders of neurotransmitter release, neuron morphogenesis, synaptic plasticity, and neuroendrocrine regulation (Dawson et al., Neurochem. Int. 29:97-110 (1996); Brann et al., Neuroendocrinology 65:385-395 (1997), which are hereby incorporated by reference); (x) regional pain including migraine headaches (Mashimo et al., J. Clin. Pharmacol. 37:330-335 (1997); Packard et al., Mar. 37:142-152 (1997), which are hereby incorporated by reference); (xi) gastrointestinal protection from non-steroidal anti-inflammatory drugs (Rishi et al., Indian J. Physiol. Pharmacol. 40:377-379 (1996), which is hereby incorporated by reference); (xii) benign anal disease (Gorfine, Dis. Colon Rectum 38:453-456 (1995), which is hereby incorporated by reference); (xiii) impotence (Andersson et al., World J. Urol. 15:14-20 (1997), which is hereby incorporated by reference); (xiv) regulation of tissue free radical injury (Rubbo et al., Chem. Res. Toxicol. 9:809-820 (1996), which is hereby incorporated by reference); (xv) inhibition of tumor growth, tumor apoptosis, angiogenesis, and metastasis (Pipili-Synetos et al., Br. J. Pharmacol. 116:1829-1834 (1995); Xie et al., J. Leukoc. Biol. 59:797-803 (1996), which are hereby incorporated by reference); and (xvi) stimulation of wound healing including cuts, tendon injury, and thermal injury (Schaffer et al., J. Surg. Res. 63:237-240 (1996); Murrell et al., Inflamm. Res. 46:19-27 (1997); Carter et al., Biochem. J. 304(Pt 1):201-04 (1994), which are hereby incorporated by reference).

The methods and compositions of the present invention contemplate the use BVR or fragments or variants thereof either to associate directly with cGMP or indirectly generate cGMP, i.e., through HO, to modulate cGMP-activated protein kinase (PKG) activity. This should affect a variety of physiological processes depending upon the treated tissue, including but not limited to mediation of blood vessel relaxation, mediation of neurotransmission, mediation of neuronal differentiation, regulation of free-radical injury, and mediation of melanogenesis.

By virtue of BVR's role in modulating activity of protein kinases as well as its ability to act as a serine-, threonine-, and tyrosine-kinase, BVR or fragments or variants thereof, can be used to treat cellular dysfunction or disease. Thus, a further aspect of the present invention relates to a method of treating cellular dysfunction or disease which is carried out by contacting a dysfunctional or diseased cell with biliverdin reductase or fragment or variant thereof under conditions effective to treat or immolate the dysfunctional or diseased cell.

According to various aspects of the present invention, it may also be desirable in several therapies to modify the expression levels or intracellular concentration of other enzymes or substrates. This can be achieved by contacting the cell with the enzyme or substrate or antisense RNA which can inhibit expression of such enzyme or substrate.

One such enzyme is poly(ADP-ribose) polymerase, which can be introduced or heterologously expressed within the treated cell. For example, when treating or immolating cancer cells, it is desirable to increase the intracellular concentration of poly(ADP-ribose) polymerase. Without being bound by theory, it is believed that the NADH-dependent activity of BVR or fragments or variants thereof, coupled with poly(ADP-ribose) polymerase activity, will deplete cancer cells of ATP reserves, thereby immolating the treated cell. In contrast, when treating stroke/ischemic event (i.e., oxidative stress) to prevent further cell degradation (i.e., cell death), it is desirable to reduce the expression or intracellular concentration of poly(ADP-ribose) polymerase. This can be achieved by contacting the cell with antisense RNA capable of hybridizing to RNA transcripts coding for poly(ADP-ribose) polymerase, thereby preventing their expression. The antisense RNA is preferably introduced into or expressed in the treated cell. Preparation of DNA molecules coding for antisense mRNA can be prepared as described above.

Human poly(ADP-ribose) polymerase has an amino acid sequence according to SEQ. ID. No. 20 as follows:

| Met | Ala | Glu | Ser | Ser | Asp | Lys | Leu | Tyr | Arg | Val | Glu | Tyr | Ala | Lys | Ser |
|---|---|---|---|---|---|---|---|---|---|---|---|---|---|---|---|
| 1 | | | | 5 | | | | | 10 | | | | | 15 | |
| Gly | Arg | Ala | Ser | Cys | Lys | Lys | Cys | Ser | Glu | Ser | Ile | Pro | Lys | Asp | Ser |
| | | | 20 | | | | | 25 | | | | | 30 | | |
| Leu | Arg | Met | Ala | Ile | Met | Val | Gln | Ser | Pro | Met | Phe | Asp | Gly | Lys | Val |
| | | 35 | | | | | 40 | | | | | 45 | | | |
| Pro | His | Trp | Tyr | His | Phe | Ser | Cys | Phe | Trp | Lys | Val | Gly | His | Ser | Ile |
| | 50 | | | | | 55 | | | | | 60 | | | | |
| Arg | His | Pro | Asp | Val | Glu | Val | Asp | Gly | Phe | Ser | Glu | Leu | Arg | Trp | Asp |
| 65 | | | | | 70 | | | | | 75 | | | | | 80 |
| Asp | Gln | Gln | Lys | Val | Lys | Lys | Thr | Ala | Glu | Ala | Gly | Gly | Val | Thr | Gly |
| | | | | 85 | | | | | 90 | | | | | 95 | |
| Lys | Gly | Gln | Asp | Gly | Ile | Gly | Ser | Lys | Ala | Glu | Lys | Thr | Leu | Gly | Asp |
| | | | 100 | | | | | 105 | | | | | 110 | | |
| Phe | Ala | Ala | Glu | Tyr | Ala | Lys | Ser | Asn | Arg | Ser | Thr | Cys | Lys | Gly | Cys |
| | | 115 | | | | | 120 | | | | | 125 | | | |
| Met | Glu | Lys | Ile | Glu | Lys | Gly | Gln | Val | Arg | Leu | Ser | Lys | Lys | Met | Val |
| | 130 | | | | | 135 | | | | | 140 | | | | |
| Asp | Pro | Glu | Lys | Pro | Gln | Leu | Gly | Met | Ile | Asp | Arg | Trp | Tyr | His | Pro |
| 145 | | | | | 150 | | | | | 155 | | | | | 160 |
| Gly | Cys | Phe | Val | Lys | Asn | Arg | Glu | Glu | Leu | Gly | Phe | Arg | Pro | Glu | Tyr |
| | | | | 165 | | | | | 170 | | | | | 175 | |
| Ser | Ala | Ser | Gln | Leu | Lys | Gly | Phe | Ser | Leu | Leu | Ala | Thr | Glu | Asp | Lys |
| | | | 180 | | | | | 185 | | | | | 190 | | |
| Glu | Ala | Leu | Lys | Lys | Gln | Leu | Pro | Gly | Val | Lys | Ser | Glu | Gly | Lys | Arg |
| | | 195 | | | | | 200 | | | | | 205 | | | |
| Lys | Gly | Asp | Glu | Val | Asp | Gly | Val | Asp | Glu | Val | Ala | Lys | Lys | Lys | Ser |
| | 210 | | | | | 215 | | | | | 220 | | | | |
| Lys | Lys | Glu | Lys | Asp | Lys | Asp | Ser | Lys | Leu | Glu | Lys | Ala | Leu | Lys | Ala |
| 225 | | | | | 230 | | | | | 235 | | | | | 240 |
| Gln | Asn | Asp | Leu | Ile | Trp | Asn | Ile | Lys | Asp | Glu | Leu | Lys | Lys | Val | Cys |
| | | | | 245 | | | | | 250 | | | | | 255 | |
| Ser | Thr | Asn | Asp | Leu | Lys | Glu | Leu | Leu | Ile | Phe | Asn | Lys | Gln | Gln | Val |
| | | | 260 | | | | | 265 | | | | | 270 | | |
| Pro | Ser | Gly | Glu | Ser | Ala | Ile | Leu | Asp | Arg | Val | Ala | Asp | Gly | Met | Val |
| | | 275 | | | | | 280 | | | | | 285 | | | |
| Phe | Gly | Ala | Leu | Leu | Pro | Cys | Glu | Glu | Cys | Ser | Gly | Gln | Leu | Val | Phe |
| | 290 | | | | | 295 | | | | | 300 | | | | |
| Lys | Ser | Asp | Ala | Tyr | Tyr | Cys | Thr | Gly | Asp | Val | Thr | Ala | Trp | Thr | Lys |
| 305 | | | | | 310 | | | | | 315 | | | | | 320 |
| Cys | Met | Val | Lys | Thr | Gln | Thr | Pro | Asn | Arg | Lys | Glu | Trp | Val | Thr | Pro |
| | | | | 325 | | | | | 330 | | | | | 335 | |
| Lys | Glu | Phe | Arg | Glu | Ile | Ser | Tyr | Leu | Lys | Lys | Leu | Lys | Val | Lys | Lys |
| | | | 340 | | | | | 345 | | | | | 350 | | |
| Gln | Asp | Arg | Ile | Phe | Pro | Pro | Glu | Thr | Ser | Ala | Ser | Val | Ala | Ala | Thr |
| | | 355 | | | | | 360 | | | | | 365 | | | |
| Pro | Pro | Pro | Ser | Thr | Ala | Ser | Ala | Pro | Ala | Ala | Val | Asn | Ser | Ser | Ala |
| | 370 | | | | | 375 | | | | | 380 | | | | |
| Ser | Ala | Asp | Lys | Pro | Leu | Ser | Asn | Met | Lys | Ile | Leu | Thr | Leu | Gly | Lys |
| 385 | | | | | 390 | | | | | 395 | | | | | 400 |
| Leu | Ser | Arg | Asn | Lys | Asp | Glu | Val | Lys | Ala | Met | Ile | Glu | Lys | Leu | Gly |
| | | | | 405 | | | | | 410 | | | | | 415 | |
| Gly | Lys | Leu | Thr | Gly | Thr | Ala | Asn | Lys | Ala | Ser | Leu | Cys | Ile | Ser | Thr |
| | | | 420 | | | | | 425 | | | | | 430 | | |
| Lys | Lys | Glu | Val | Glu | Lys | Met | Asn | Lys | Lys | Met | Glu | Glu | Val | Lys | Glu |
| | | 435 | | | | | 440 | | | | | 445 | | | |
| Ala | Asn | Ile | Arg | Val | Val | Ser | Glu | Asp | Phe | Leu | Gln | Asp | Val | Ser | Ala |
| | 450 | | | | | 455 | | | | | 460 | | | | |
| Ser | Thr | Lys | Ser | Leu | Gln | Glu | Leu | Phe | Leu | Ala | His | Ile | Leu | Ser | Pro |
| 465 | | | | | 470 | | | | | 475 | | | | | 480 |
| Trp | Gly | Ala | Glu | Val | Lys | Ala | Glu | Pro | Val | Glu | Val | Val | Ala | Pro | Arg |
| | | | | 485 | | | | | 490 | | | | | 495 | |
| Gly | Lys | Ser | Gly | Ala | Ala | Leu | Ser | Lys | Lys | Ser | Lys | Gly | Gln | Val | Lys |
| | | | 500 | | | | | 505 | | | | | 510 | | |
| Glu | Glu | Gly | Ile | Asn | Lys | Ser | Glu | Lys | Arg | Met | Lys | Leu | Thr | Leu | Lys |
| | | 515 | | | | | 520 | | | | | 525 | | | |
| Gly | Gly | Ala | Ala | Val | Asp | Pro | Asp | Ser | Gly | Leu | Glu | His | Ser | Ala | His |
| | 530 | | | | | 535 | | | | | 540 | | | | |
| Val | Leu | Glu | Lys | Gly | Gly | Lys | Val | Phe | Ser | Ala | Thr | Leu | Gly | Leu | Val |
| 545 | | | | | 550 | | | | | 555 | | | | | 560 |
| Asp | Ile | Val | Lys | Gly | Thr | Asn | Ser | Tyr | Tyr | Lys | Leu | Gln | Leu | Leu | Glu |
| | | | | 565 | | | | | 570 | | | | | 575 | |
| Asp | Asp | Lys | Glu | Asn | Arg | Tyr | Trp | Ile | Phe | Arg | Ser | Trp | Gly | Arg | Val |
| | | | 580 | | | | | 585 | | | | | 590 | | |
| Gly | Thr | Val | Ile | Gly | Ser | Asn | Lys | Leu | Glu | Gln | Met | Pro | Ser | Lys | Glu |
| | | 595 | | | | | 600 | | | | | 605 | | | |
| Asp | Ala | Ile | Glu | His | Phe | Met | Lys | Leu | Tyr | Glu | Glu | Lys | Thr | Gly | Asn |
| | 610 | | | | | 615 | | | | | 620 | | | | |
| Ala | Trp | His | Ser | Lys | Asn | Phe | Thr | Lys | Tyr | Pro | Lys | Lys | Phe | Tyr | Pro |
| 625 | | | | | 630 | | | | | 635 | | | | | 640 |
| Leu | Glu | Ile | Asp | Tyr | Gly | Gln | Asp | Glu | Glu | Ala | Val | Lys | Lys | Leu | Thr |
| | | | | 645 | | | | | 650 | | | | | 655 | |
| Val | Asn | Pro | Gly | Thr | Lys | Ser | Lys | Leu | Pro | Lys | Pro | Val | Gln | Asp | Leu |
| | | | 660 | | | | | 665 | | | | | 670 | | |
| Ile | Lys | Met | Ile | Phe | Asp | Val | Glu | Ser | Met | Lys | Lys | Ala | Met | Val | Glu |
| | | 675 | | | | | 680 | | | | | 685 | | | |
| Tyr | Glu | Ile | Asp | Leu | Gln | Lys | Met | Pro | Leu | Gly | Lys | Leu | Ser | Lys | Arg |
| | 690 | | | | | 695 | | | | | 700 | | | | |
| Gln | Ile | Gln | Ala | Ala | Tyr | Ser | Ile | Leu | Ser | Glu | Val | Gln | Gln | Ala | Val |
| 705 | | | | | 710 | | | | | 715 | | | | | 720 |
| Ser | Gln | Gly | Ser | Ser | Asp | Ser | Gln | Ile | Leu | Asp | Leu | Ser | Asn | Arg | Phe |
| | | | | 725 | | | | | 730 | | | | | 735 | |
| Tyr | Thr | Leu | Ile | Pro | His | Asp | Phe | Gly | Met | Lys | Lys | Pro | Pro | Leu | Leu |
| | | | 740 | | | | | 745 | | | | | 750 | | |
| Asn | Asn | Ala | Asp | Ser | Val | Gln | Ala | Lys | Val | Glu | Met | Leu | Asp | Asn | Leu |
| | | 755 | | | | | 760 | | | | | 765 | | | |
| Leu | Asp | Ile | Glu | Val | Ala | Tyr | Ser | Leu | Leu | Arg | Gly | Gly | Ser | Asp | Asp |
| 770 | | | | | 775 | | | | | 780 | | | | | |
| Ser | Ser | Lys | Asp | Pro | Ile | Asp | Val | Asn | Tyr | Glu | Lys | Leu | Lys | Thr | Asp |
| 785 | | | | | 790 | | | | | 795 | | | | | 800 |
| Ile | Lys | Val | Val | Asp | Arg | Asp | Ser | Glu | Glu | Ala | Glu | Ile | Ile | Arg | Lys |
| | | | | 805 | | | | | 810 | | | | | 815 | |
| Tyr | Val | Lys | Asn | Thr | His | Ala | Thr | Thr | His | Asn | Ala | Tyr | Asp | Leu | Glu |
| | | | 820 | | | | | 825 | | | | | 830 | | |
| Val | Ile | Asp | Ile | Phe | Lys | Ile | Glu | Arg | Glu | Gly | Glu | Cys | Gln | Arg | Tyr |
| | | 835 | | | | | 840 | | | | | 845 | | | |
| Lys | Pro | Phe | Lys | Gln | Leu | His | Asn | Arg | Arg | Leu | Leu | Trp | His | Gly | Ser |
| | 850 | | | | | 855 | | | | | 860 | | | | |
| Arg | Thr | Thr | Asn | Phe | Ala | Gly | Ile | Leu | Ser | Gln | Gly | Leu | Arg | Ile | Ala |
| 865 | | | | | 870 | | | | | 875 | | | | | 880 |
| Pro | Pro | Glu | Ala | Pro | Val | Thr | Gly | Tyr | Met | Phe | Gly | Lys | Gly | Ile | Tyr |
| | | | | 885 | | | | | 890 | | | | | 895 | |
| Phe | Ala | Asp | Met | Val | Ser | Lys | Ser | Ala | Asn | Tyr | Cys | His | Thr | Ser | Gln |
| | | | 900 | | | | | 905 | | | | | 910 | | |
| Gly | Asp | Pro | Ile | Gly | Leu | Ile | Leu | Leu | Gly | Glu | Val | Ala | Leu | Gly | Asn |
| | | 915 | | | | | 920 | | | | | 925 | | | |
| Met | Tyr | Glu | Leu | Lys | His | Ala | Ser | His | Ile | Ser | Lys | Leu | Pro | Lys | Gly |
| | 930 | | | | | 935 | | | | | 940 | | | | |
| Lys | His | Ser | Val | Lys | Gly | Leu | Gly | Lys | Thr | Thr | Pro | Asp | Pro | Ser | Ala |
| 945 | | | | | 950 | | | | | 955 | | | | | 960 |
| Asn | Ile | Ser | Leu | Asp | Gly | Val | Asp | Val | Pro | Leu | Gly | Thr | Gly | Ile | Ser |
| | | | | 965 | | | | | 970 | | | | | 975 | |
| Ser | Gly | Val | Asn | Asp | Thr | Ser | Leu | Leu | Tyr | Asn | Glu | Tyr | Ile | Val | Tyr |
| | | | 980 | | | | | 985 | | | | | 990 | | |
| Asp | Ile | Ala | Gln | Val | Asn | Leu | Lys | Tyr | Leu | Leu | Lys | Leu | Lys | Phe | Asn |
| | | 995 | | | | | 1000 | | | | | 1005 | | | |
| Phe | Lys | Thr | Ser | Leu | Trp | | | | | | | | | | |
| | 1010 | | | | | | | | | | | | | | |

Isolation, expression, and characterization of human poly(ADP-ribose) polymerase is described in Suzuki et al., Biochem. Biophys. Res. Commun. 146(2):403-409 (1987); Uchida et al., Biochem. Biophys. Res. Commun. 148(2):617-622 (1987); Schneider et al., Eur. J. Cell Biol. 44(2):302-307 (1987); Kurosaki et al., J. Biol. Chem. 262(33):15990-15997 (1987); Cherney et al., Proc. Natl. Acad. Sci. USA 84(23):8370-8374 (1987); Auer et al., DNA 8(8):575-580 (1989); Ogura et al., Biochem. Biophys. Res. Commun. 167 (2):701-710 (1990); Gradwohl et al., Proc. Natl. Acad. Sci. USA 87(8):2990-2994 (1990); Yokoyama et al., Eur. J. Biochem. 194(2):521-526 (1990); NCBI Accession No. A29725, which are hereby incorporated by reference.

Human poly(ADP-ribose) polymerase is encoded by a DNA molecule having a nucleotide sequence according to SEQ. ID. No. 21 as follows:

| | | | | | | |
|---|---|---|---|---|---|---|
| aatctatcag | ggaacggcgg | tggccggtgc | ggcgtgttcg | gtgcgctctg | gccgctcagg | 60 |
| ccgtgcggct | gggtgagcgc | acgcgaggcg | gcgaggcggc | aagcgtgttt | ctaggtcgtg | 120 |
| gcgtcgggct | tccggagctt | tggcggcagc | taggggagga | tggcggagtc | ttcggataag | 180 |
| ctctatcgag | tcgagtacgc | caagagcggg | cgcgcctctt | gcaagaaatg | cagcgagagc | 240 |
| atccccaagg | actcgctccg | gatggccatc | atggtgcagt | cgcccatgtt | tgatggaaaa | 300 |
| gtcccacact | ggtaccactt | ctcctgcttc | tggaaggtgg | gccactccat | ccggcaccct | 360 |
| gacgttgagg | tggatgggtt | ctctgagctt | cggtgggatg | accagcagaa | agtcaagaag | 420 |
| acagcggaag | ctggaggagt | gacaggcaaa | ggccaggatg | gaattggtag | caaggcagag | 480 |
| aagactctgg | gtgactttgc | agcagagtat | gccaagtcca | acagaagtac | gtgcaagggg | 540 |
| tgtatggaga | agatagaaaa | gggccaggtg | cgcctgtcca | agaagatggt | ggacccggag | 600 |
| aagccacagc | taggcatgat | tgaccgctgg | taccatccag | gctgctttgt | caagaacagg | 660 |
| gaggagctgg | gtttccggcc | cgagtacagt | gcgagtcagc | tcaagggctt | cagcctcctt | 720 |
| gctacagagg | ataaagaagc | cctgaagaag | cagctcccag | gagtcaagag | tgaaggaaag | 780 |
| agaaaaggcg | atgaggtgga | tggagtggac | gaagtggcga | agaagaaatc | taaaaaagaa | 840 |
| aaagacaagg | atagtaagct | tgaaaaagcc | ctaaaggctc | agaacgacct | gatctggaac | 900 |
| atcaaggacg | agctaaagaa | agtgtgttca | actaatgacc | tgaaggagct | actcatcttc | 960 |
| aacaagcagc | aagtgccttc | tggggagtcg | gcgatcttgg | accgagtagc | tgatggcatg | 1020 |
| gtgttcggtg | ccctccttcc | ctgcgaggaa | tgctcgggtc | agctggtctt | caagagcgat | 1080 |
| gcctattact | gcactgggga | cgtcactgcc | tggaccaagt | gtatggtcaa | gacacagaca | 1140 |
| cccaaccgga | aggagtgggt | aaccccaaag | gaattccgag | aaatctctta | cctcaagaaa | 1200 |
| ttgaaggtta | aaaagcagga | ccgtatattc | cccccagaaa | ccagcgcctc | cgtggcggcc | 1260 |
| acgcctccgc | cctccacagc | ctcggctcct | gctgctgtga | actcctctgc | ttcagcagat | 1320 |
| aagccattat | ccaacatgaa | gatcctgact | ctcgggaagc | tgtcccggaa | caaggatgaa | 1380 |
| gtgaaggcca | tgattgagaa | actcgggggg | aagttgacgg | ggacggccaa | caaggcttcc | 1440 |
| ctgtgcatca | gcaccaaaaa | ggaggtggaa | aagatgaata | agaagatgga | ggaagtaaag | 1500 |
| gaagccaaca | tccgagttgt | gtctgaggac | ttcctccagg | acgtctccgc | ctccaccaag | 1560 |
| agccttcagg | agttgttctt | agcgcacatc | ttgtcccctt | ggggggcaga | ggtgaaggca | 1620 |
| gagcctgttg | aagttgtggc | cccaagaggg | aagtcagggg | ctgcgctctc | caaaaaaagc | 1680 |
| aagggccagg | tcaaggagga | aggtatcaac | aaatctgaaa | agagaatgaa | attaactctt | 1740 |
| aaaggaggag | cagctgtgga | tcctgattct | ggactggaac | actctgcgca | tgtcctggag | 1800 |
| aaaggtggga | aggtcttcag | tgccaccctt | ggcctggtgg | acatcgttaa | aggaaccaac | 1860 |
| tcctactaca | agctgcagct | tctggaggac | gacaaggaaa | acaggtattg | gatattcagg | 1920 |
| tcctggggcc | gtgtgggtac | ggtgatcggt | agcaacaaac | tggaacagat | gccgtccaag | 1980 |
| gaggatgcca | ttgagcagtt | catgaaatta | tatgaagaaa | aaaccgggaa | cgcttggcac | 2040 |
| tccaaaaatt | tcacgaagta | tcccaaaaag | ttttaccccc | tggagattga | ctatggccag | 2100 |
| gatgaagagg | cagtgaagaa | gctcacagta | aatcctggca | ccaagtccaa | gctccccaag | 2160 |
| ccagttcagg | acctcatcaa | gatgatcttt | gatgtggaaa | gtatgaagaa | agccatggtg | 2220 |
| gagtatgaga | tcgaccttca | gaagatgccc | ttggggaagc | tgagcaaaag | gcagatccag | 2280 |
| gccgcatact | ccatcctcag | tgaggtccag | caggcggtgt | ctcagggcag | cagcgactct | 2340 |
| cagatcctgg | atctctcaaa | tcgcttttac | accctgatcc | cccacgactt | tgggatgaag | 2400 |
| aagcctccgc | tcctgaacaa | tgcagacagt | gtgcaggcca | aggtggaaat | gcttgacaac | 2460 |
| ctgctggaca | tcgaggtggc | ctacagtctg | ctcaggggag | ggtctgatga | tagcagcaag | 2520 |
| gatcccatcg | atgtcaacta | tgagaagctc | aaaactgaca | ttaaggtggt | tgacagagat | 2580 |
| tctgaagaag | ccgagatcat | caggaagtat | gttaagaaca | ctcatgcaac | cacacacagt | 2640 |
| gcgtatgact | tggaagtcat | cgatatcttt | aagatagagc | gtgaaggcga | atgccagcgt | 2700 |
| tacaagccct | ttaagcagct | tcataaccga | agattgctgt | ggcacgggtc | caggaccacc | 2760 |
| aactttgctg | ggatcctgtc | ccagggtctt | cggatagccc | cgcctgaagc | gcccgtgaca | 2820 |
| ggctacatgt | ttggtaaagg | gatctatttc | gctgacatgg | tctccaagag | tgccaactac | 2880 |
| taccatacgt | ctcagggaga | cccaataggc | ttaatcctgt | tgggagaagt | tgcccttgga | 2940 |
| aacatgtatg | aactgaagca | cgcttcacat | atcagcaggt | tacccaaggg | caagcacagt | 3000 |
| gtcaaaggtt | tgggcaaaac | tacccctgat | ccttcagcta | acattagtct | ggatggtgta | 3060 |
| gacgttcctc | ttgggaccgg | gatttcatct | ggtgtgatag | acacctctct | actatataac | 3120 |
| gagtacattg | tctatgatat | tgctcaggta | aatctgaagt | atctgctgaa | actgaaattc | 3180 |
| aattttaaga | cctccctgtg | gtaattggga | gaggtagccg | agtcacaccc | ggtggctgtg | 3240 |
| gtatgaattc | acccgaagcg | cttctgcacc | aactcacctg | gccgctaagt | tgctgatggg | 3300 |
| tagtacctgt | actaaaccac | ctcagaaagg | attttacaga | aacgtgttaa | aggttttctc | 3360 |
| taacttctca | agtcccttgt | tttgtgttgt | gtctgtgggg | aggggttgtt | ttggggttgt | 3420 |
| ttttgttttt | tcttgccagg | tagataaaac | tgacatagag | aaaaggctgg | agagagattc | 3480 |
| tgttgcatag | actagtccta | tggaaaaaac | caaagcttcg | ttagaatgtc | tgccttactg | 3540 |
| gtttccccag | ggaaggaaaa | atacacttcc | accctttttt | ctaagtgttc | gtctttagtt | 3600 |
| ttgattttgg | aaagatgtta | agcatttatt | tttagttaaa | ataaaaacta | atttcatact | 3660 |

The coding sequence is nt 160-3204 (see Cherney et al., Proc. Natl. Acad. Sci. USA 84(23):8370-8374 (1987); NCBI Accession No. M32721 (1995), which are hereby incorporated by reference). Using the DNA molecule for poly(ADP-ribose) polymerase, the DNA molecule can be ligated into an appropriate expression vector, either in sense or antisense orientation, for subsequent transformation of host cells and expression of either poly(ADP-ribose) polymerase or antisense RNA.

For therapeutic purposes, the treated cell is preferably in vivo and the protein or polypeptide or RNA molecule is delivered into the cell in a manner which affords the protein or polypeptide or RNA molecule to be active within the cell. A number of known delivery techniques can be utilized for the delivery, into cells, of either proteins or polypeptides or RNA, or DNA molecules encoding them.

Regardless of the particular method of the present invention which is practiced, when it is desirable to contact a cell (i.e., to be treated) with a protein or polypeptide or RNA molecule, it is preferred that the contacting be carried out by delivery of the protein or polypeptide or RNA molecule into the cell.

One approach for delivering protein or polypeptides or RNA molecules into cells involves the use of liposomes. Basically, this involves providing a liposome which includes that protein or polypeptide or RNA to be delivered, and then contacting the target cell with the liposome under conditions effective for delivery of the protein or polypeptide or RNA into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference.

An alternative approach for delivery of proteins or polypeptides involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference.

Yet another approach for delivery of proteins or polypeptides involves preparation of chimeric proteins according to U.S. Pat. No. 5,817,789 to Heartlein et al., which is hereby incorporated by reference. The chimeric protein can include a ligand domain and, e.g., BVR or a fragment or variant thereof. The ligand domain is specific for receptors located on a target cell. Thus, when the chimeric protein is delivered intravenously or otherwise introduced into blood or lymph, the chimeric protein will adsorb to the targeted cell, and the targeted cell will internalize the chimeric protein.

When it is desirable to achieve heterologous expression of a desirable protein or polypeptide or RNA molecule in a target cell, DNA molecules encoding the desired protein or polypeptide or RNA can be delivered into the cell. Basically, this includes providing a nucleic acid molecule encoding the protein or polypeptide and then introducing the nucleic acid molecule into the cell under conditions effective to express the protein or polypeptide or RNA in the cell. Preferably, this is achieved by inserting the nucleic acid molecule into an expression vector before it is introduced into the cell.

When transforming mammalian cells for heterologous expression of a protein or polypeptide, an adenovirus vector can be employed. Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, which are hereby incorporated by reference. Adeno-associated viral gene delivery vehicles can be constructed and used to deliver a gene to cells. The use of adeno-associated viral gene delivery vehicles in vitro is described in Chatterjee et al., Science 258:1485-1488 (1992); Walsh et al., Proc. Nat'l. Acad. Sci. 89:7257-7261 (1992); Walsh et al., J. Clin Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), which are hereby incorporated by reference. In vivo use of these vehicles is described in Flotte et al., Proc. Nat'l Acad. Sci. 90:10613-10617 (1993); and Kaplitt et al., Nature Genet. 8:148-153 (1994), which are hereby incorporated by reference. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, which are hereby incorporated by reference).

Retroviral vectors which have been modified to form infective transformation systems can also be used to deliver nucleic acid encoding a desired protein or polypeptide or RNA product into a target cell. One such type of retroviral vector is disclosed in U.S. Pat. No. 5,849,586 to Kriegler et al., which is hereby incorporated by reference.

Regardless of the type of infective transformation system employed, it should be targeted for delivery of the nucleic acid to a specific cell type. For example, for delivery of the nucleic acid into tumor cells, a high titer of the infective transformation system can be injected directly within the tumor site so as to enhance the likelihood of tumor cell infection. The infected cells will then express the desired protein product, in this case BVR, or fragments or variants thereof, to immolate the cancer cell.

A further aspect of the present invention relates to a method of treating cells following stroke/ischemic event which is carried out by contacting a cell with biliverdin reductase, or fragment or variant thereof, under conditions effective to inhibit cell damage following stroke/ischemic event. By inhibit cell damage, it is intended to prevent cell damage which is sufficient to cause cell death. Cells which can be treated include mammalian cells, preferably but not exclusively, nerve cells, kidney cells, and heart cells. In addition, it is also desirable, as noted above, to inhibit the activity of poly (ADP-ribose) polymerase in cells following stroke/ischemic event. Antisense RNA which is capable of hybridizing to RNA transcripts coding for poly (ADP-ribose) polymerase can be utilized to this end. Other known regulators of poly (ADP-ribose) polymerase can also be employed.

Whether the proteins or polypeptides or nucleic acids are administered alone or in combination with pharmaceutically or physiologically acceptable carriers, excipients, or stabilizers, or in solid or liquid form such as, tablets, capsules, powders, solutions, suspensions, or emulsions, they can be administered orally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. For most therapeutic purposes, the proteins or polypeptides or nucleic acids can be administered intravenously.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the proteins or polypeptides or nucleic acids in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Both the biliverdin reductase, or fragment or variant thereof, and the antisense RNA can be delivered to the target cells (i.e., at or around the site of the stroke/ischemic event) using the above-described methods for delivering such therapeutic products. In delivering the therapeutic products to nerve cells in the brain, consideration should be provided to negotiation of the blood-brain barrier. The blood-brain barrier typically prevents many compounds in the blood stream from entering the tissues and fluids of the brain. Nature provides this mechanism to insure a toxin-free environment for neurologic function. However, it also prevents delivery to the brain of compounds, in this case neuroprotective compounds that can inhibit nerve cell death following an ischemic event.

One approach for negotiating the blood-brain barrier is described in U.S. Pat. No. 5,752,515 to Jolesz et al., which is hereby incorporated by reference. Basically, the blood-brain barrier is temporarily “opened” by targeting a selected location in the brain and applying ultrasound to induce, in the central nervous system (CNS) tissues and/or fluids at that location, a change detectable by imaging. A protein or polypeptide or RNA molecule of the present invention can delivered to the targeted region of the brain while the blood-brain barrier remains “open,” allowing targeted neuronal cells to uptake the delivered protein or polypeptide or RNA. At least a portion of the brain in the vicinity of the selected location can be imaged, e.g., via magnetic resonance imaging, to confirm the location of the change. Alternative approaches for negotiating the blood-brain barrier include chimeric peptides and modified liposome structures which contain a PEG moiety (reviewed in Pardridge, J. Neurochem. 70:1781-1792 (1998), which is hereby incorporated by reference), as well as osmotic opening (i.e., with bradykinin, mannitol, RPM7, etc.) and direct intracerebral infusion (Kroll et al., Neurosurgery 42(5):1083-1100 (1998), which is hereby incorporated by reference).

A further aspect of the present invention relates to an isolated antibody or binding portion thereof raised against a BVR fragment or variant of the present invention and, therefore, capable of binding the same. The antibodies can be monoclonal or polyclonal.

Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature, 256:495 (1975), which is hereby incorporated by reference.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents. (See Milstein and Kohler, Eur. J. Immunol., 6:511 (1976), which is hereby incorporated by reference.) This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the BVR fragment or variant of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized with pentobarbital 150 mg/Kg IV. This and other procedures for raising polyclonal antibodies are disclosed in Harlow et. al., editors, Antibodies: A Laboratory Manual (1988), which is hereby incorporated by reference.

In addition to utilizing whole antibodies, binding portions of such antibodies can be used. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, New York: Academic Press, pp. 98-118 (1983), which is hereby incorporated by reference.

The antibodies or binding portions thereof can serve a number of uses. One such use includes purification and isolation of BVR fragments or variants which have bound to a particular substrate, e.g., a protein kinase or another protein of interest. This can ultimately enable isolation of the substrate. Another use includes immunostaining techniques for in vivo visualizing intracellular localization of BVR fragments or variants which have bound to a particular substrate. Any suitable label can be attached to the antibody of the present invention to enable its use as a marker during intracellular localization studies, including without limitation fluorescent labels, radiolabeled, or other labels known in the art.

### EXAMPLES

The following Examples are intended to be illustrative and in no way are intended to limit the scope of the present invention.

### Example 1—Identification of Structural Requirements for NADH- and NADPH-Specific Activities and Nuclear Translocation

Materials and Methods

Chemicals:

Cofactors and biliverdin-HCl were purchased from Sigma Chemical Co. The sources of other reagents are noted in connection with the appropriate experiments. All chemicals used were of the highest purity commercially available. Oligonucleotides for mutagenesis and sequencing were purchased from Midland Certified Reagent Company. Fish sperm DNA, Sequenase version 2.0, and all restriction enzymes were obtained from US Biochemical. Reagents for protein determination were obtained from BioRad. Agarose was supplied by GIBCO BRL, bacterial growth media was purchased from Difco, and [α-³⁵S]dATP [S] was purchased from Amersham. The purified rat liver reductase, which was judged to be homogenous by SDS-PAGE stained with Coomasie Brilliant Blue (Sigma), was used for preparation of polyclonal antibody in New Zealand rabbits (Kutty et al., J. Biol. Chem. 256:3956-3962 (1981), which is hereby incorporated by reference).

Purification of E. coli Expressed Human BVR and Measurement of Activity:

To generate glutathione-s-transferase (“GST”)-BVR fusion proteins, wild type hBVR (McCoubrey et al., Eur. J Biochem. 222:597-603 (1994), which is hereby incorporated by reference), plasmids and various mutant HBVR constituents produced for this study were used as templates for PCR reaction using the following primers:

Forward primer, representing nt −3 to +22 (SEQ. ID. No. 22)

| | | | |
|---|---|---|---|
| GGTCGACGAA | TGCAGAGCCC | GAGAG | 25 |

and the reverse primer, representing the reverse complement of nt+881→991 plus vector sequences (italicized) (SEQ. ID. No. 23)

| | | | |
|---|---|---|---|
| GGGCGAATTC | GTCGACTTAC | TTCCTTG | 27 |

SalI linkers are underlined in each primer. The products were digested with SalI and cloned into the vector pGEX 4T-2. Orientation was determined by restriction analysis and confirmed by sequencing. The ligation places the BVR coding region in frame with the GST protein of the vector. GST-BVR fusion proteins were purified from bacterial clones containing the plasmid, and grown overnight using a glutathione-sepharose 4B column (Pharmacia). The reductase portion of the fusion protein was released by thrombin protease treatment.

Activity Measurement and Western Blot Analysis:

Activity was determined as previously described (Kutty et al., J. Biol. Chem. 256:3956-3962 (1981); Huang et al., J. Biol. Chem. 264:7844-7849 (1989), which are hereby incorporated by reference). Activity was determined in a 1 ml assay volume that contained 0.1 M Tris/HCl, pH 8.7 NADPH (100 μM), and biliverdin (5 μM). Reductase activity was also measured using NADH (1 mM) as the cofactor; in this case 0.1 M potassium phosphate pH 6.75, was substituted for Tris/HCl. SDS/PAGE was performed by the method of Laemmli, Nature 227:680-685 (1970), which is hereby incorporated by reference. Western blot analysis was performed using anti-serum to human kidney biliverdin reductase as previously described (Maines et al., Arch. Biochem. Biophys. 300:320-326 (1993), which is hereby incorporated by reference). The relative amounts of immunoreactive protein were determined by scanning the blot using a laser densitometer and comparing the area under the peak for each mutant with the wild type. The rate of activity was measured as the increase in 450 nm absorbance at 25° C. Specific activity is expressed as nmol bilirubin/min/ mg protein.

Site Directed Mutagenesis of Human BVR:

The expression clone hBVR-1 (Maines et al., Eur. J. Biochem. 235:372 (1996), which is hereby incorporated by reference) was used to generate all mutants used in these studies. Mutant constructs included: Gly¹⁷, Ser⁴⁴, Lys⁹²/His⁹³, Ser¹⁴⁹, Ser ¹⁵³/Thr¹⁵⁴, Cys⁷⁴, Cys⁷⁴+Cys²⁰⁴, Cys²⁸¹, Cys²⁹², Cys²⁹²+CyS²⁹³ and all 5 Cys residues combined, in the GST expression systems. Also a construct consisting of the carboxy terminal aa²⁷²⁻²⁹⁶ deleted protein was generated. Site directed or PCR methods were employed to introduce mutations; the method that was used depended on the mutation site(s) within the BVR coding region. For mutations near the carboxyl terminus, such as Cys²⁹² and Cys²⁹³, a PCR mediated mutagenesis was used. In this method, the mutation(s) was introduced in a reverse primer used to amplify the full-length cDNA by PCR as described in purification of cloned reductase. The primers also include enzyme linkers (SalI), which was used for cloning into the appropriate vector. Other mutations were introduced as before (McCoubrey et al., Eur. J Biochem. 222:597-603 (1994); Maines et al., Eur. J. Biochem. 235:372 (1996), which are hereby incorporated by reference) by the oligonucleotide-mediated single stranded method. All mutations change the target amino acid(s) to an alanine residue (GCN codon). Substitution with alanine is the most commonly used in site-directed mutagenesis. E. coli mut S cells were transformed and miniprep DNA was prepared from this mixed culture (containing wild type or mutated DNA) and used to transform E. coli InvαF′ cells. The mutants were identified by sequence analysis. Multiple mutations were generated by using single-stranded DNA from single-stranded DNA from single mutants for additional rounds of mutagenesis. Oligo-nucleotide primers used for mutagenesis of cloned human BVR were based on hBVR of SEQ. ID. No. 1.

Circular Dichroism Analysis:

Affinity-purified homogenous as established by BVR from strains expressing wild-type or mutant (Cys²⁸¹→Ala) protein was used. Samples in 20 mM potassium phosphate, pH 7.2, were analyzed in a 0.2 cm cell in a Jasco J600 Spectropolarimeter at 20° C. Scans were collected at 10 nm/min with a time constant of 1 s using 50 μg/ml of protein and are the average of two scans. Data were smoothed using spline fitting.

Phosphotransferase Activity:

The “in solution” kinase assay protocol of Brown et al., Mol. Cell Biol. 9:1803-1816 (1998), which is hereby incorporated by reference) was used at pH 6.7, 7.4, and 8.7. The reaction mix contained 30 mM Tris-HCl at the appropriate pH, 0.5 mM DTT, 30 μM ATP (Sigma), and 5 μCi [γ³²P]-ATP/50 pl. Metal dependence of BVR kinase activity was measured in the presence of 0, 1, 2 mM Ca²⁺ and/or 0, 1, 2 mM Mn²⁺. The reaction contained 5 μg purified BVR or 10 μg dephosphorylated α-casein (Sigma), or both. The assay mix was incubated at 37° C. for 1 h and was terminated by adding 10 μl 1% SDS. Excess ATP was removed by gel filtration through a Sephadex G-50 column (Pharmacia) and the eluant was loaded onto a 0.75 mm, 15% SDS-PAGE. The gel was stained using Coomassie blue and dried under a vacuum. The same gel was then exposed to Hyperfilm ECL (Amersham) and developed.

Results and Discussion

Motif search was used to identify residues of potential significance to phosphorylation. The mutations were introduced by changing those residues to Ala and included:
a) Gly¹⁷ of Gly¹⁵.Xaa.Gly.Xaa.Xaa.Gly²⁰ motif, which is found in various adenine nucleotide-binding proteins,
b) Ser⁴⁴ in Ser.Lys.Arg motif;
c) Ser¹⁵⁴ in Phe.Thr.Ser motif;
d) Ser¹⁴⁹ in Lys.Gly.Ser motif; and
e) Lys⁹²/His⁹³ of the “oxidoreductase” motif Ala.Gly.Lys.His.Val were substituted.

All these constructs were expressed, purified and evaluated for its importance to BVR activity. The NADH- and NADPH-specific activities of several of these mutants are shown in Table 1 below.

TABLE 1
Effect of BVR Mutations on Reductase
Activity with NADH or NADPH

Activity (nmol bilirubin/min/mg)

| Site of Mutation | pH 6.7 + NADH | pH 8.7 + NADPH |
|---|---|---|
| wild type | 2830 | 1338 |
| Gly¹⁷ | 698 | 0 |
| Ser⁴⁴ | 7443 | 333 |
| Ser¹⁴⁹ | 0 | 231 |
| Lys⁹²/His⁹³ | 0 | 0 |
| PGE2 | 0 | 0 |

The rate of enzyme activity was measured at 25° at pH 6.7 with NADH or at pH 8.7 with NADPH as the cofactor. PGE2 is the vector only.

The Gly¹⁷ mutant preparation was inactive at pH 8.7 with NADPH and possessed moderate activity (˜20-25% of the control) with NADH at pH 6.7. The Ser¹⁴⁹ mutant preparation was inactive at pH 6.7 with NADH and possessed minimal activity at pH 8.7 with NADPH. Ser¹⁵⁴ mutation did not substantially effect enzyme activity at pH 6.7 and moderately decreased activity at pH 8.7 to about 71% of the control.

Interestingly, the construct with mutation in Ser⁴⁴ showed a remarkable 2.5-fold increase in activity at pH 6.7 with NADH, and a pronounced reduction (70-75%) in NADPH-dependent activity. It appears that phosphate interactive residues are more important to differential pH/cofactor preference of the enzyme than the “oxidoreductase” domain or the cysteine residues at the carboxy terminus of the reductase that are suspected to be involved in Zn-binding (Maines et al., Eur. J. Biochem. 235:372 (1996), which is hereby incorporated by reference). Also, the Lys⁹²/His⁹³ to Ala/Ala mutation caused loss of enzyme activity at both pH optima, suggesting that the residues in the domain that is conserved in oxidoreductases is essential to the activity of BVR under both pH/cofactor conditions.

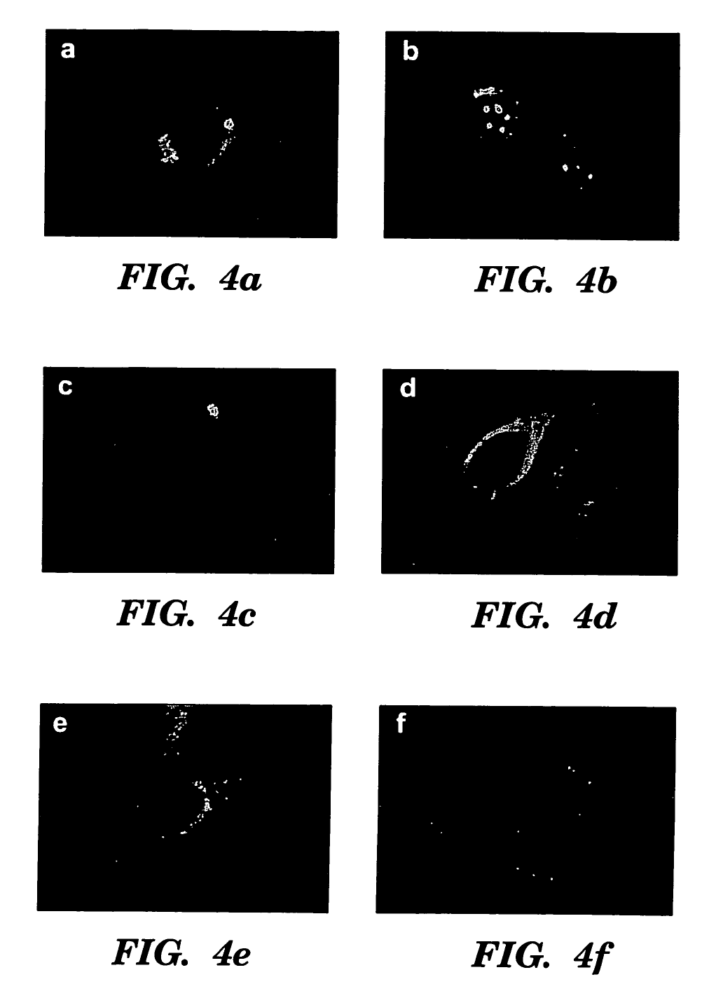

Because of the putative nuclear translocation signal at aa 222 to 227, modification of this domain or the C-terminal 94 aa residues were incorporated into mutant. BVR proteins or polypeptides by expressing hemagglutinin tagged mutant proteins in HeLa cells. After treatment of the cells with 8-bromo-cGMP, cells were examined by fluorescence immunochemistry. FIG. 4A-B illustrate that cGMP is required for nuclear translocation of wild type BVR, whereas mutant BVR, whether treated with cGMP or not, was incapable of nuclear translocation (FIGS. 4C-4F).

As noted in Table 2 below, mutation of the cysteine residues had essentially the same effect on both NADH and NADPH-dependent activities at pH 6.7 and pH 8.7, respectively. Cys⁷⁴ and Cys²⁸¹ are clearly important to enzyme activity. Cys²⁸¹ is found in the His.Cys²⁸¹.Xaa₁₀.Cys.Cys motif of hBVR. Cys²⁰⁴, Cys²⁹² and Cys²⁹³ do not appear of importance to activity.

TABLE 2
Effect of Cysteine Residue Mutation on
Human Biliverdin Reductase Activity

Activity %

| Site of Mutation | pH 6.7 + NADH | pH 8.7 + NADPH |
|---|---|---|
| wild type | 100 | 100 |
| Cys⁷⁴ | 44 | 45 |
| Cys²⁰⁴ | 96 | 94 |
| Cys²⁸¹ | 31 | 23 |
| Cys²⁹²^{,}²⁹³ | 94 | 100 |
| Cys^{74,204}^{,}^{281,292,293} | 1 | 10 |
| Truncated-272-296 | 0 | 0 |

hBVR constructs with the above indicated Cys→Ala mutations were generated and expressed in E. coli. The expressed proteins were purified and analyzed for the rate of enzyme activity to reduce biliverdin to bilirubin.

Data suggest that certain residues in conserved phosphate binding motifs are of significance to display of the unique character of the enzyme. Residues, such as Cys⁷⁴, Cys²⁸¹ and Lys⁹²/His⁹³ in the “oxidoreductase” domain, are required for activity at both pH/cofactor settings, and their absence effects activity at both experimental settings to the same extent. The “oxidoreductase” motif (SEQ. ID. No. 8) is found conserved among oxidoreductases, including both the procaryotic and eukaryotic species. Thus, these data should be broadly applicable to other BVR.

Other residues, however, have differential importance to activity at pH 6.7 with NADH or pH 8.7 with NADH. The residues that were identified in the present study are all in phosphate interacting domains. For instance, mutation of the cysteine residues or the “oxidoreductase” domain effects both activities to the same extent, while mutation of the Gly¹⁷, Ser⁴⁴, and Ser¹⁴⁹ residues has disparate effects. The Gly¹⁵.Xaa.Gly.Xaa.Xaa.Gly²⁰ (SEQ. ID. No. 7) consensus motif in the N terminus of BVR is a conserved motif found in the cyclic nucleotide regulated/binding proteins and protein kinase family (Yarden et al., Annu. Rev. Biochem. 57:443-478 (1988); Schlessinger, Trend. Biochem. Sci. 13:443-447 (1988); Hanks et al., Science 241:42-52 (1988), which are hereby incorporated by reference). This motif is obviously essential to binding of NADPH phosphate, and is indispensable for NADPH-dependent activity. Ser¹⁴⁹ of the Lys.Gly.Ser motif, upstream of the Phe.Thr.Ser motif found in many phospho-binding proteins, is identified as critical to NADH dependent activity. Without being bound by theory, it is believed that the basic residue N terminal to serine phosphorylation site may act as an important substrate determinant (Kemp et al., Proc. Natl. Acad. Sci. USA 80:7471-7475 (1983), which is hereby incorporated by reference). A serine residue has been identified as the contact point for adenine nucleotide in human inosine monophosphate dehydrogenase type II by forming a hydrogen bond with phosphate oxygen (Colby et al., Proc. Natl. Acad. Sci. USA 96:3531-3536 (1999), which is hereby incorporated by reference). Therefore, it is reasonable to suspect that mutation of Ser¹⁴⁹ of BVR impedes nucleotide binding.

Perhaps the most intriguing observation concerns data involving mutation of Ser⁴⁴ in the Ser.Arg.Arg motif (SEQ. ID. No. 10), which caused a remarkable increase in NADH-dependent activity. It may be reasoned that by changing Ser, an uncharged polar residue, to alanine, a hydrophobic residue, the hydrophobic forces that contribute to the overall shape of BVR, are disrupted. The presence of Ala⁴⁴ flanked upstream by hydrophobic residues, Phe⁴⁶ and Val⁴⁵, could effect the free energy of BVR in aqueous medium. In comparison to the wild type, Ser⁴⁴ is upstream of three of polar/charged residues—Arg.Arg.Glu.

The findings that the Gly¹⁷ and Ser¹⁴⁹ mutants displayed reductase activity at pH 6.7 with NADH and 8.7 with NADPH, respectively, albeit a fraction (20-25%) of the wild type activity under the same condition, indicate that the Gly.Xaa.Gly.Xaa.Xaa.Gly (SEQ. ID. No. 7) nucleotide-binding domain is indispensable for binding of NADPH phosphate, and Ser¹⁴⁹ is indispensable for NADH binding.

### Example 2—Phosphorylation of hBVR

Materials and Methods

Chemicals:

Oligonucleotides for mutagenesis and sequencing were purchased from Gibco BRL. Sequenase® versions 2.0, as well as all restriction enzymes were obtained from US Biochemical (Cleveland, Ohio). Reagents for protein determination were obtained from BioRad (Richmond, Calif.). Reagents for cell cultures were purchased GIBCO BRL (Gaithersburg, Md.) or from Difco (Detroit, Mich.). [α³⁵s]-dATP and [γ³²P]-ATP were from New England Nuclear. Reagents for immunostaining were from Vector (Burlingame, Calif.). Antibodies to phospho amino acids were obtained from Zymed Laboratories. Antibody to human BVR was prepared as before (Kutty et al., J. Biol. Chem. 256:3956-3962 (1981), which is hereby incorporated by reference). The experiments conducted in this study were repeated several times and representative data are presented.

Site-directed Mutagenesis of Expressed Human Biliverdin Reductase:

The expression clone hBVR-1 (McCoubrey et al., Eur. J Biochem. 222:597-603 (1994), which is hereby incorporated by reference) was used to generate alanine mutants of Gly¹⁷, Ser¹⁴⁹, Lys²⁹⁶ as well Ser⁴⁴ mutants, using the previously described method (McCoubrey et al., Eur. J Biochem. 222:597-603 (1994), which is hereby incorporated by reference). Using oligonucleotide corresponding to reverse complement of the appropriate BVR cDNA sequence with a mismatch for the first nucleotide of the amino acid of interest.

An additional mutant was generated by the same method and designated “NLS” mutant using the oligonucleotide primer as follows (SEQ. ID. No.24):

| | | | | | |
|---|---|---|---|---|---|
| GGAAGCTTAA | ATATCCTGTG | GATCCTATAAC | AGGTCCTTTT | TC | 42 |

This primer included the reverse complement of nucleotides +652 to 694 with mismatches (underlined) resulting in the change of amino acids 222-227 of SEQ. ID. No.1 from GLKRNR to VIGSTG (SEQ. ID. No.25). The changes made were conservative ones except that charged residues were replaced with uncharged ones. The mutations introduced a BamHI site, the presence of which was used in screening for mutants and the sequence of those mutants was subsequently confirmed.
Purification of Reductase and Measurement of Activity:

GST-BVR fusion proteins were prepared as described in Example 1. Human kidney biliverdin reductase was purified to homogeneity as described by Maines et al., Arch. Biochem. Biophys. 300:320-326 (1993), which is hereby incorporated by reference. The purified reductase preparations were judged to be homogenous by SDS-PAGE. Protein concentration was determined by the method of Bradford, Anal. Biochem. 72:248-254 (1976), which is hereby incorporated by reference.

Activity was determined as previously described by Huang et al., J. Biol. Chem. 264:7844-7849 (1989), which is hereby incorporated by reference, using NADPH as the cofactor at pH 8.7 in 0.1 M potassium phosphate buffer or NADH at pH 6.5 in Tris-HCl. The rate of activity was measured as the increase in 450 nm absorbance at 25° C. Specific activity is expressed in units/mg protein, where 1 unit catalyzes the formation of 1 nmol bilirubin/min.

Generation of Hemagglutinin-tagged Constructs for Transfection:

DNA from plasmids encoding wild type or NLS mutant BVR was used as substrate for PCR using the following primers:

| | |
|---|---|
| GGGATCCATG TACCCCTACG | 55 |
| ACGTGCCCGA CTACGCCAAT | |
| GCAGAGCCCG AGAGGA | |

(SEQ. ID. No. 26) which represents nucleotides+4 to +22 placed, in-frame, downstream of a hemagglutinin recognition sequence (bold), a methionine start codon (underlined) and a BamHI linker; and

| | | | |
|---|---|---|---|
| GCTCGAGCTC | CTCCTCTTAC | TTCCTTG | 27 |

(SEQ. ID. No. 27) which is the reverse complement of nucleotides +881 to +900 including the stop codon (underlined) and an XhoI linker (italics). The product was cloned into the vector pCR2.1 and the insert was excised using BamHI and XhoI and was subcloned into pcDNA3, which had been cut with the same enzymes.

An additional construct consisting of only the C-terminus of the reductase and the hemagglutinin tag was generated using the forward primer as follows:

| | |
|---|---|
| GCTCGAGATG TACCCCTACG | 57 |
| ACGTGCCCGA CTACGCCATG | |
| ACAGTGTGTC TGGAGAC | |

(SEQ. ID. No. 28)) which includes nucleotides +601 to 620, a tag sequence, a start codon, and XhoI linker as noted above. The reverse primer (SEQ. ID. No. 23) was the same one used to generate the full-length constructs. When subcloned into pcDNA3, this construct encoded a tagged protein representing amino acids 200 to 296 of biliverdin reductase.
Autophosphorylation of Biliverdin Reductase:

The ability of the reductase to phosphorylate itself was examined using the in blot method of Ferrell and Martin (Ferrell et al., Methods Enzymol. 200:430-435 (1991), which is hereby incorporated by reference). Purified wild type or mutant proteins were used.

Assessment of the Phosphorylation State of Biliverdin Reductase:

Purified protein was subjected to 12.5% SDS-PAGE and the protein transferred to PVDF membrane. The membrane was subsequently treated as for Western blotting, using anti-phosphoserine, anti-phosphotyrosine or anti-phosphothreonine, at 2 μg/ml, or a mixture of all three antibodies as the primary antibody. The secondary antibody was goat anti-rabbit horseradish peroxidase at a 1:1000 dilution and detection was carried out using the ECL reagent kit (NEN Life Sciences) according to the manufacturer's instructions.

Cell Culture and Immunofluorescence Staining:

HeLa cells were grown on glass cover slips in DMEM medium containing 10% FBS under an atmosphere of 5% CO₂. Cells were transfected with different BVR constructs using Lipofectamine, as described by the manufacturer. Briefly, cells were treated with a mixture of 2 μg DNA and 20 μl Lipofectamine for 5 h followed by the addition of DMEM medium. After 48 h, cells were treated with 500 μM 8-bromo-cGMP for 5, 20 or 60 min, fixed with 4% paraformaldehyde for 10 min. Cells were then rinsed with phosphate-buffered saline, incubated with 5% goat serum to block nonspecific binding sites and then with monoclonal anti hemagglutinin primary antibody and FITC conjugated secondary antibody. Cells were visualized by direct fluorescence microscopy.

Results and Discussion

A small fraction of phosphorylated proteins are autophosphorylated (Hunter et al., Ann. Rev. Biochem. 54:897-930 (1985); Walaas et al., Pharmacol. Rev. 43:299-349 (1991), which are hereby incorporated by reference). The most frequently occurring phosphate esters of amino acids are those of serine and threonine. In comparison, phosphate esterfied to tyrosine is rather rare (less than 1% of autophosphorylated proteins) and is more recently described (Eckhart et al., Cell 18:925-933 (1979), which is hereby incorporated by reference). A common feature of nucleotide binding and autophosphorylated proteins is the conserved motif, Gly.Xaa.Gly.Xaa.Xaa.Gly (SEQ. ID. No. 7), which serves in ATP pyrophosphate binding. The presence of the consensus motif in its predicted primary structure of both rat and human BVR (Fakhrai et al., J. Biol. Chem. 267:4023-4029 (1992); Maines et al., Eur. J. Biochem. 235:372-381 (1996), which are hereby incorporated by reference) prompted this investigation as to its significance to BVR posttranslational modification, catalytic activity, and the potential involvement in phosphotransferase activity.

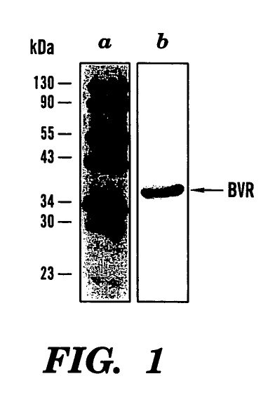

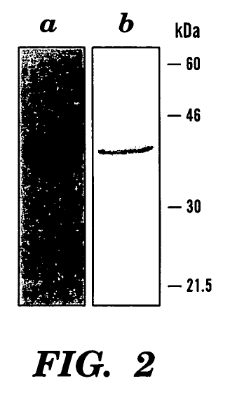

To address whether the hBVR is a phosphoprotein, the protein purified from human kidney was analyzed by Western blotting, utilizing a mixture of antibodies that recognize phosphotyrosine, phosphothreonine, and phosphoserine (called “anti-phospho mix”) as the primary antibody. As shown in FIG. 1 (panel b), when probed with the mix of antibodies, a band corresponding to BVR's molecular size is detected, indicating that human BVR is a phosphoprotein. FIG. 1 (panel a) shows phosphorylation molecular weight markers probed with the same mix of antibodies. Next it was determined whether phosphorylation of BVR is the result of an autokinase activity. As noted in FIG. 2 (panel b), BVR bound to PVDF membrane after denaturation with guanidine-HCl and renaturation, in the presence of [γ³²P]-ATP, produces a single band with a mobility corresponding to that of human BVR, as detected by SDS-gel electrophoresis (FIG. 2, panel a).

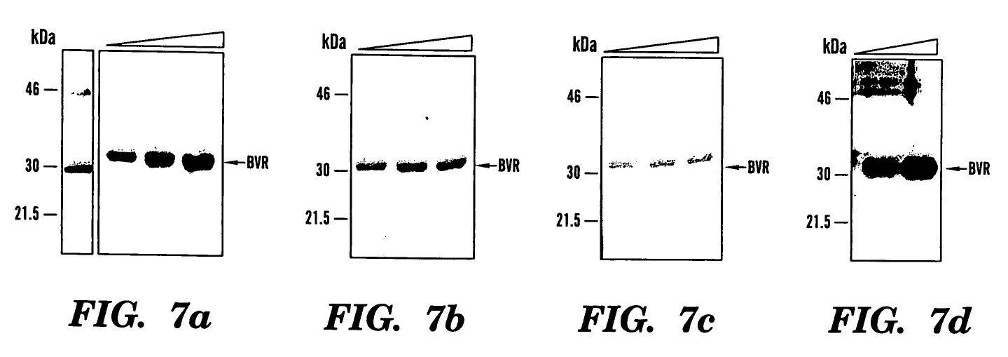

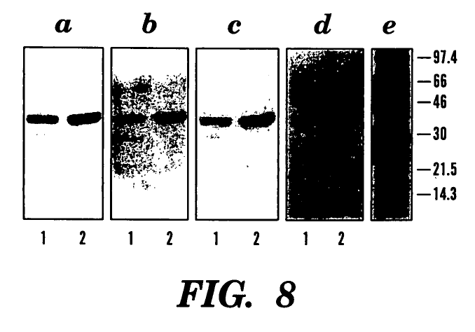

Having established that BVR is a renaturable phosphoprotein, the phosphorylation of serine, threonine, and tyrosine residues individually (FIG. 7) and whether the Gly.Xaa.Gly.Xaa.Xaa.Gly (SEQ. ID. No. 7) consensus is involved in BVR autophosphorylation were examined (FIG. 8). For this, site-directed mutagenesis was employed to change the second glycine, Gly¹⁷, of this motif to alanine and the expressed and purified product of this construct along with that of the wild type E. coli expressed BVR were assessed for autophosphorylation. Data shown in FIG. 8 indicate that E. coli expressed BVR is also phosphorylated on serine, threonine, and tyrosine and that the mutation, which hinders interaction of ATP terminal phosphate with the binding site, decreased the extent of BVR phosphorylation, as indicated by the decreased intensity of the band in lane 1 when compared to lane 2. Consistent with this observation was a reduced level of BVR phosphorylation (FIG. 8, panel a) when the mutant protein (lane 1) and the wild type preparations were probed with anti-phospho-mix. When quantitated by laser densitometry, a reduction in phosphorylation of approximately 50% were detected for the Gly¹⁷ mutant reductase. The magnitude of reduction in phosphorylation was not affected by changes in the assay system. As for the amino acid specificity of BVR phosphotransferase activity, as shown in FIG. 8, phosphotyrosine (panel b) and phosphoserine (panel c) signals, respectively, in the mutant preparation (lane 1) were reduced (˜50%) when compared with that of the wild type preparation (lane 2). A reduction in phosphothreonine (panel d) signal, however, was not evident. The results suggest the Gly.Xaa.Gly.Xaa.Xaa.Gly (SEQ. ID. No. 7) motif of BVR is involved in the transfer of ATP pyrophosphate to serine and tyrosine residues of the protein. Dual phosphorylation of serine as well as tyrosine residues is an uncommon type of phosphotransferase activity.

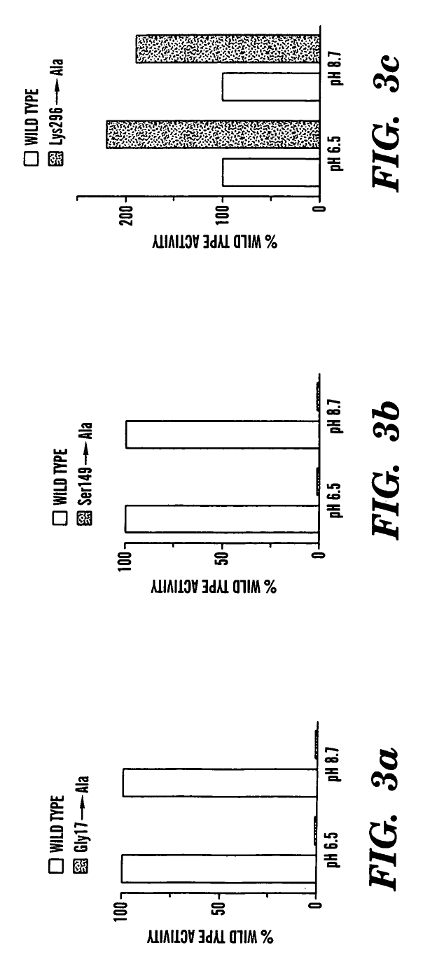

To gain understanding of the significance of autophosphorylation, the following studies were carried out. Because the Gly.Xaa.Gly.Xaa.Xaa.Gly (SEQ. ID. No. 7) motif is found in various adenine nucleotide-binding proteins, the effect of Gly¹⁷ mutation on the catalytic activity of BVR to reduce biliverdin was examined (FIG. 3A). In addition, other constructs were prepared with mutations in residues identified by homology search as of potential importance in phosphotransferase activity and were analyzed for catalytic activity and autophosphorylation. An Ala mutation was introduced at Ser¹⁴⁹ in the Leu¹⁴⁵.Leu.Lys.Gly.Ser.Leu.Leu (within the leucine zipper) sequence flanking the consensus, Phe¹⁵².Thr.Ser motif (SEQ. ID. No. 12), which is found in many kinases. An Ala mutation and in terminal Lys²⁹⁶ was also introduced; this lysine and the lysine at position 290 flank the potential site of zinc binding (Huang et al., J. Biol. Chem. 264:7844-7849 (1989), which is hereby incorporated by reference). The terminal Lys residue can participate in phosphotransfer activity (Kamps et al., Nature 310:589-592 (1984); Zoller et al., J. Biol. Chem. 256:10837-10842 (1981); Russo et al., J. Biol. Chem. 260:5205-5208 (1985), which are hereby incorporated by reference), for example, with oncogene v-src and cyclic GMP dependent kinase, a terminal Lys residue reacts with ATP (Kamps et al., Nature 310:589-592 (1984), which is hereby incorporated by reference). These mutants were also expressed in E. coli and purified. As shown in FIGS. 3A and 3B, respectively, when assayed for BVR catalytic activity, the Gly¹⁷ and Ser¹⁴⁹ mutant preparations were essentially inactive at pH 6.5, in the presence of NADH, and at pH 8.7, using NADPH as cofactor. The finding indicates that the Gly.Xaa.Gly.Xaa.Xaa.Gly (SEQ. ID. No. 7) nucleotide-binding domain is indispensable for catalytic activity. Also, it identifies Ser¹⁴⁹ as indispensable for activity. A serine residue has been recently identified as the contact point for adenine nucleotide in human inosine monophosphate dehydrogenase type II by forming a hydrogen bond with phosphate oxygen (Colby et al., Proc. Natl. Acad. Sci. USA 96:3531-3536 (1999), which is hereby incorporated by reference). Therefore, it is reasonable to suspect that mutation of Ser¹⁴⁹ of BVR impeded cofactor nucleotide binding. Interestingly, the construct with mutation in the terminal lysine (amino acid 296) showed a doubling of activity at both pH optima (FIG. 3C). At this time, the molecular basis for increase in BVR activity in Lys²⁹⁶ mutant protein is not known.

Another mutant BVR protein containing a Ser⁴⁴→Ala mutation in the Ser⁴⁴.Arg.Arg sequence (SEQ. ID. No. 10), which is a conserved domain in phosphotransferases, did not display loss of enzyme activity.

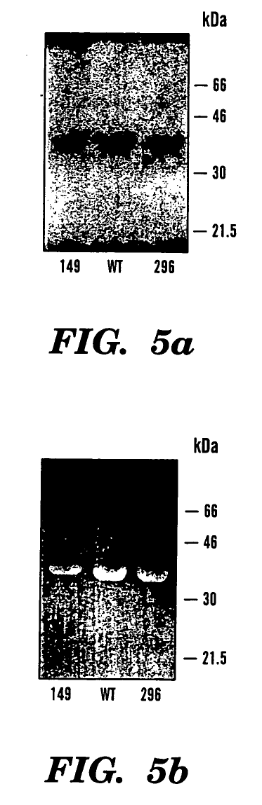

Because the phosphorylation state of many proteins regulates their catalytic function, experiments were performed to determine whether the mutations that alter enzyme activity also effect autophosphorylation of the expressed protein. For this, the Ser¹⁴⁹→Ala and Lys²⁹⁶→Ala mutant expressed BVR mutants were analyzed. As shown in FIG. 5A, autophosphorylation activity of the mutant proteins, when corrected for protein loading (FIG. 5B) was essentially comparable to that of the wild type protein. (A smaller amount of the Ser¹⁴⁹ mutant was loaded because the clone with this mutation did not express well.) Collectively, the findings suggest the possibility that the autophosphorylation and reductase activities of BVR are separate properties of the enzyme.

To continue the effort toward understanding a possible significance of BVR autophosphorylation, the primary features of the protein were considered. The cluster of positively charged residues in the carboxy terminal third of the protein, which has the criteria ascribed to nuclear localization signal (NLS) (Garcia-Bustos et al., Biochim. Biophys. ACTA 1071:83-101 (1991), which is hereby incorporated by reference), was found noteworthy. Such clusters are present in certain phosphotransferases, like those that phosphorylate G-protein-coupled receptors (Hanks et al., Methods Enzymol. 200:38-62 (1991), which is hereby incorporated by reference) and those that translocate in the cell in response to phosphorylation activators, such as cGMP (Newton, Curr. Biol. 5:973-976Z (1995); Mochly-Rosen et al., Adv. Pharmacol. 44:91-145 (1998); Fowler et al., Cell Growth Diff. 9:405-413 (1998), which are hereby incorporated by reference).

BVR has traditionally been considered as a protein exclusive to the cytosol. Accordingly, in HeLa cells, localization of a BVR construct, in which the sequence encompassing the putative NLS, corresponding to amino acids 222-227 of SEQ. ID. No. 1 (GLKRNR), was mutated to VIGSTG (SEQ. ID. No. 25) was examined under normal growth conditions and in response to the cGMP analog, 8-bromo-cGMP. Comparison was made with localization of the wild type under the same conditions. To monitor cellular location, the constructs were designed with a short hemagglutinin tag at the amino terminus. Immunofluorescence staining, using anti-hemagglutinin as the primary antibody, was used to trace the tagged proteins. Results are shown in FIGS. 4A-F. Under normal conditions, wild type BVR was found diffused and distributed throughout the cytoplasm with a somewhat greater concentration in the perinuclear region (FIG. 4A). Five min after treatment with the cGMP analog there was a lessening of cytoplasmic staining and the appearance of strong punctate staining associated with the nucleus (FIG. 4B). The cellular localization of the “NLS” mutant protein, under control conditions, was nearly indistinguishable from what was observed with the wild type protein (compare FIG. 4C versus FIG. 4A). However, as shown in FIG. 4D, nuclear localization of the mutant protein was not detected in response to 8-bromo-cGMP stimulation. Nuclear localization at shorter and longer time points (up to 60 min) were also examined and, again, translocation was not detected. It appears certain that mutation in this region of BVR alters intracellular trafficking of the protein in response to the cyclic nucleotide.

The feature(s) of BVR that are relevant to its nuclear association was further examined. In addition to the putative NLS, in the carboxy terminal region of BVR (amino acids 202-296), is located the Cys/His-rich putative zinc binding motif. Therefore, whether this fragment of BVR is capable of nuclear translocation was examined. Similar experiments as above were conducted with HeLa cells transfected with a construct that expressed this fragment. As noted in FIG. 4E, the truncated protein localized to the cytoplasm, and in response to the cyclic nucleotide (FIG. 4F), it showed only perinuclear association. This finding suggests a requirement for features that are present in the holoprotein that permit BVR nuclear translocation.

The finding that BVR translocates in the cell as the result of stimulation with cGMP raises the intriguing possibility that the enzyme may have a function in intracellular trafficking of regulatory factors or itself has a nuclear function. Noteworthy is our recent observation that rBVR is a PKC activating protein (infra). The fact that BVR is a zinc metalloprotein, and the presence of a leucine zipper motif (Leu¹²⁷-Xaa₆-Leu-Xaa₆-Lys-Xaa₆-Leu-Xaa₆-Leu, SEQ. ID. No. 9) in the protein, may be of relevance to its potential nuclear activity. In BVR, as in GCN4, cMyc, YAP-1, Fos and Jun, a lysine (or arginine) residue substitutes for one of the leucines (Busch et al., Trend Genet. 6:36-40 (1990), which is hereby incorporated by reference).

The existence of a high affinity metalloporphyrin-binding site of BVR, which was distinct from its substrate (biliverdin) binding site, has been previously described (Bell et al., Arch. Biochem. Biophys. 263:1-19 (1988), which is hereby incorporated by reference). Given the fact that metalloporphyrins are effective regulators of gene expression (Granick et al., J. Biol. Chem. 250:9215-9225 (1975); Foresti et al., J. Biol. Chem. 272:18411-18417 (1997), which are hereby incorporated by reference) it would be reasonable to believe that, potentially, BVR serves as an intracellular shuttle mechanism for heme. The significance of its autophosphorylation may relate, in part, to intracellular transport of regulatory factors.

### Example 3—Protein Kinase Activity of Rat BVR

Materials and Methods

Chemicals:

Cofactors and biliverdin-HCl were purchased from Sigma Chemical Co. Male Sprague-Dawley rats were purchased from Harlan Industries. The sources of other reagents are noted below in connection with the appropriate experiments. All chemicals used were of the highest purity commercially available. BVR peptides K²⁷⁴KRIMHC²⁸⁰ (peptide 1, SEQ. ID. No. 18) and Q²⁸⁸KLCHQKK²⁹⁵ (peptide 2, SEQ. ID. No. 19) and PKC inhibitor peptide RKRCLRRL (SEQ. ID. No. 29) were obtained in HPLC purified form from Primm Laboratories (Andover, Mass.).

Purification of Biliverdin Reductase from Rat Tissue and Production of Antibody:

BVR was purified from rat liver to homogeneity (3,000 nmol bilirubin/min/mg protein) as described before (O'Carra et al., J. Biochem. 125:110P (1971), which is hereby incorporated by reference), with modifications (Huang et al., J. Biol. Chem. 264:7844-7849 (1989), which is hereby incorporated by reference). Activity was determined at pH 8.7 with NADPH in Tris-HCl with NADH at pH 6.7 cofactor in potassium phosphate buffer. The substrate concentration was 5 μM. The rate of activity was measured by the increase in 450 nm absorbance at 25° C. The purified reductase, which was judged to be homogenous by SDS-PAGE, stained with Coomasie Brilliant Blue (Sigma), was used for preparation of polyclonal antibody in New Zealand rabbits (O'Carra et al., J. Biochem. 125:110P (1971), which is hereby incorporated by reference). Protein concentration was determined by Bradford's method (Bradford, Anal. Biochem. 72:248-254 (1976), which is hereby incorporated by reference).

Expression and Purification of Recombinant Biliverdin Reductase in E. coli:

A construct expressing rat BVR as a fusion protein with glutathione-S-transferase (GST) was generated as follows: First strand cDNA was prepared from rat liver RNA using the cDNA Cycle kit (Invitrogen) employing oligo dT primer. The single-stranded DNA was then used as a template for the polymerase chain reaction using the following primers:

| | | | |
|---|---|---|---|
| GGTCGACAGA | GACCGAGTTG | GATGCCGAG | 29 |

(SEQ. ID. No. 30) which consists of nucleotides −10 to +12 of rat BVR with a substitution of T for A at position +1 (bold) and a SalI linker (italicized); and

| | | | |
|---|---|---|---|
| GCGGCCGTCG | TCTCTGAATC | TTCCTCTTC | 29 |

(SEQ. ID. No. 31) which represents the reverse complement of nucleotides +887 to +896 and a NotI linger (italicized). The product was cloned into the vector pCR 2.1; the insert was excised with SalI and NotI and subcloned into pGex 4T2 which had been digested with the same enzymes and was transformed into E. coli Inv α F′. Clones were identified by restriction analysis and inserts were confirmed by sequencing. The ligation places the coding region of rBVR in frame with the GST encoded by the vector. Due to the presence of 5′ UTR sequences, the N terminus is extended by 3 amino acids (Glu-Thr-Glu) and the initial methionine (now at amino acid 4) is replaced by serine. This addition to the N terminus was employed because initial attempts to generate an insert starting with amino acid 1, resulted in internal priming of the cDNA which led to deletion of the first 200 nucleotide and a frame shift in the insert relative to the vector. To purify the protein, a GSH-Sepharose 4B (Pharmacia) column was used. The BVR portion of the fusion protein was released by thrombin protease (Pharmacia) treatment. Purity was assessed by SDS-PAGE, stained as above.
Two-Dimensional Polyacrylamide Gel Electrophoresis (2D-IEF) and Western Blot Analysis of Phosphorylation:

2D-IEF was performed essentially as detailed previously (Huang et al., J. Biol. Chem. 264:7844-7849 (1989), which is hereby incorporated by reference). The first dimension separation of 3.5 μg of the reductase was carried out by isoelectric focusing (Righetti et al., J Chromatogr. 98:271-321 (1974), which is hereby incorporated by reference) using ampholytes with a 4-6.5 pH range. Electrofocusing was carried out for 17 h at 400 volts then for an additional hour at 800 volts. After focusing, the second dimension separation was carried out using 12.5% SDS polyacrylamide gel. The gels were subsequently probed for BVR and for phosphate associated with the protein using anti-rat BVR at 1/1000 dilution or a mixture (equal amounts, 2 μg/ml) of anti-phosphotyrosine, anti-phosphoserine, or anti-phosphothreonine antibodies. The secondary antibody was goat anti-rabbit horseradish peroxidase (Zymed) at a 1:1000 dilution. Detection was carried out using the ECL reagent kit (NEN) according to the manufacturer's instructions. Phosphorylation molecular weight markers were used (Zymed). Soybean trypsin inhibitor (pI 4.55), β-lactoglobin A (pI 5.13), bovine carbonic anhydrase (pI 5.85), and human carbonic anhydrase B (pI 6.57) were used as IEF standards. Immunoblotting was performed by the method of Towbin et al, Proc. Nat. Acad. Sci. USA 76:4350-4354 (1979), which is hereby incorporated by reference. Primary and secondary antibody treatments and staining for peroxidase with 4-chloro-1 -naphthol were performed as described previously (Huang et al., J. Biol. Chem. 264:7844-7849 (1989), which is hereby incorporated by reference).

Autophosphorylation of Biliverdin Reductase:

The ability of the reductase to phosphorylate itself was examined using the in blot method of Ferrell et al., Methods Enzymol. 200:430-435 (1991), which is hereby incorporated by reference. Following electrophoresis on 12.5% SDS-PAGE plus thioglycolate (0.002%), the protein was transferred to PVDF membrane (Millipore). The blot was denatured for I h at room temperature in 7 M guanidine-HCl (pH 8.3) containing 50 mM Tris-base, 50 mM dithiothreitol and 2 mM EDTA and the bound protein was allowed to renature overnight at 4° C. then incubated at room temperature for 4 h in freshly prepared labeling mix containing 10 μCi/ml [γ³²P]-ATP. The membrane first with 30 mM Tris-HCl (pH 7.4) then for 10 min each with 250 ml of the following: with 30 mM Tris-HCl (pH 7.4) twice; with the same buffer containing 0.5% (v/v) NP-40 once; with the Tris buffer twice; with 1 M KOH once; with the Tris buffer, twice . The membrane was blotted, dried, wrapped in plastic wrap, and exposed for autoradiography.

Measurement of Kinase Activity and Effect of Biliverdin Reductase on PKC Activity:

Kinase reactions were carried out based on the assay as described by Roskoski (Roskoski, Methods Enzymol. 99:3-6 (1983), which is hereby incorporated by reference) in a 50 μl volume in a reaction buffer consisting of 20 mM Tris HCl (pH 7.4) containing 10 mM MgCl₂, 50 mM unlabeled ATP, 5 μCi [γ³² P]-ATP and 1 mg/ml substrate. Substrates used were prepared as 10 mg/ml stock in 0.2 M Tris-HCl (pH 7.4) and stored at -20° C. The substrates utilized were MBP and histone III-S (Sigma) and E. coli expressed purified preparations of full length rat HO-1 and HO-2 (McCoubrey et al., J. Biol. Chem. 272:12568-12574 (1997), which is hereby incorporated by reference). The substrate/reaction buffer was incubated at 30° C. for 4 min prior to the addition of 5 μl of enzyme source (2 mg/ml BVR, 0.5 μg/ml PKC) or enzyme vehicle (GST-PBS containing 10% glycerol). Samples were mixed by aspiration and were then incubated for 8-15 min at 30° C. An aliquot (40 μl) from the reaction mixture was spotted onto a P81 (Whatman) filter, which was immediately immersed in 75 mM phosphoric acid for ≧5 min. Filters were washed 5× for 5 min each in 75 mM phosphoric acid and then 1× for 5 min with acetone before being air dried and counted by liquid scintillation counting in 10 ml ScintiVerse BD (Fisher). Background counts for samples containing substrate and vehicle were subtracted from those for substrate plus enzyme to determine net cpm. Activity was determined as ³²P counts incorporated into the acceptor protein per minute. For kinetic analysis of effects on PKC activity, BVR (final concentration 1.5 mg/ml˜50 μM), peptides (50 μM) or PBS was incubated at 30° C. with PKC (final concentration 0.5 μg/ml) for 15 min prior to addition of 5 μl to the kinase assay described above and using varying concentrations of MBP as substrate and ATP. Incubation time was 8 min at 30° C. The value obtained for PKC in the absence of BVR or peptides was considered to be 100%. For analysis of dose dependence of BVR or peptide on PKC activity, serial 2-fold dilutions were made in PBS and were mixed with a constant concentration of PKC prior to assay. For kinetic analysis, the substrate, MBP, was varied over the range of 0.25-2 mg/ml or ATP concentration was varied from 10-40 μM. Counts per minute for each sample were corrected for background using replicate samples, which did not receive PKC. Double reciprocal (Lineweaver-Burk) plots were generated by linear regression from the data points.

Overlay Assay for PKC Binding by Rat BVR:

The physical interaction of BVR with PKC was assessed using an overlay assay based on that described by Wolf and Sahyoun (J. Biol. Chem. 261:13327-13331 (1986), which is hereby incorporated by reference) with known modifications (Chapline et al., J. Biol. Chem. 268:6858-6861 (1993), which is hereby incorporated by reference). Purified rat liver BVR (2 μg) was subjected to SDS-PAGE and transferred to nitrocellulose membrane. The membrane was blocked, washed, and then overlaid with a solution containing 500 ng/ml of PKC (a mixture containing equal amounts of isozymes α, β and γ) (Promega) in TBS containing 10 mg/ml BSA, 20 μg/ml phosphatidylserine, 1 mM EGTA, 1.2 mM CaCl₂, 10 μg/ml leupeptin and 10 μg/ml aprotinin. After incubation for 1 h at room temperature, the blot was extensively washed with the PBS containing the same supplements as the overlay solution, but lacking the kinase mixture. The protein on the membrane was fixed by incubation in 0.5% formaldehyde in PBS and then reactive aldehyde groups were blocked by incubation in 2% glycine in TBS for 20 min at room temperature. The membrane was then washed 3 times with TBS and was cut in two, one half was subjected to Western blot analysis using a 1:1000 dilution of anti-PKC for 2 h at room temperature as the primary antibody, while the second half was probed with rabbit anti-rat BVR as detailed above for Western blotting. Rainbow molecular weight markers were used.

Results and Discussion

The existence of multiple charge and molecular weight variants of rat BVR has previously been described, which suggesting posttranslational covalent modification of the protein (Huang et al., J. Biol. Chem. 264:7844-7849 (1989), which is hereby incorporated by reference). To examine whether any of these variants result from phosphorylation of the reductase, the following experiment was performed.

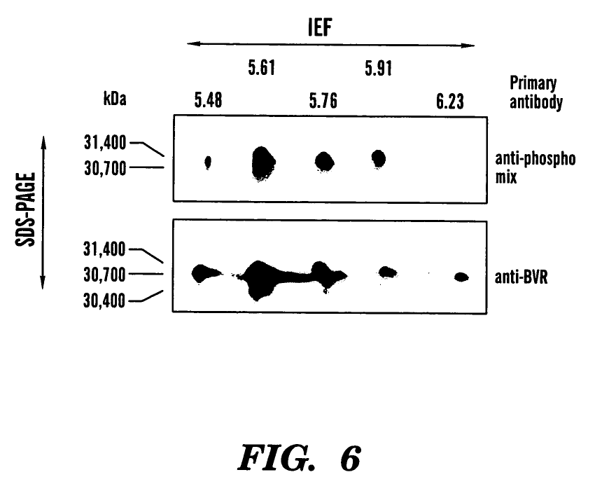

Replicate samples of the purified rat liver protein were subjected to 2D-IEF and were transferred to nitrocellulose. The membranes were then treated as Western blots employing as primary antibodies, either rat anti-BVR or anti-phospho mix as described above. The results shown in FIG. 6 indicate that the pattern of mobility of the phosphorylated variants (top panel) is, for the most part, similar to that of variants detected by antibody to BVR (bottom panel). Based on relative intensity of staining for phospho-amino acids and protein, it appeared, however, that the relative extent of phosphorylation of the variants differ. For instance, when probed with the anti-phospho mix, the 30,400 kDa variant with a pI of 5.61 consisting of two molecular weight populations (30.4 and 31.4 kDa) was not resolved and the 30,700 kDa, pI 6.23 variant was at the limit of detection. Previous studies have established that all BVR variants are active oxidoreductases (Huang et al., J. Biol. Chem. 264:7844-7849 (1989), which is hereby incorporated by reference). Therefore, these findings raise the possibility that activity can be carried out in the absence of phosphorylation.

The most commonly occurring covalent modification of amino acids with phosphate involves serine and threonine residues; tyrosine phosphorylation is, in comparison, rare (Kamps et al., Nature 310:589-592 (1984); Sternberg et al., FEBS Lett. 175:387-392 (1984); Hunter et al., Ann. Rev. Biochem. 54:897-930 (1985); Hanks et al., Science 241:42-52 (1988); Schlessinger, Trend. Biochem. Sci. 13:443-447 (1988), which are hereby incorporated by reference). To examine which phosphoamino acids are present in rat BVR, Western blot analysis was carried out using the same purified rat liver enzyme preparation probed with anti-phospho mix (defined above) or individually with anti-phosphotyrosine, anti-phosphothreonine or anti-phosphoserine. As expected, and shown in FIG. 6, an anti-phospho mix immunoreactive band, with an increase in intensity as a function of BVR concentration, is detected. Blots were probed with the individual antibodies, showing that rat BVR is an unusual phosphoprotein and is esterified on tyrosine (panel b), serine (panel c), and threonine (panel d) residues.

A large number of proteins are phosphorylated, but only a small fraction are reversibly phosphorylated. Therefore, it was examined whether BVR is capable of autophosphorylation. To assess this capability, SDS-PAGE was performed using the rat liver purified enzyme preparation and the protein was transferred to PVDF membrane. The membrane bound protein was subjected to denaturation and renaturation and subsequently incubated with [γ³²P]-ATP (Ferrell et al., Methods Enzymol. 200:430-435 (1991), which is hereby incorporated by reference). This protocol has been used to demonstrate autophosphorylation of proteins. The results of this analysis, shown in FIG. 2, demonstrated that the reductase is indeed able to phosphorylate itself (panel a). Panel b shows a stained SDS-PAGE of the same BVR preparation indicating that the signal observed in panel a is, in fact, due to phosphotransferase activity of the reductase.

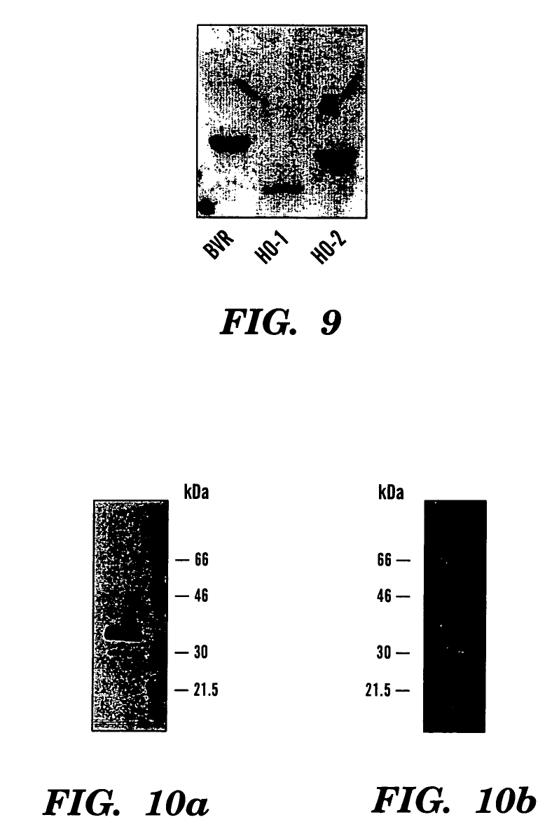

Next, the ability of BVR to phosphorylate exogeneous substrates was assessed in an in vitro assay measuring incorporation of ³²P into the commonly used γ-phosphate, MBP, and hi stone III-S in heme oxygenase (HO) isozymes-1 and -2, which are immediately upstream of BVR in the heme degradation pathway. Assays were carried out in the presence or absence of diacylglycerol (“DAG”) and calcium. Incorporated ³²p counts were corrected for background based on replicate reactions carried out in the absence of BVR. Reproducibly low levels of phosphate (˜4,000 cpm/min) were transferred by BVR to MBP. Histone III-S was a poor acceptor protein (˜1,000 cpm/min). The phosphotransferase activity of BVR was calcium and phospholipid (DAG)-dependent. HO-1 and HO-2 were tested as substrates. Although both were found to be phosphoproteins, as shown in FIG. 9, they were not substrates for BVR, nor were they autophosphorylated. The unimpressive phosphotransferase activity with MBP and histone suggests that those proteins are not the physiological acceptor proteins. The identity of the natural substrate(s), if any, in the cell remains to be established. Nonetheless, because phosphotransferase activity, using HO-1 and HO-2 as substrates, was below the level of detection of the assay, it is reasonable to suggest that the phosphotransferase activity of BVR is not relevant to its function in the heme degradation pathway.

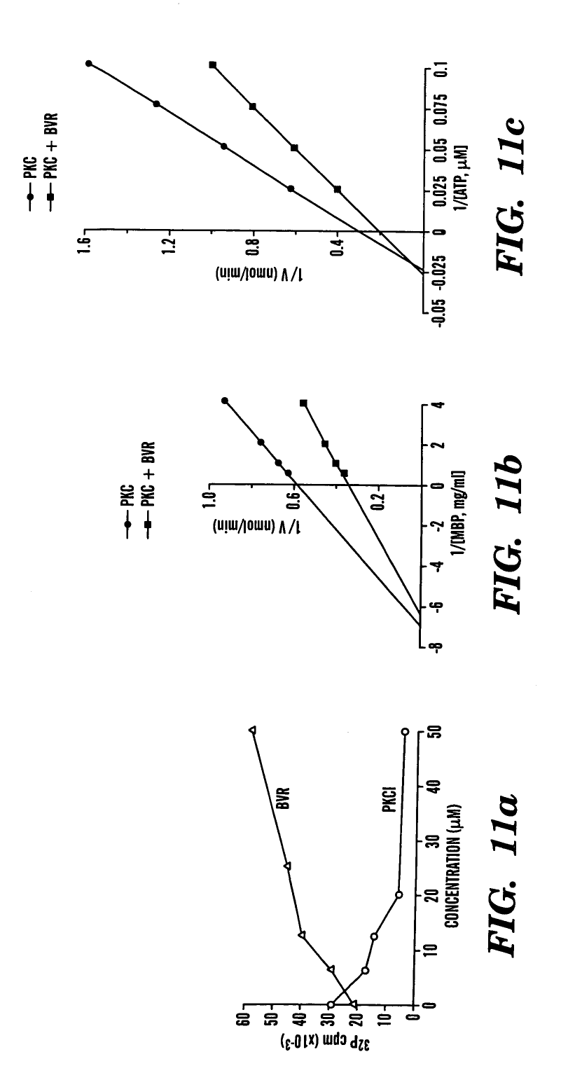

The PKC isozymes, 12 forms of which have been described to date, are a major family of serine/threonine kinases. Therefore, it was examined whether BVR is upstream or downstream of the PKC pathway. First, however, because of the high degree of similarity between certain segments of BVR and known PKC-interactive peptides, experiments were conducted to examine the ability of rat BVR to interact with PKC using the overlay technique. This technique has been used to identify PKC substrates (Wierenga et al., Nature 302:842-844 (1983), which is hereby incorporated by reference). As shown in FIG. 10, the antiserum to α, β, and γ isozymes detected the presence of PKC (panel a) with the same mobility as detected for the reductase in a blot in which the primary antibody was directed to BVR (panel b). This finding defines BVR as a PKC-interactive protein. The 30 kD band noted in both panels is truncated BVR and is observed whenever the protein undergoes extensive manipulation.

Having established that the reductase and PKC interacted with each other, it was then determined whether this interaction had an effect on PKC activity. For this, PKC was incubated at 30° C. for 15 min with buffer or varying concentrations of BVR prior to addition to the PKC assay system, using MBP as the substrate. As a control, a known peptide inhibitor of PKC (“PKCI”) (SEQ. ID. No. 29) was used in a second set of reactions over the same range of concentrations as BVR. The results of this experiment are presented in FIG. 11 A. The PKCI peptide showed the anticipated dose-dependent inhibition of activity. Surprisingly, incorporation of ³²P into MBP was increased in a dose-dependent manner in the presence of BVR. This increase in activity was not due to phosphorylation of BVR by PKC, or the reciprocal, as it was dependent on the presence of the substrate, MBP. The basis for stimulation of PKC was examined with respect to MBP and ATP kinetics (FIGS. 11B-C, respectively). The kinetic data indicate that both substrate and interaction with PKC were effected by BVR. In the presence of BVR, Vₘₐₓ of the reaction, both in respect to the MBP and ATP, was increased from 3.3 to 4.9 and 1.7 to 2.9 pmol/min, respectively. The effect on Kₘ of the reaction was not as prominent with substrate Kₘ increasing from 0.145 to 0.16 upon addition of BVR to the reaction. The effect on Kₘ of the reaction with respect to ATP was negligible.

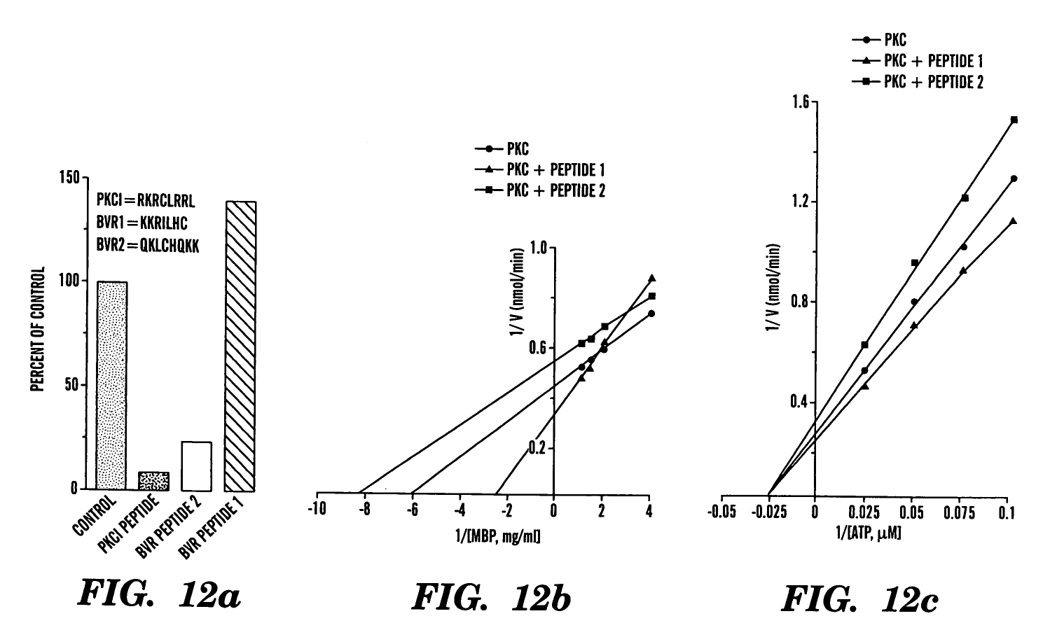

In the primary structure of BVR, clusters of residues are present that, based on their charge character, could potentially interact with PKC. Two rBVR derived peptides, KKRIMHC (rBVR amino acids 274-280, SEQ. ID. No. 18) and QKLCHQKK (rBVR amino acids 288-295, SEQ. ID. No. 19), as well as two hBVR derived peptides, KKRILHC (hBVR amino acids 275-281, SEQ. ID. No. 34) and QKYCCSRK (HBVR amino acids 289-296, SEQ. ID. No. 35), have partial homology in composition to PKCI peptide, RKRCLRRL (SEQ. ID. No.29). Also, the net charge of the BVR derived peptides is quite similar to that of the PKCI peptide. Thus, it seemed plausible that those peptides might alter PKC activity. When tested in a PKC assay (FIG. 12A), peptide 1 stimulated PKC activity, whereas peptide 2 inhibited PKC activity by more than 75%. This was comparable to the inhibition produced by the PKCI peptide itself, which produced a 90% inhibition of PKC when added to the PKC assay system at the same concentration as BVR peptide 2. Although opposite in direction to that seen for the intact enzyme, this inhibition of PKC by the peptide indicates an interaction between the peptide and the kinase. Subsequently, the kinetics of the interaction of the two peptides with PKC was examined with respect to substrate (FIG. 12B) and cofactor (FIG. 12C). Analysis of data indicate that both peptides altered the Kₘ, as well as the Vₘₐₓ of the reaction with substrate. Inhibition of the reaction by peptide 2, with respect to substrate concentration, was of the scarce uncompetitive nature wherein both Vₘₐₓ and Kₘ were decreased from 2.25 pmol/min and 0.165 mg/ml to 1.82 pmol/min and 0.12 mg/ml. The interaction with peptide 1 exhibited mixed kinetics indicating that at lower concentrations the peptide has antagonist effect on substrate binding and at higher concentrations it stimulates PKC activity. Vₘₐₓ of the reaction was increased by this peptide to 2.92 pmol/min and Kₘ for the substrate increased to 0.39 mg/ml. With respect to the cofactor, both peptides effected Vₘₐₓ of the reaction. Vₘₐₓ in the presence of peptide 1 was 4.2 and 3.12 for peptide 2 compared to 3.81 pmol/min for the control reaction. The findings collectively indicate that interaction of BVR and peptides corresponding to its carboxy terminal sequence interact with PKC to alter its kinetic behavior.

Thus, the experimental evidence defines rBVR as a kinase and, most interestingly, demonstrates its capacity to stimulate PKC. The reductase is rather unusual in being a serine-, threonine-, as well as a tyrosine-phosphoprotein. Notably, tyrosine phosphorylation, accounts for less than 0.1% of the sum of serine/threonine phosphoproteins (Wierenga et al., Nature 302:842-844 (1983); Martensen et al., Methods Enzymol. 99:402-405 (1983); Kamps et al., Nature 310:589-592 (1984); Sternberg et al., FEBS Lett. 175:387-392 (1984); Hunter et al., Ann. Rev. Biochem. 54:897-930 (1985); Hanks et al., “The Protein Kinase Family: Conserved Features and Deduced Phylogeny of the Catalytic Domains,” Science 241:42-52 (1988); Schlessinger, “Signal Transduction by Allosteric Receptor Oligomerization,” Trend. Biochem. Sci. 13:443-447 (1988); Taylor et al., FASEB J. 9:1255-1266 (1995), which are hereby incorporated by reference). While these experimental results do not identify which of the 5 tyrosine, 7 threonine, and 24 serine residues in BVR are substrates for phosphotransferase activity of BVR, it is clearly demonstrated that BVR has kinase activity with general kinase substrates, MBP and histone III-S, and that its kinase activity is not directed towards the upstream components of the heme catabolic pathway.

In its primary structure, BVR has many conserved features of protein kinases. These include the Gly¹⁵.Xaa.Gly.Xaa.Xaa.Gly²⁰ (SEQ. ID. No. 7) consensus motif in the N terminus of the protein. This motif is conserved in the cyclic nucleotide regulated protein kinase family (Hanks et al., Science 241:42-52 (1988); Schlessinger, Trend. Biochem. Sci. 13:443-447 (1988); Edelman et al., Ann. Rev. Biochem. 56:567-613 (1987); Yarden et al., Annu. Rev. Biochem. 57:443-478 (1988), which are hereby incorporated by reference). Based on a model of the ATP binding site (Taylor et al., FASEB J. 9:1255-1266 (1995), which is hereby incorporated by reference) in protein kinases, the Gly.Xaa.Gly.Xaa.Xaa.Gly (SEQ. ID. No. 7) consensus motif is found in subdomain I, near the amino terminus catalytic domain (Sternberg et al., FEBS Lett. 175:387-392 (1984); Hunter et al., Ann. Rev. Biochem. 54:897-930 (1985); Hanks et al., Science 241:42-52 (1988), which are hereby incorporated by reference). In the reductase, the consensus is immediately flanked upstream by a stretch of 4 valines and a cluster of basic residues (K.R.K.) and a valine residue is located 2 positions on the carboxy terminal side of the consensus. The four valine residues is a hydrophobic domain conserved among membrane associated proteins. In a model of the ATP binding site, a nearly invariant valine residue is located in subdomain I, 2 positions downstream of the consensus (Sternberg et al., FEBS Lett. 175:387-392 (1984); Hanks et al., Science 241:42-52 (1988), which is hereby incorporated by reference). The valine may function in positioning of the glycine residues. Basic residues are utilized in serine/threonine kinases as specificity determinants (Kemp et al., Proc. Natl. Acad. Sci. USA 80:7471-7475 (1983), which is hereby incorporated by reference).

Clusters of charged amino acids are important for various aspects of kinase activity; they are found in proteins themselves, in substrates for some protein kinases (Cohen, Curr. Top. Cell Reg. 14:117-196 (1978); Kamps et al., Mol. Cell. Biol. 6:751-757 (1986), which is hereby incorporated by reference), as well as in inhibitors of kinase activity (House et al., Science 238:1726-1728 (1987), which is hereby incorporated by reference). Indeed, polylysine and polyarginine are potent inhibitors of PKC-mediated substrate phosphorylation (House et al., Science 238:1726-1728 (1987), which is hereby incorporated by reference). Two basic amino acid clusters are found at the C terminus of BVR: K²⁷⁴KRIMHC (peptide 1, SEQ. ID. No. 18) and Q²⁸⁸KLCHQKK (peptide 2, SEQ. ID. No. 19) in rBVR and K²⁷⁵KRILHC (SEQ. ID. No. 34) and Q²⁸⁹KYCCSRK (SEQ. ID. No. 35) in HBVR. These overlap the putative zinc-binding site of BVR (Maines et al., Eur. J. Biochem. 235:372-381 (1996), which is hereby incorporated by reference) and closely resemble the CHQKR motif found in serine/threonine protein kinases. It is believed, based on kinetic data, that these regions of the reductase take part in BVR interaction with PKC. The finding that both peptides influence Kₘ and Vₘₐₓ of substrate kinetics, suggests their interaction with PKC causes conformational changes in the catalytic domain of the kinase. In many protein kinases, the regulatory domain contains a pseudosubstrate, which binds to the catalytic site and prevents access of the “true” substrate to catalytic domain (Kemp et al., Proc. Natl. Acad. Sci. USA 80:7471-7475 (1983), which is hereby incorporated by reference). It is possible that binding of BVR and, at higher concentrations, binding of peptide 1 to the regulatory domain relieves this internal inhibitory effect. As noted above, phosphorylation of PKC is not increased in the presence of BVR or peptide 1. Therefore, this is not the mechanism by which BVR activates PKC. The interaction with peptide 2 with PKC displays the rare noncompetitive inhibition kinetics and data suggest conformational change in the kinase structure. These activities of peptide 1 and peptide 2 are also present for their homologs in hBVR (i.e., SEQ. ID. Nos. 34 and 35).

As is known, PKC constitute a family of at least about 11 related proteins with limited substrate specificity that in the cell mediate isozyme-specific functions (Newton, J. Biol. Chem. 270:28495-28498 (1995), which is hereby incorporated by reference). The specificity of function depends on interaction of the isoenzyme with specific targeting proteins that serve as an anchor (or scaffold or racks) (Mochly-Rosen, Science 268:247-251 (1995); Klauck, Science 271:1589-1592 (1996); Newton, Curr. Opin. Cell Biol. 9:161-167 (1997); Mochly-Rosen et al., FASEB J. 12:35-42 (1998), which are hereby incorporated by reference). Not all proteins that bind to kinases involved in signal pathways are known. The present study defines BVR as a PKC binding protein; and that interaction can occur with a mix of α, β and γ PKC. Because of these interactions, it is possible that the reductase could be a significant modulator of signal transduction pathways.

In the cell, the kinase activity of BVR may not be directed only toward itself. Thus, it may participate in phosphorylation and/or activation of other substrates. BVR kinase activity may have the potential of regulating activity in the cell, as is in the case of all kinases. Moreover, in the assay system used for determining autophosphorylation, there were no other proteins or kinases present, therefore, it is obvious that phosphorylation is carried out by BVR itself. It is possible that BVR may also phosphorylate other proteins by itself in cells. In the primary structure of BVR, motif search reveals several sites that potentially can be phosphorylated by various protein kinases.

### Example 4—Role of Biliverdin Reductase in Human Renal Cell Carcinoma

Materials and Methods

Chemicals:

Oligo (dt)—cellulose, Salmon testis DNA, paraformaldehyde, dextran sulfate, Triton X-100, diaminobenzimide, and 4-chloro-3-napthol were obtained from Sigma Chemical Company (St. Louis, Mo.). Goat anti-rabbit γ-globulin, rabbit peroxidase-antiperoxidase, and goat anti-rabbit γ-globulin conjugated to horseradish peroxidase were obtained from Organon Teknika-Cappel Corporation (Westchester, Pa.). Nytran filters and nitrocellulose (0.2 μm pore size) were from Schleicher and Schuell (Keene, N.H.).

Tissue Samples:

Kidney tissue specimens were obtained from 3 patients who had radical nephrectomy performed for stage t2 clear cell renal carcinoma. Tumor size ranged from 3 to 5 mm³. Normal kidneys were obtained from a brain dead patient. Each sample was divided for use in immunohistochemistry or biochemical analyses. Portions of kidney from patients with renal carcinoma were further divided into visible tumor and portions without visible tumor. Tumors are pooled and used for molecular and biochemical analyses. Immunostaining was also carried out using 10 additional formalin fixed specimens of renal cell carcinoma.

RNA Preparation and Northern Blot Analysis:

Total RNA was prepared and used for isolation of polyadenylated RNA by oligo (dT)-cellulose chromatography (Kingston, In Current Protocols in Molecular Biology, (Ausubel et al., eds), Wiley and Sons, New York, 451 (1987), which is hereby incorporated by reference). Formaldehyde-denatured poly(A)+RNA was fractionated on a 1.2% agarose gel, transferred to Nytran and the filter was subsequently baked in vacuo for 1.5-2 h at 80° C. Prehybridization, hybridization of the appropriate ³²P-labeled cDNA and posthybridization treatment of the filter was performed essentially as described previously (Maines et al., Urology 47:727 (1996), which is hereby incorporated by reference). Hybridization probes, a PCR product consisting of nucleotides +401 to +926 of biliverdin reductase cDNA (Maines et al., Eur. J. Biochem. 235:372 (1996), which is hereby incorporated by reference) and mouse α-actin cDNA probe (Minty et al., J. Biol. Chem. 256:1008 (1981), which is hereby incorporated by reference), were labeled according to the manufacturer's instructions with [α-³²P] dCTP by the random priming method, and further purified by spin chromatography (Sambrook et al., Molecular Cloning: A Laboratory Manual, Cold Spring Harbor Laboratory, Cold Spring Harbor, N.Y. (1982), which is hereby incorporated by reference). mRNA levels were quantitated by laser densitometry.

Antibody Production and Western Blot Analysis of Biliverdin Reductase:

Biliverdin reductase was purified from human kidney and used for antibody production in White New Zealand rabbits (Maines et al., Arch. Biochem. Biophys. 300:320 (1993), which is hereby incorporated by reference). Kidney cytosol was obtained as before (Maines et al., Arch. Biochem. Biophys. 300:320 (1993), which is hereby incorporated by reference) and subjected to SDS-polyacrylamide gel electrophoresis (Laemmli, Nature 227:680 (1970), which is hereby incorporated by reference) under denaturing conditions, and transferred to nitrocellulose membrane. The filter was subsequently subjected to Western blot analysis according to the procedure of Towbin et al (Proc. Nat. Acad. Sci. USA 76:4350 (1979), which is hereby incorporated by reference) as modified by Huang et al. (J. Biol. Chem. 264:7844 (1989), which is hereby incorporated by reference). Protein was visualized using human kidney biliverdin reductase as the primary antibody.

Assay Procedure:

Protein was measured by the method of Lowry et al. (J. Biol. Chem. 193:265 (1951), which is hereby incorporated by reference). Bovine serum albumin was used as the protein standard. Measurements of enzyme activity were performed as detailed before (Kutty et al., J. Biol. Chem. 256:3956 (1981), which is hereby incorporated by reference). The reaction was initiated by the addition of 120 μl of either 10 mM NADH at pH 6.7 or 1 mM NADPH at pH 8.7 to 1.2 ml test reaction mixture at the corresponding pH. The conversion of biliverdin to bilirubin was measured from increased absorption at 450 nm at 30° C.

Immunohistochemistry of Biliverdin Reductase:

Mouse monoclonal antibodies to human T cell CD3, human leukocyte CD45 (70536879R) and human macrophage CD68 were purchased from Zymed (San Francisco, Calif.). Removed kidneys were placed on ice and after histopathology, sections were placed in 0.1 M phosphate buffer (pH 7.2) containing 4% (w/v) paraformaldehyde. Kidney was post-fixed for 16 h (at 4° C.) prior to sequential dehydration and then paraffin embedding. Ten-μm-thick sections were obtained. Biliverdin reductase immunohistochemistry was carried out using peroxidase-antiperoxidase procedure as previously described (Towbin et al., Proc. Nat. Acad. Sci. USA 76:4350 (1979), which is hereby incorporated by reference) with 1:1000 dilution of primary antibody in 0.1 M phosphate buffer containing 0.3% (v/v) Triton X-100 and 10% (v/v) normal goat serum for 4 days at 4° C. Endogenous peroxidase activity of tissue was inhibited by treatment with 0.1 M phosphate buffer containing 3% (v/v) hydrogen peroxide and 10% (v/v) methanol for 8 min prior to incubation with primary antibody. When primary biliverdin reductase was omitted, staining was absent. CD3, CD45 and CD68 immunostaining were carried out using 1/100 dilution of antisera and alkaline phosphatase as chromogen (AP-Blue). For double immunostaining, tissue was stained first followed by peroxidase staining using AEC as chromogen for biliverdin reductase staining. Counter staining with hematoxylin was carried out (Davis et al., J. Urol. 142:884-888 (1989), which is hereby incorporated by reference).

Results and Discussion

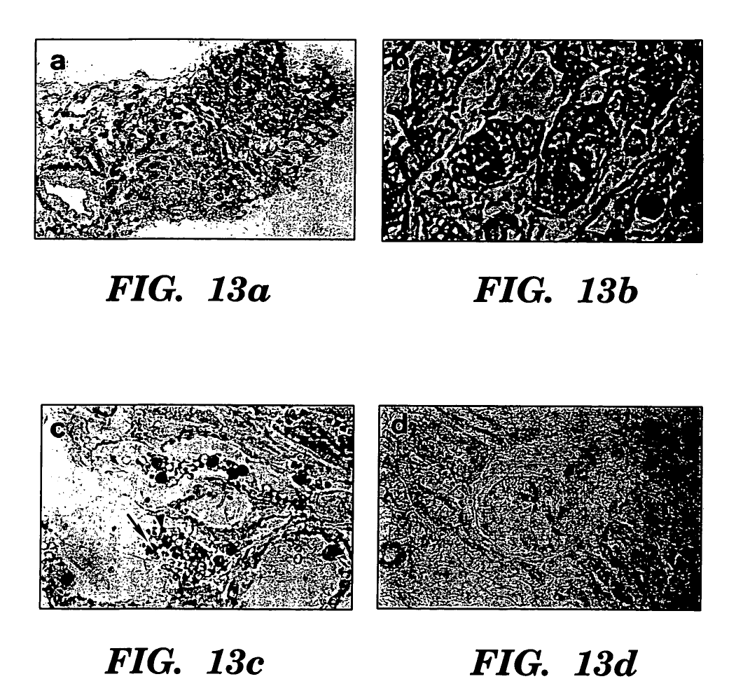

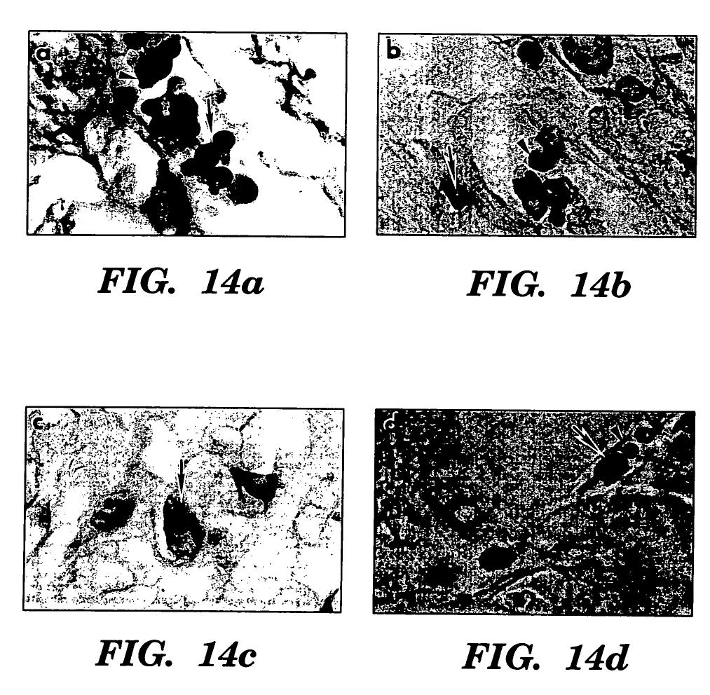

FIG. 13A shows biliverdin reductase immunostaining in kidney tumor and FIG. 13B shows staining of nests of tumor cells infiltrating the stroma at higher magnification. As noted, overall a strikingly intense immunoreactivity was detected in the tumor tissue. The intensity of staining, however, was not uniform throughout the tumor and varied in different areas of the tumor as among different formalin-stained specimens. Nonetheless, the increase in staining of the tumor over the surrounding tissue was common to all specimens. For comparison, staining of the area surrounding the tumor is shown in FIG. 13C and staining of the normal kidney is presented in FIG. 13D. As shown in FIG. 13C, tissue surrounding the tumor displayed intense immunostaining for the reductase which was detected in the cytoplasm of intravascular neutrophils; the erythrocytes did not stain for the reductase. In contrast to the tumor cells, staining for the reductase was rather unremarkable in normal tissue where weak cytoplasmic staining of apical epithelial portion of tubules was observed. Weak staining for the reductase was also observed in the normal tissue glomerulus with a few neutrophils in glomerular capillaries. Biliverdin reductase staining was present in infiltrating leukocytes, cells including macrophages and neutrophils as detected by double staining with CD68 (FIG. 14A); with T cells as detected by CD3 double immunostaining (FIG. 14B) and with lymphocytes as detected by double staining with CD45 (FIG. 14C). As shown in FIG. 14D, the reductase immunostaining was also present in non-infiltrative leukocytes. The image depicts staining of leukocytes in a vessel in normal kidney tissue. Again, as noted, erythrocytes do not stain for the reductase.

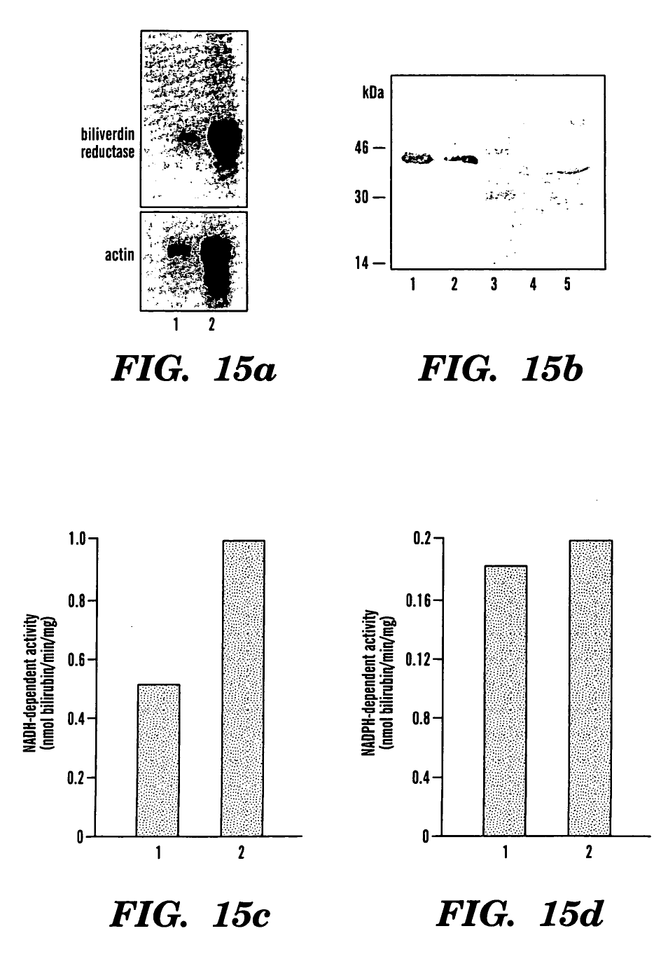

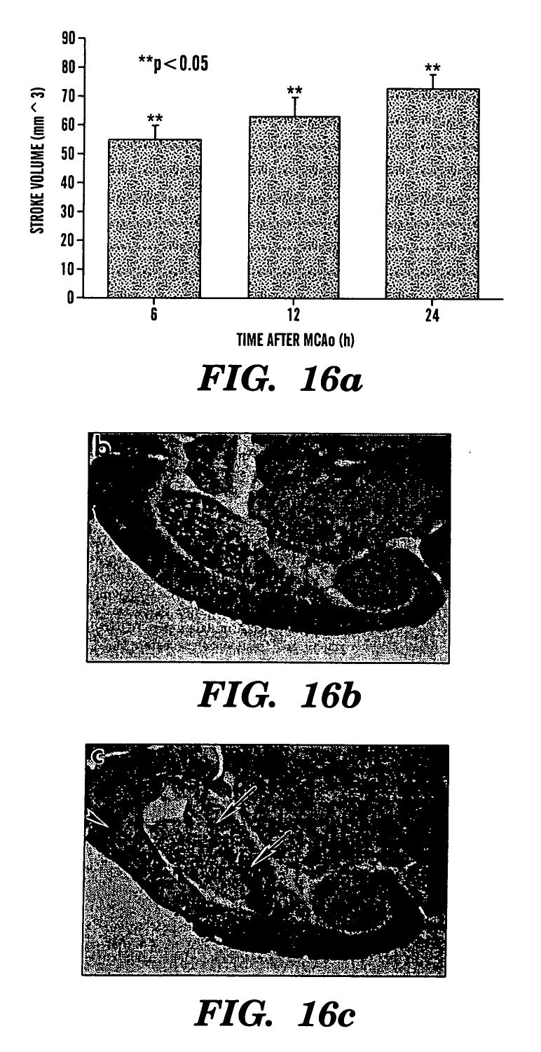

The increase in biliverdin reductase immunostaining was related to increases in levels of the reductase transcript and protein, as suggested by Northern blot analysis of the mRNA (FIG. 15A) and by Western blot analysis of protein (FIG. 15B). To examine whether the increased expression of the reductase leads to an increase in an active form of the enzyme, biliverdin reductase activity was measured at pH 6.7 and 8.7 using NADH and NADPH as cofactors, respectively (FIG. 15C). It was found that NADH-dependent activity was increased by nearly 70%. This was contrasted by the near absence of a change in NADPH-dependent activity.

The above data illustrates a pattern of biliverdin reductase expression in human tissue, and in response to pathological conditions. Although, in recent years a number of regulatory functions have been ascribed to both the substrate, biliverdin, as well as the product of enzyme activity, bilirubin (Nakagami et al., Microbial Immunol. 36:381 (1992); Stocker et al., Science 235:1043 (1987); Stocker et al., Proc. Nat. Acad. Sci. USA 84:8130 (1987); Marks et al., Trends Pharmacol. Sci. 12:185 (1991); Willis et al., Nature Med. 2:87 (1996); Maines, Ann. Rev. Pharmacol. Toxicol. 37:517 (1997); Woo et al., Transplant Immunol. 6:84-93 (1998), which are hereby incorporated by reference), the enzyme has been considered solely in the context of its reductase activity in converting heme oxygenase activity product to bile pigments. Hence, the fact that in the process of reducing biliverdin it oxidizes NADH and is a NADH dehydrogenase has gone unnoticed.

The above data shows upregulation of the reductase in renal cell carcinoma, both at the transcript and protein levels. It is relevant to note that previous studies have shown increased expression of the stress-inducible form of heme oxygenase, HO-1 (Maines, Ann. Rev. Pharmacol. Toxicol. 37:517 (1997), which is hereby incorporated by reference), both in prostate cancer tumors (Ewing et al., J. Neurochem. 61:1015 (1993), which is hereby incorporated by reference) and in renal carcinoma (Maines, Ann. Rev. Pharmacol. Toxicol. 37:517 (1997), which is hereby incorporated by reference). In turn, increase in HO-1 expression has been shown to cause immunosuppression and modulation of inflammatory response (Willis et al., Nature Med. 2:87 (1996); Woo et al., Transplant Immunol. 6:84-93 (1998), which are hereby incorporated by reference), which have been suspected to involve the function of heme oxidation products, including biliverdin and bilirubin (Willis et al., Nature Med. 2:87 (1996); Woo et al., Transplant Immunol. 6:84-93 (1998), which are hereby incorporated by reference). While the mechanisms by which cellular transformation leads to upregulation of the reductase and the molecular basis for the increase only in NADH-dependent activity are not yet known, it is reasonable to suspect alterations in post translational modification of the protein (Huang et al., J. Biol. Chem. 264:7844 (1989), which is hereby incorporated by reference). Nonetheless, based on the unique catalytic properties of the enzyme with respect to its dual pH/cofactor requirements (Kutty et al., J. Biol. Chem. 256:3956 (1981); Huang et al., J. Biol. Chem. 264:7844 (1989), which are hereby incorporated by reference), and the observed single dimensional increase in enzyme activity with NADH in tumor cells may, in context NADH dehydrogenase activity, have relevance to both the survival of the tumor cell as well as to the host tissue defense.

Without being bound by theory, it is believed that the benefit offered to the tumor cell would include local increased production of bilirubin with its known antioxidant and immune function modulating activity. Also, by lowering levels of NADH, capacity of the cell to mediate NADH-driven iron-mediated reactions would be diminished, thus blocking production of free radicals. This is an analogy to NADH-dehydrogenase, the induction of which has been suggested to result in decreased levels of NADH and reduced levels of cellular redox cycling constituents (Woo et al., Transplant Immunol. 6:84-93 (1998), which is hereby incorporated by reference). Moreover, the fact that biliverdin reductase is a zinc metalloprotein, a class of protein known for possible regulatory activity in cells, means that increased expression of biliverdin reductase may be a significant event apart from bilirubin production.

A plausible extension of increased dehydrogenase activity would predict tumor cell death. Utilization of NADH generates NAD; and, ATP must be utilized for regeneration of the reduced nucleotide. The significance of this process can be viewed in terms of its synergism with cell killing activity of poly(ADP-ribose) polymerase. The polymerase is a chromatin bound enzyme, that cleaves NAD at the N-gyrosylic bond between ribose and the nitrotinamide (Goodman et al., Proc. Soc. Exper. Biol. Med. 214:54 (1997); Linn, Drug Metabl. Dispo. 30:313 (1988); Althaus et al., Mol. Biol. Biochem. Biophys. 37:1 (1987), which are hereby incorporated by reference). Activation of the polymerase is triggered by cell exposure to DNA damaging stimuli (Berger, Radiat. Res. 101:4 (1985); Eliasson et al., Nat. Med. 3:1089 (1997); Endres et al., J. Cereb. Blood Flow Metab. 17:1143 (1997), which are hereby incorporated by reference). Utilization of NAD by the polymerase and consequent lowering of cellular ATP pools needed for synthesis of the nucleotide are believed to account for rapid cell death before DNA repair occurs (Althaus et al., Mol. Biol. Biochem. Biophys. 37:1 (1987), which is hereby incorporated by reference). Therefore, the concerted activity of the reductase and the polymerase to deplete the cell of ATP could be expected to lead to tumor cell death.

Another intriguing finding is the substantial biliverdin reductase staining in a variety of white cells in the local tumor environment as well as in some of the white cells within the microvasculature of normal kidney. This would suggest that prominent reductase expression in leukocytes in perhaps independent of tumor infiltration. Heme metabolism utilizing heme oxygenase and biliverdin reductase within white cells would be expected given the presence of hemoproteins such as cytochromes, catalase and peroxidase. More difficult to determine, however, would be whether the intracellular biliverdin reductase expression either directly or via bilirubin production may be modulating the activity of the white cells in the presence of tumor antigen.

### Example 5—Role of Biliverdin Reductase in Preventing Neuronal Cell Death Following Stroke/Ischemic Event

Materials and Methods

Animals and Materials:

Inbred DNX mice of the same genetic background were obtained from DNX Labs (Princeton, N.J.) and maintained in a quarantined environment with free access to food and water. The National Institute of Health Guide for the Care and Use of Laboratory Animals was strictly followed during all in vivo experiments. The present study used 72 adult mice (25-36 g b.wt), of which 23 mice were used for assessment of stroke volume. For stroke studies, only male mice were used. Surgical instruments were purchased from Fine Science Tools (FST, Foster City, Calif.). Fluotec-3 anesthesia apparatus was obtained from Colonial Medical, (Amherst, N.H.) and homeothermic blanket with YSI thermocouple were from Yellow Springs, (Yellow Springs, Ohio). Portable intensive care system equipped with warmers, humidifier, nebulizer, and oxygen mixer was purchased from ThermoCare (Incline Village, Nev.).

Reagents for in situ hybridization were purchased from Sigma and were molecular biology ultra-pure grades. These included 20×SSC buffer concentrate, 50× concentrated Denhardt's solution, Tri-EDTA buffer 100× concentrate, Tris base, Magnesium chloride, formamide, mixed bed resin, N,N-dimethylformamide, bovine serum albumin, sodium acetate, triethanolamine hydrochloride, DMSO, paraformaldehyde, nitro blue tetrazolium (NBT), 5-bromo-4-chloro-3-indolylphosphate (BCIP), and levamisole. Digoxigenin-11-dUTP, dNTP labeling mix (dATP, dGTP, dCTP, dTTP), and antidigoxigenin-AP (alkaline phosphatase conjugate, Fab fragments) antibody were from Boehringer Mannheim (Mannheim, Germany). Proteinase K, fish sperm DNA, ethidium bromide and Taq polymerase were from Amersham Life Sciences (US Biochemical, Cleveland, Ohio) and QIAquick PCR purification kit (QIAQuick) was from QIAgen (Santa Clarita, Calif.). Graded ethanols, xylene, chloroform, 2-methylbutane and concentrated HCl were from VWR (Rochester, N.Y.). RNase AWAY (Molecular Bio-Products, San Diego, Calif.). Suppliers of antibodies and histochemical reagents are specified in appropriate sections. Biochemicals were purchased from Sigma and Aldrich Chemical, and were of the highest purity commercially available. Nytran sheets were purchased from Schleicher and Schuell (Keene, N.H.). β-³²P Deoxycytidine 5′-triphosphate (dCTP) was purchased from Amersham (Arlington Heights, Ill.).

Induction of Focal Cerebral Ischemia:

Adult mice were exposed to 3% halothane induction anesthesia administered by Fluotec 3 vaporizer, and were left spontaneously breathing 2% halothane in air by means of a nose cone. Middle cerebral artery (“MCA”) occlusion (“MCAo”) was induced as previously described (Panahian et al., J. Neurochem. 72:1187-1203 (1999), which is hereby incorporated by reference). MCA was occluded above the level of the olfactory tract, thus lenticulostriate arteries (“LSA”) were left intact. The method of MCA occlusion was a traditional intracranial technique performed using subtemporal approach without occlusion of the ipsilateral common carotid artery. Body temperature was controlled and maintained at 37° C. by means of a homeothermic blanket and YSI thermocouple. Other systemic physiological parameters were reported by us previously for this model (Panahian et al., J. Neurochem. 72:1187-1203 (1999), which is hereby incorporated by reference). The studied animals did not sustain blood loss, and all surgeries were completed within 7-10 min. There were no mortalities and all animals were included in the study. For determination of volume of stroke, animals were killed at 6 (n=8), 12 (n=8) or 24 h (n=7) after induction of ischemia. Behavioral assessment of mice after stroke was carried out as reported previously (Panahian et al., J. Neurochem. 72:1187-1203 (1999), which is hereby incorporated by reference).

Assessment of Stroke Volume:

A 166 MHz MMX Pentium computer equipped with FlashPoint 128 video card and Hitachi digital KP-D50 color CCD camera running ImagePro v. 3.1 image analysis software was used to acquire images of brains under 10× magnification of BH-2 Olympus microscope (equipped with 10×/20 eyepieces) for assessment of stroke volume. Plan Apochromatic ×40 objectives were used to capture images for cell related morphometrical quantitation. Pyramidal cortical neurons in layers 3 and 5 were examined and quantified as previously reported (Sieber et al., Stroke 26:2091-2096 (1995), which is hereby incorporated by reference). The number of neurons per square millimeter was determined blindly for each region of interest in both ischemic and control brain specimens. Stroke volume (mm³) was derived by numerical integration of consecutive hematoxylin and eosin (H & E) stained 15 μm thick coronal frozen sections sampled at regular intervals using the following formula:

$$Vs ( {mm}^{3} ) = Σ ( Areas of hemispheric lesions ) \times Distance ( between sampled histological sections )$$

Swanson technique, which corrects for infarct volume based on adjustments for hemispheric edema, was used as reported previously (Panahian et al., J. Neurochem. 72:1187-1203 (1999); Swanson et al., J. Cereb. Blood Flow Metab. 10:290-293 (1990), which are hereby incorporated by reference). Serial coronal sections of stroked brains were cut using Leica 1800 cryostat, stained for H & E, and used for calculation of infarct volumes.

Immunocytochemical Protocol for Detection of BVR:

A total of 14 mice (n=4 per 6, 12 and 24 h groups and n=2 per control group) were perfused transcardially with heparinized saline, followed by 40 ml of chilled solution of 4% paraformaldehyde in 0.1 M phosphate buffer (pH 7.4). After postfixation in 4% paraformaldehyde for 4 h at 4° C.-6° C., brain was transferred into the cryoprotection solution of 30% ethylene glycol and 20% sucrose in 0.1 M phosphate buffer (pH 7.4) at 4° C. for 2-3 days. Tissue was then frozen on crushed dry ice and cut serially in 35 μm thick horizontal sections using a sliding microtome (Microm 400, Carl Zeiss). Section from different ischemic time points were stained under identical conditions using same reagents and solutions. For all immunocytochemical and histochemical protocols horizontally cut specimens were used.

Immunoreactive BVR was detected using rat polyclonal antibody raised in New Zealand white rabbit (S. D. Aust, Toxicol. Lett. 82/83:941-944 (1995); Huange et al., J. Biol. Chem. 264:7844-7849 (1989), which are hereby incorporated by reference). After 60 min of blocking in a solution of 5% normal goat serum in TBS followed by a wash in 0.25% Triton X-100 solution in TBS, all specimens were transferred into primary anti-BVR antibody that had been diluted 1:5000 in carrier solution (0.1 M TBS containing 0.5% goat serum, 0.25% Triton X-100), and incubated for 24 h at 4° C-6° C. (in Costar net wells). The specimens were then rinsed 5×10 min with 0.1 M TBS containing 0.25% Triton and placed into biotinylated secondary antibody reagent according to manufacturer's recommendations (Vectastain Elite, rabbit IgG kit). For peroxidase reactions, the typical incubation was 3 h at room temperature. Following 5×10 min washes in 0.1 M TBS, specimens were incubated at room temperature for 90 min in the avidin-biotin reagent prepared in 0.1 M PBS (ABC solution, Vector Labs, Burlingame, Calif.). After consecutive 10 min rinses in TBS and Tris-HCl, the sections were placed for 4-5 min into a filtered solution of 0.04% 3′,3′-diaminobenzidine (DAB), and 0.06% H₂O₂ in 0.1 M Tris buffer. Selected BVR immunolabeled specimens were double-stained with thionin—a histochemical neuronal nuclear labeling marker (Alvarez et al., Anat. Rec. 251:431-438 (1998), which is hereby incorporated by reference). Sections were dehydrated serially in 95% and 100% alcohols, incubated in histological grade xylene, mounted on Superfrost coated slides, and coverslipped with Permount (Fisher Scientific).

Histochemical Detection of Iron (III) and Lipid Peroxidation:

Iron (III) was detected by Perl's reaction followed by DAB enhancement (Hill et al., Neuroscience 11:595-603 (1984); Smith et al., Proc. Natl. Acad. Sci. USA 94:9866-9868 (1997), which are hereby incorporated by reference). Perl's reaction is based on the formation of ferric ferrocyanide (Prussian Blue) when ferric ion, released from iron-containing compounds by HCl, reacts with potassium ferrocyanide. The ferric ferrocyanide then catalyzes the oxidation of DAB with formation of a brown precipitate.

Lipid peroxidation at tissue level was assessed according to the method of Pompella et al. (Am. J. Pathol. 129:295-301 (1987), which is hereby incorporated by reference) based on detection of free aldehyde and carbonyl functions formed after peroxidative breakdown of unsaturated fatty acids. Free floating sections of mouse brains were incubated for 45 min in the dark at room temperature in Schiff's reagent (filtered pararosaniline base: thionyl chloride) prepared according to Barger and DeLamater as described by Pearse (Histochemistry. Theoretical and Applied, Vol. 1, 4^{th} edn., Churchill Livingstone, p. 655 (1980), which is hereby incorporated by reference). The sections were rinsed in three changes of sulfide water (1: 1, 10% K₂S₂O₅: 1 N HCl) before mounting. Histochemical staining for lipid peroxidation is incompatible with tissue processing for immunocytochemical procedures, thus no double labeling studies were performed.

Northern Hybridization:

Ischemic brains of mice were sacrificed at 6 and 24 h after MCAo. At each time point four miche per group were used. The contralateral hemisphere was used as control (Krupinsky et al., Stroke 28:654-673 (1997); Van Lookeren Campagne et al., Neuroscience 84:1097-1112 (1998), which are hereby incorporated by reference). For these analyses the cerebellum was not included. Total RNA was isolated from tissue and used for selection of poly(A+) RNA utilizing oligo (dT) cellulose chromatography and fractionated as before (Ewing et al., Brain Res. 672:29-41 (1995), which is hereby incorporated by reference). Hybridization probe was a PCR product consisting of nt+401 to 926 of BVR cDNA (Ewing et al., Brain Res. 672:29-41 (1995); Fakhrai et al., J. Biol. Chem. 267:4023-4029 (1992), which are hereby incorporated by reference), labeled using ³²P-dCTP with the Rediprime random primer labeling kit (Amersham, Arlington Heights, Ill.) following the manufacturer's instructions. The same method was used for labeling α-actin, which was used for loading control. Northern blots were quantified using BioRad model GS-700 imaging densitometer and Molecular Analyst v.1.5 software.

In situ Hybridization for Detection of BVR mRNA:

Immunochemical detection of digoxigenin-labeled BVR cDNA:mRNA hybrids was performed using oligonucleotide probes for BVR as described for HO-2 mRNA (Ewing et al., Brain Res. Protoc. 1:165-174 (1997), which is hereby incorporated by reference). Eight-micrometer specimens of control mice (n=2) of focal ischemic injury were used. BVR sense and antisense primers were from Midland Certified Reagent (Midland, Tex.). The sequence of the antisense oligonucleotide probe (SEQ. ID. No. 32) was as follows:

| | | |
|---|---|---|
| CTTCCTCCAG | GGACCCAG | 18 |

which is complimentary to nt+718→701 of rat kidney BVR (SEQ. ID. No. 4). The sequence of the sense oligonucleotide probe (SEQ. ID. No.33) was as follows:

| | | |
|---|---|---|
| TGCTCTCCGA | AGCCAAGAG | 19 |

which is complimentary to nucleotides +180→199 of rat kidney BVR (SEQ. ID. No. 4).
Western Blot Analysis and BVR Enzyme Activity:

Brain ischemic hemisphere after 6, 12 and 24 h after permanent MCAo and corresponding hemisphere of normal mice was used for preparation of cytosol. Samples used for Western blot analysis were subjected to SDS polyacrylamide gel electrophoresis under denaturing conditions. Western blot analysis was carried out as previously described (Huang et al., J. Biol. Chem. 264:7844-7849 (1989), which is hereby incorporated by reference). At each time point, tissue from three or four mice were pooled. BVR activity was measured using 1 mM NADH at pH 6.7 (Kutty et al., J. Biol. Chem. 256:3956-3962 (1981), which is hereby incorporated by reference). Assay was initiated by addition of cofactor (NADH). Conversion of biliverdin to bilirubin was protocoled as an increase in 450 nm absorbance at 30° C.

Statistical Analysis:

Intergroup statistical comparisons were performed using analysis of variance (ANOVA) followed by Scheffe's post-hoc analysis. Coefficient of variation was calculated as the ratio of the standard deviation to the mean multiplied by 100. Statistical comparisons were carried out using Statview v. 5.0 (SAS Institute, Cary, N.C.). Values of p<0.05 wer considered as statistically significant. Statistical analysis of results of intergroup behavioral assessment was performed using nonparametric Kruskal-Wallis test. The number of animals essential to prove intergroup significance is presented based on power analysis calculations (Primer of Biostatistics, v. 3.01, McGraw Hill).

Results and Discussion

Time-dependent Effects of Permanent MCA Occlusion on the Size of Ischemic Neuronal Injury and Animal Behavior:

Six hours after induction of ischemic neuronal injury by permanent occlusion of the MCA at the upper level of the olfactory tract, mice developed lesions that were 55±5 mm³ in size (FIG. 16A; coefficient of variation<10%; n=8). The size of the ischemic lesions increased to 63+6.7 mm³ at 12 h due to progressive involvement of the ipsilateral cortex and caudate nucleus (coefficient of variation <11%; n=8). By 24 h, the mean size of the ischemic lesions rose to 73±5 mm³ (coefficient of variation<7%; n=7). Based on this data, a time-dependent trend towards delayed maturation of the ischemic lesions during the first 24 h post MCAo was statistically confirmed (ANOVA: p=0.001; F=19; α=0.05 and power −0.95). This observation was further verified using Scheffe's post hoc analysis, which demonstrated significance at the p<0.05 level for each of the studied groups.

Upon occlusion of the MCA, mice developed Grade 2 behavioral deficits, and exhibited short radius circling. All animals made uneventful recovery and resumed normal grooming and feeding activity as early as 3-4 h after surgery. A time-dependent change in behavior did not parallel maturation of the ischemic lesions over the course of 24 h.

Intra- and Peri-ischemic BVR Immunocytochemistry During Brain Lesion Progression:

The overall pattern of BVR staining in normal and ischemic hemisphere at 24 h after MCAo is shown in FIGS. 16B-C. As noted, when compared with normal tissue (FIG. 16B), at 24 h (FIG. 16C), there is a marked increased BVR immunoreactivity in the area adjacent to the peri-ischemic lesion both in the cortex (arrowhead) and caudate nucleus (arrows). The contours of the ischemic lesions at 6 h were poorly discernible, but borders of the lesions became increasingly well defined by 12 h.

Persistent Expression of BVR Immunolabeling Within Ischemic Caudate Nucleus:

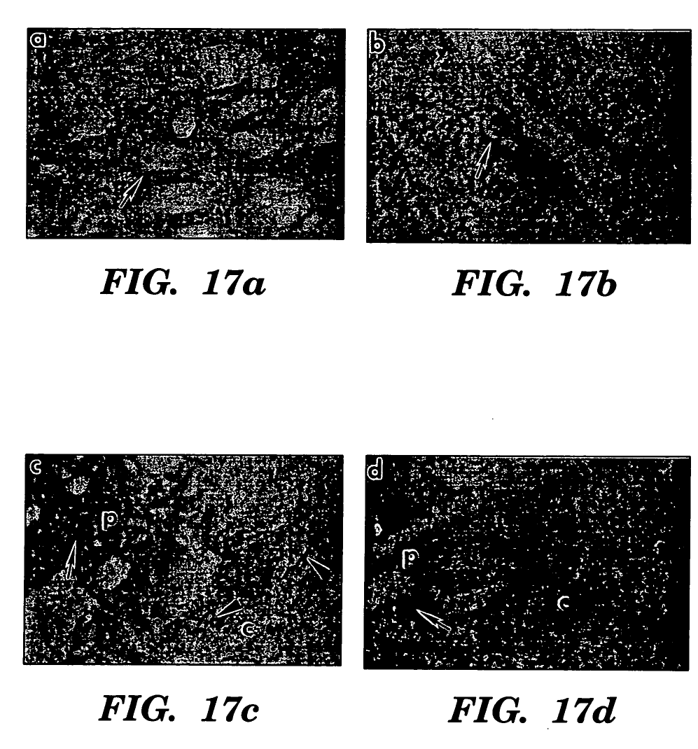

A closer examination of BVR in ischemic lesion in the caudate is shown in FIGS. 17A-D. Under normal conditions, BVR immunolabeling was present in select neurons in the caudate nucleus (FIG. 17A). Six hours after MCAo, prominent increase in BVR immunoreactivity in this region was observed (FIG. 17B). At 12 h, the number of BVR positive neurons and their labeling intensity were decreased in the ischemic core, but dramatically increased in the ischemic penumbra (FIG. 17C) and in adjacent peri-ischemic territories. A few BVR-labeled neurons were primarily detected in the vicinity of capillary branches of the LSA (branch of MCA), which demonstrated loss of vascular arborizations. At 24 h post MCAo (FIG. 17D), the increased neuronal immunolabeling for BVR persisted in the ischemic penumbra and peri-ischemic regions.

BVR Expression Correlates with Neuronal Cell Survival in Cortical Layers 3 and 5:

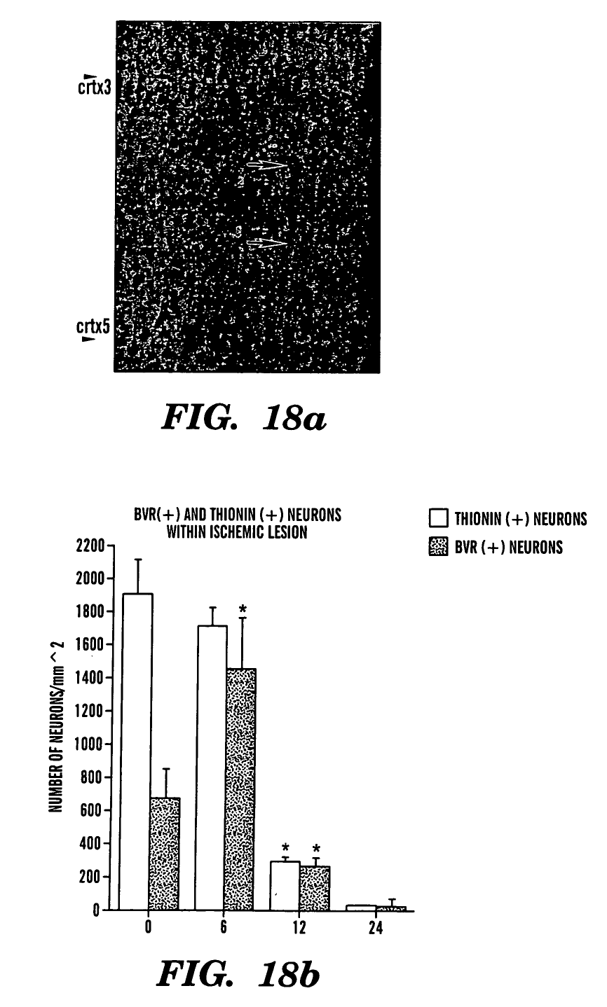

In control mice, neuronal labeling for BVR was observed in the cortical region of the forebrain, diencephalic and brainstem regions, as well as in the cerebellum. This finding is in agreement with a previous report (Ewing et al., Brain Res. 672:29-41 (1995), which is hereby incorporated by reference). Six hours after MCAo, the cortical area of the ischemic lesion exhibited loss of BVR immunoreactive neurons in all layers other than layers 3 and 5 (FIG. 18A). This observation was confirmed by double staining for BVR and thionin (FIG. 18B) and iron (III) staining of the specimens. Double labeling of specimens for BVR and thionin confirmed that the majority of surviving neurons in layers 3 and 5 were BVR positive. As noted, the ratio of labeling for BVR and thionin was nearly 1:1 at 6, 12 and 24 h after MCAo. Specifically, the number of BVR positive cortical neurons 6 h after MCAo was increased by two-fold in the ischemic lesion area (n=4; 1451±31 ; p <0.01) when compared with neuronal cell counts from corresponding 0 time point sections (n=4; 664±185). By 12 h, the number of double-labeled neurons progressively decreased to 263+50 (n=4) and only one of four mice at 24 h demonstrated presence of few double stained neurons in the periphery of the ischemic core.

Expression of BVR in Non-ischemic Areas of the Brain:

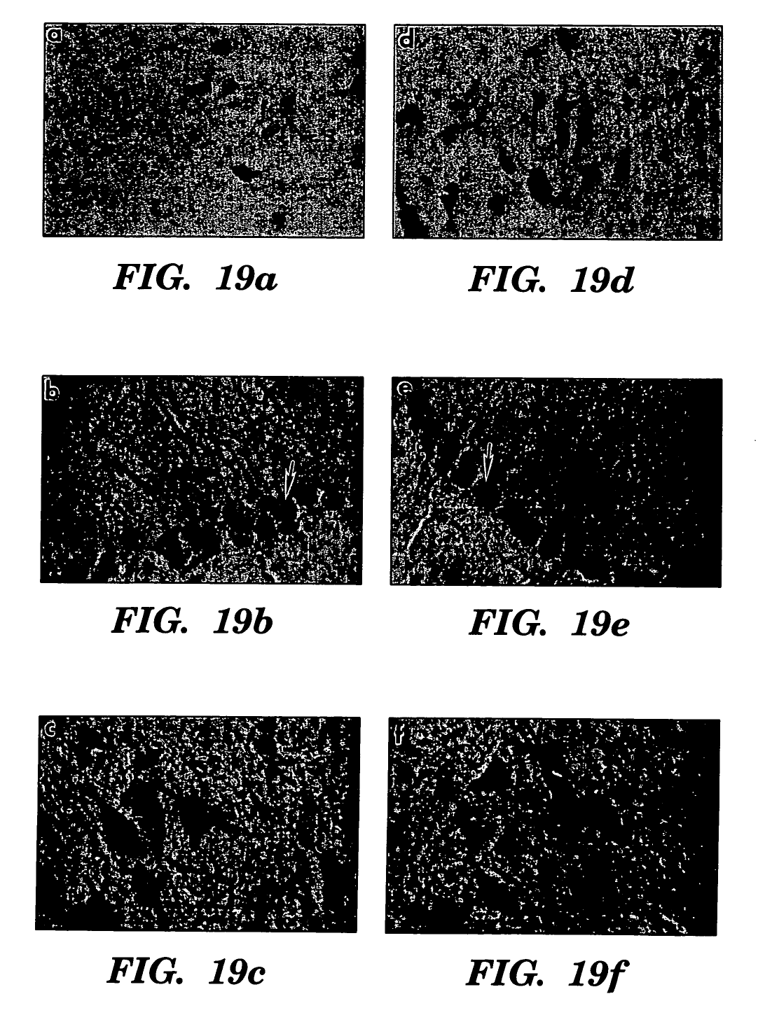

Postischemic changes in BVR immunoreactivity were not just restricted to local ipsilateral (ischemic) areas of cortex and caudate nucleus, but were also found in distant areas of the brainstem and cerebellum. For example, as shown in FIGS. 19A-F, when compared with control specimens (FIGS. 19A-C), 6 h after MCAo (FIGS. 19D-F) neurons of substantia nigra (FIG. 19A versus 19D), Purkinje neurons of the cerebellum (FIG. 19B versus 19E) and neurons of the central nucleus of inferior colliculus (CIC; FIG. 19C versus 19F) show an overall increase in antibody staining for the reductase. Noteworthy is the discrete and prominent nuclear staining of neuronal cells in CIC region 6 h after MCAo (FIG. 19F).

Increase in Lipid Peroxidation at the Margin of Ischemic Penumbra:

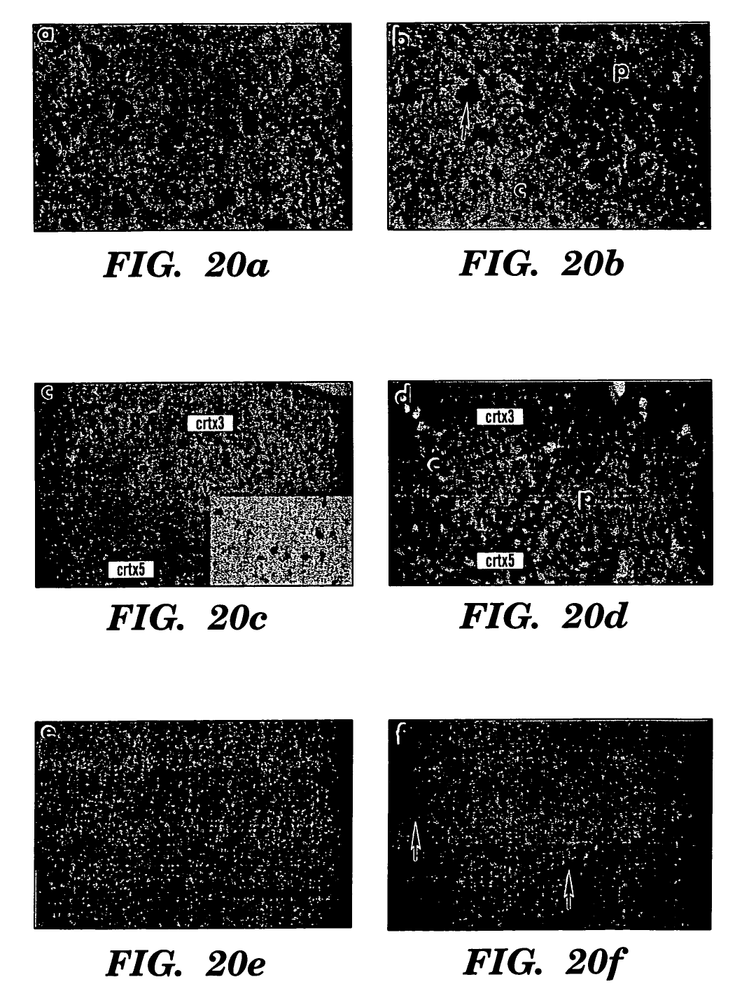

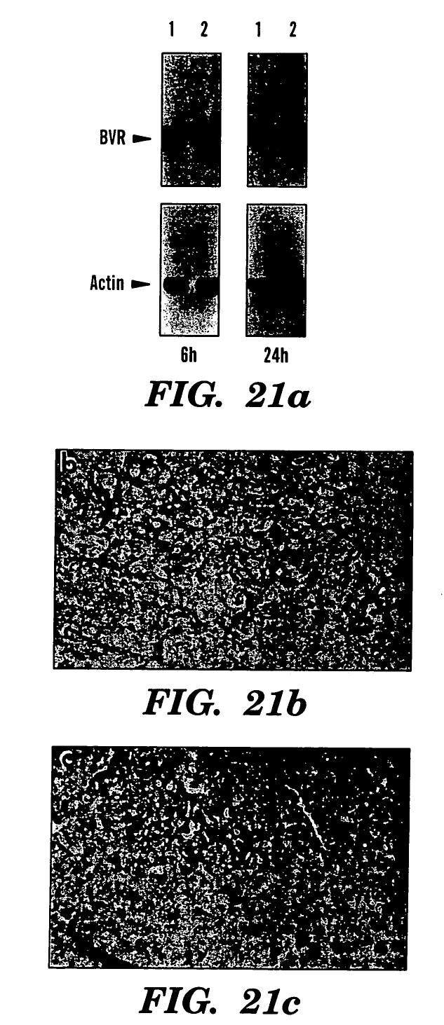

In the ischemic cortex (FIGS. 20A-F), as with the ischemic caudate (FIGS. 17A-D), expression of BVR persisted throughout the duration of experiment (6, 12 and 24 h after MCAo vs. control) within the peri-ischemic region. The results of findings with 6 and 24 h of MCAo treatment are shown in FIGS. 20A-F. At this time, a marked increase in the BVR immunoreactivity of neuronal cell bodies and processes in peri-ischemic areas was noted (FIG. 20A). In the same cortical region of the control brain tissue, only select neurons expressed BVR immunoreactivity. Intense immunolabeling for BVR in neuronal cell bodies was also noted in cortical peri-ischemic regions of mice subjected to 12 h after MCAo, however, most BVR positive neurons in the vicinity of the ischemic lesion lacked cellular arborizations. At 24 h, neurons located at the immediate border with the ischemic core demonstrated intense BVR immunoreactivity, as well as loss of cellular processes (FIG. 20B).

Tissue staining for iron was used as the index of heme degradation activity. Neuronal labeling for iron (III) 6 h after MCAo in cortical layers 3 and 5 are shown in FIG. 20C. As shown, a prominent staining of neuronal cell bodies is detectable in both layers, layer 3 neurons, however, were more prominently labeled when compared with those in layer 5. Such pattern of labeling was not observed in control specimens, in which only select neurons displayed iron histochemical staining. Labeling with iron of cortical microvessels as well as presence of background labeling were also observed 12 h after MCAo. At 24 h after ischemia (FIG. 20D), prominent staining for iron was present throughout the area of the ischemic core and penumbral areas. The area of iron staining, however, was less than that of BVR immunostaining.

Staining with Schiff's reagent was used for detection of lipid peroxidation. Under normal conditions brain specimens did not display Schiff's staining, labeling was also not observed in the contralateral hemisphere at 6, 12 or 24 h time points after MCAo. The development of lipid peroxidation in the ischemic hemisphere at 6 and 24 h after MCAo is shown in FIGS. 20E-F, respectively. As noted, lipid peroxidation activity is minimally detectable at 6 h post MCAo (FIG. 20E). At 24 (FIG. 20F) time point, however, a positive Schiff staining rim of cells, up to 95 μm wide, circumscribed the entire core of the ischemic lesion.

Measurements of the Levels of BVR mRNA, Protein, and Activity:

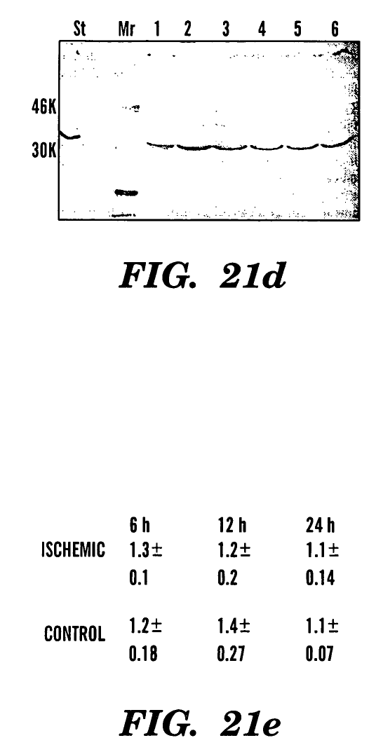

In situ hybridization, Northern and Western blot analyses were carried out to examine the time-dependent effect of MCAo on expression of BVR message and protein in the ischemic hemisphere. Comparisons were made to those parameters measured in the contralateral hemisphere as well as intact control brain (for in situ hybridization). As shown in FIG. 21 A, Northern blot analysis of brain hemispheres obtained at 6 and 24 h after MCAo did not demonstrate a change in BVR mRNA levels. In contrast, marked local increases in BVR mRNA were detected by in situ hybridization in the penumbral and adjacent peri-ischemic regions both at time points post MCAo (FIGS. 21B-C). As with Northern blot analysis, Western blot analysis (FIG. 21D) showed that discernible differences in total BVR were not detected between the contralateral (lanes 1, 3, 5) and ischemic hemispheres (lanes 2, 4, 6) at 6, 12 and 24 h after MCAo despite the total increase in BVR in immunostaining in the peri-ischemic region (FIGS. 17C-D). The absence of notable changes in total BVR mRNA and protein with time suggest that the overall capacity of the tissue to generate biliverdin is tightly controlled. This suggestion is supported by the finding that enzyme activity of the impaired hemisphere also did not change over the course of 24 h (FIG. 21E).

The presently observed intense neuronal BVR staining in the perimeter of ischemic lesion can be interpreted as either (i) increased BVR expression defining a “line of injury” or (ii) increased BVR expression defining a “line of defense” against advancement of ischemic injury. The increased expression of BVR at the perimeter of ischemic damage and peri-ischemic areas is not unique to this protein and has been observed with a number of other proteins with very diverse physiological function, including iNOS (Galea et al., Am. J. Physiol. 274:H2035-H2045 (1998), which is hereby incorporated by reference), caspases (Maiese et al., Neurosci. Lett. 264:17-20 (1999); Willis et al., Nat. Med. 2:87-90 (1996), which are hereby incorporated by reference), polyamine oxidase (Ivanova et al., J. Exp. Med. 188:327-340 (1998), which is hereby incorporated by reference), platelet-derived growth factor (Krupinsky et al., Stroke 28:654-673 (1997), which is hereby incorporated by reference), trkA proteins (Lee et al., Stroke 29:1687-1697 (1998), which is hereby incorporated by reference), hepatocyte growth factor (Hayashi et al., Brain Res. 799:311-316 (1998), which is hereby incorporated by reference), ciliary neurotrophic factor (CNTF) (Lin et al., Mol. Brain Res. 55:71-80 (1998), which is hereby incorporated by reference), genes in charge of control of the cell cycle such as cyclin G1 and p21 (Van Lookeren Campagne et al., Neuroscience 84:1097-1112 (1998), which is hereby incorporated by reference), as well as early response genes (Kinouchi et al., NeuroReport 10:1055-1059 (1999); Kinouchi et al., J. Cereb. Blood Flow Metab. 13:105-115 (1993); Lee et al., Stroke 29:1687-1697 (1998), which are hereby incorporated by reference), and HO-1 itself (Geddes et al., Neurosci. Lett. 210:205-208 (1996), which is hereby incorporated by reference). Based on the following reasonings, however, it is believed that the “line of defense” theory is most probable.

The previous findings that BVR levels are increased in those neuronal populations that display high level expression of the stress inducible form of heme oxygenase (HO-1 or HSP32) under ischemic conditions (Bergeron et al., J. Cereb. Blood Flow Metab. 17:647-658 (1997); Geddes et al., Neurosci. Lett. 210:205-208 (1996); Nimura et al., Mol. Brain Res. 37:201-208 (1996); Takizawa et al., J. Cereb. Blood Flow Metab. 18:559-569 (1998), which are hereby incorporated by reference), and the commonly accepted role of heat shock proteins in cellular defense mechanisms (Kinouchi et al., J. Cereb. Blood Flow Metab. 13:105-115 (1993), which is hereby incorporated by reference) are consistent with the above suggestions. In this capacity, BVR would accelerate reduction of biliverdin, production of which is increased as indicated by enhanced staining for iron within the perimeter of ischemic injury. The only source for iron in brain is the heme molecule (Maines, Heme Oxygenase: Clinical Applications and Functions, CRC Press, Boca Raton, Fla. (1992), which is hereby incorporated by reference). Iron is a known catalyst for generation of oxygen free radicals. One consequence of their generation, when not opposed, is peroxidation of membrane lipids. As noted here, although iron staining of tissue in the perimeter of ischemic injury is increased, staining for lipid peroxidation is observed only in the margin of the ischemic lesion. This observation can be interpreted as an indication that, at the border of the lesion, the catalytic activity of iron to generate free radicals has prevailed over the antioxidant activity of bile pigments. This suggestion is in agreement with the finding that the volume of BVR staining in the perimeter of ischemic injury exceeded that of iron staining.

Other observations made in this study are consistent with the above model. For instance, intense BVR staining of pyramidal neurons and their processes in cortical layers 3 and 5 within the epicenter of ischemic injury at 6 h post MCAo may also be relevant. Neurons of these cortical layers are known to be selectively vulnerable to conditions of transient global cerebral ischemia (Akulnin et al., Resuscitation 35:157-164 (1997); Kaplin et al., J. Neurosci. 16:2002-2011 (1996), which are hereby incorporated by reference), but are also known to express a number of growth factors after focal cerebral ischemia (Hayashi et al., Brain Res. 799:311-316 (1998); Lee et al., Stroke 29:1687-1697 (1998); Lin et al., Mol. Brain Res. 55:71-80 (1998), which are hereby incorporated by reference). The possibility, however, cannot be dismissed that the increase in BVR expression in these neurons predisposes the cells to damage. In addition, increased immunolabeling for BVR after 6 h of MCAo was also observed in areas of the brain distant from the site of ischemic insult. This included Purkinje neurons of the cerebellum, which are vulnerable to an array of toxic agents (Smeyne et al., Mol. Cell. Neurosci. 6:230-251 (1995), which is hereby incorporated by reference) and hypoxic conditions (Krajewski et al., J. Neurosci. 15:6364-6376 (1995), which is hereby incorporated by reference); the substantia nigra, which is the center for dopaminergic activity, is compromised in Parkinson's disease, and is susceptible to iron related toxicities (Jenner et al., Ann. Neurol. 44 (3):72-84, Suppl. 1 (1998); Lin et al., Ann. N. Y. Acad. Sci. 825:134-145 (1997); Simonian et al., Annu. Rev. Pharmacol. Toxicol. 36:83-106 (1996), which are hereby incorporated by reference); and the central nucleus of inferior colliculus, which is an important synaptic relay in the central auditory pathway (Higashiyma et al., Exp. Neurol. 153:94-101 (1998), which is hereby incorporated by reference). The observed increases in distant cell populations may suggest a cellular response to accommodate cellular demand for conversion of biliverdin to bilirubin brought about by increased heme degradation by HO isozymes. HO-1 is highly responsive to oxidative stress in these exact neuronal populations (Ewing et al., J. Neurochem. 61:1015-1023 (1993); Panahian et al., J. Neurochem. 72:1187-1203 (1999), which are hereby incorporated by reference). It is noteworthy that increased gene expression in areas remote from ischemic injury is not specific for BVR and has been reported for several immediate early genes (Kinouchi et al., NeuroReport 10:1055-1059 (1999), which is hereby incorporated by reference). A possible mechanism for the regulation of gene expression in distant areas has been postulated to relate to transsynaptic activation.

Under conditions of ischemia (Hicks et al., Gen. Pharmacol. 30:265-273 (1998), which is hereby incorporated by reference) and stress, impairment of neuronal ribosomal protein synthesis takes place (Hu et al., J. Neurosci. 13:1830-1838 (1993), which is hereby incorporated by reference). As the lesion matures, proteins present within the ischemic core may become denatured as a consequence of cell injury (Kinouchi et al., J. Cereb. Blood Flow Metab. 13:105-115 (1993); Richmon et al., Brain Res. 780:108-118 (1998), which are hereby incorporated by reference). In this context, the present finding of the unchanged levels of BVR mRNA and total protein, as assessed by Northern and Western blot analyses, respectively, and activity in the ischemic hemisphere with progression of ischemia suggest a tightly regulated mechanism for BVR gene expression. Furthermore, the absence of a difference between the ipsilateral and the contralateral hemispheres in these parameters may suggest the upregulation of BVR gene expression in the peri-ischemic region compensates for the loss of BVR in the ischemic core. The seemingly tightly controlled regulating mechanisms would prevent production of neurotoxic levels of bilirubin. It is noteworthy that kemicterus is caused by high levels of circulating bilirubin giving access to the brain tissue (Maines, Heme Oxygenase: Clinical Applications and Functions, CRC Press, Boca Raton, Fla. (1992), which is hereby incorporated by reference).

Aside from the known antioxidant activity, based on the unique catalytic properties of the enzyme with respect to its dual pH/cofactor requirements (Huange et al., J. Biol. Chem. 264:7844-7849 (1989); Kutty et al., J. Biol. Chem. 256:3956-3962 (1981), which are hereby incorporated by reference), additional function(s) in the cell can be reasonably ascribed to the reductase, one being its dehydrogenation of NADH. This function may contribute to the reported increased utilization and lowering NADH levels that occur in the ischemic neurons. The decrease in cellular NADH could lower capacity of the cell to mediate NADH-driven iron-mediated Fenton reaction, thus blocking production of free radicals. This activity of BVR is analogous to that of NADH-dehydrogenase, induction of which has been suggested to result in decreased levels of NADH and reduced levels of cellular redox cycling constituents (Stadtman et al., Drug Metab. Rev. 30:225-243 (1998), which is hereby incorporated by reference). Furthermore, increased utilization of NADH causes depletion of ATP (Samdani et al., Stroke 28:1283-1288 (1997), which is hereby incorporated by reference), which is used as a cofactor by poly (ADP-ribose) polymerase (PARP). Activation of this polymerase leads to death (Eliasson et al., Nat. Med. 3:1089-1095 (1997); Endres et al., J. Cereb. Blood Flow Metab. 17:1143-1151 (1998); Lo et al., Stroke 29:830-836 (1998), which are hereby incorporated by reference) in damaged cells. Furthermore, during cerebral ischemia, there is a substantial drop in neuronal pH to 6.7 within the first 30 min following MCAo (Regli et al., J. Cereb. Blood Flow Metab. 16:988-995 (1996), which is hereby incorporated by reference). Indeed, lower pH values of 5.3 to 5.9 are attained by astrocytes throughout the course of ischemic injury (Kraig et al., J. Cereb. Blood Flow Metab. 10:104-114 (1990), which is hereby incorporated by reference), and BVR can effectively function in this pH range (Fakhrai et al., J. Biol. Chem. 267:4023-4029 (1992); Huang et al., J. Biol. Chem. 264:7844-7849 (1989); Maines et al., Arch. Biochem. Biophys. 300:320-326 (1993), which are hereby incorporated by reference).

Thus, the present findings with BVR expression, on the balance, are supportive of a neuroprotective role of heme degradation products in cerebral ischemia and against reactive oxygen species, which have been implicated in neuronal cell death (Simonian et al., Annu. Rev. Pharmacol. Toxicol. 36:83-106 (1996), which is hereby incorporated by reference).

### Example 6—Effect of Anti-sense BVR Expression on Cell Survivability with and Without Presence of Toxins

Antisense BVR DNA plasmid construct was generated using a DNA fragment representing the 5′ end of the human BVR cDNA. The DNA fragment was generated by PCR using the following primers:

| | | | |
|---|---|---|---|
| GGCAAGCTTG | TGGCGCCCGG | AGCTGC | 25 |

(SEQ. ID. No. 36) which represents nucleotides −57 to −41 (Maines et al., Eur. J. Biochem. 235(1-2):372-381 (1996); Maines et al., Arch. Biochem. Biophys. 300(1):320-326 (1993), which are hereby incorporated by reference) and has a HindIII linker (underlined); and

| | | | |
|---|---|---|---|
| GGCAAGCTTC | ATCAATGCTC | CCGAGCTC | 28 |

(SEQ. ID. No. 37) which represents the reverse complement of the nucleotides +139 to +157 (Maines et al., Eur. J. Biochem. 235(1-2):372-381 (1996); Maines et al., Arch. Biochem. Biophys. 300(1):320-326 (1993), which are hereby incorporated by reference) and has a HindIII linker (underlined). The products were initially cloned into the PCR cloning vector PCR II and the sequence confirmed. The insert was then excised using HindIII, ligated into pcDNA3, digested with the same enzyme and subsequently transformed into E. coli XL-blue. The orientation of DNA fragments was determined by sequence analysis. Transformed E. coli cells were then grown in super broth containing ampicillin and the plasmid DNA isolated using bigger prep isolation kit (5 Prime-3 Prime Inc., Boulder, Co.).

After CsCl gradient purification, the construct was used to transfect COS cell by electroporation under standard conditions. Transfected COS cells were grown in DMEM medium plus increasing concentrations of geneticin (from 100-450 μm). The cells were then used to examine response to oxidative stress imposed by hemin, sodium arsenite, and menadione.

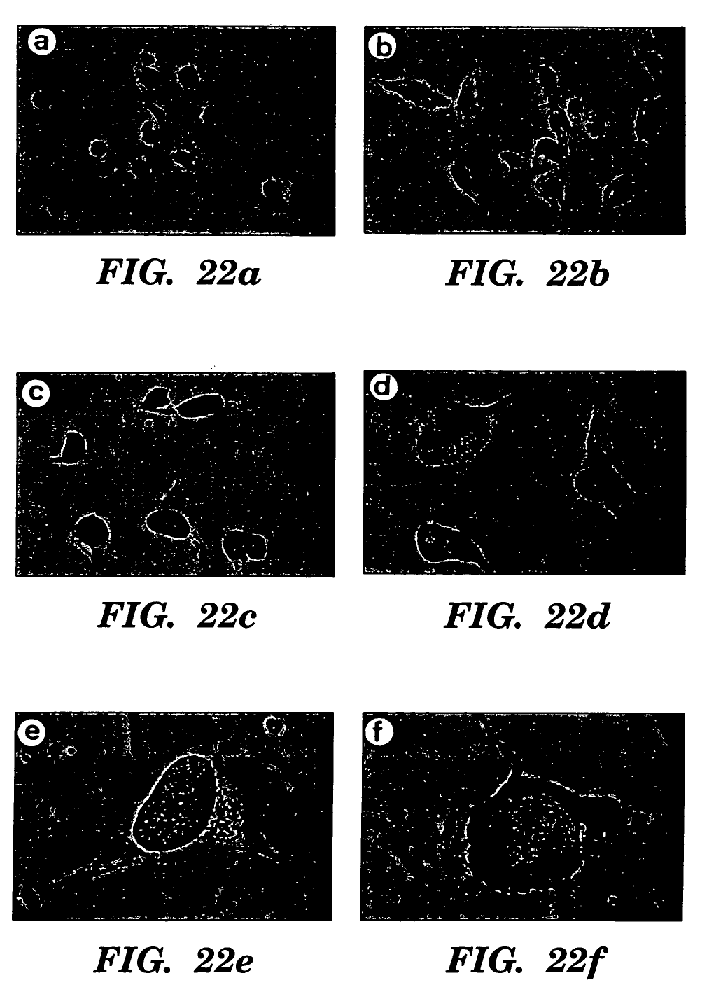

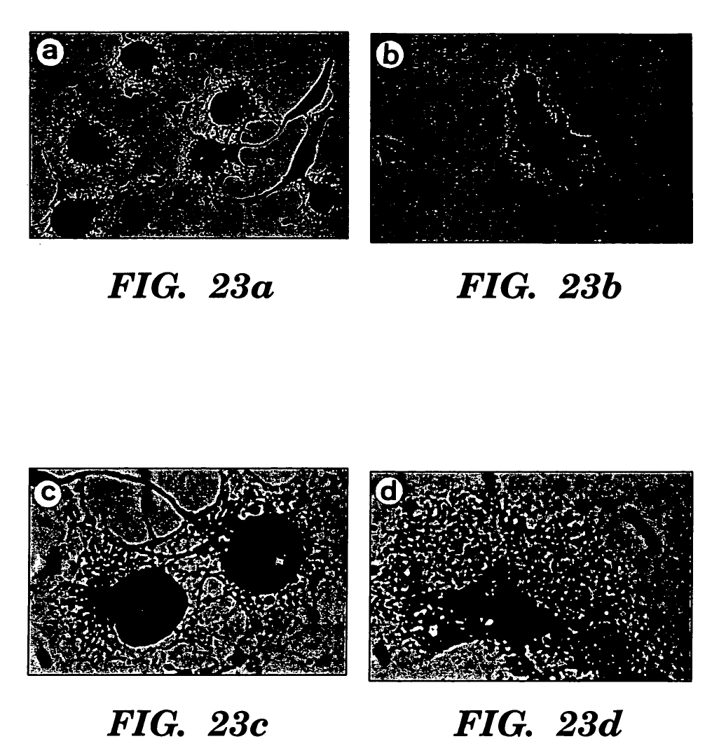

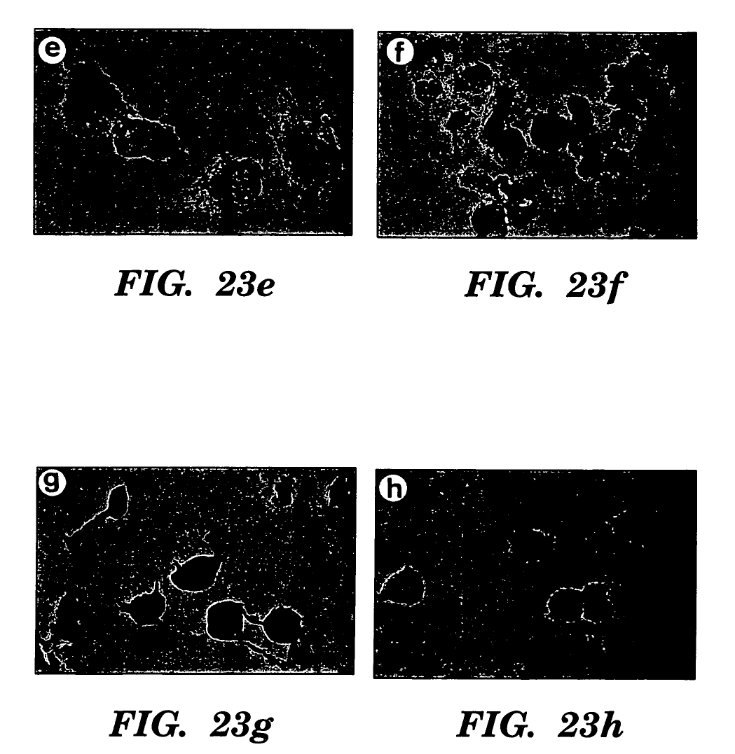

FIGS. 22A, C, and D show cellular morphology of control cells versus those of anti-sense treated cells. Three different magnifications of the cells are shown to demonstrate that those cells which have been treated with anti-sense BVR display compromised cellular morphology. FIGS. 22A-B, 10× magnification; FIGS. 22C-D, 40× magnification; and FIGS. 22E-F, 100× magnification. In addition, these cells appear to be stressed as indicated by increased staining for the stress protein heme oxygenase-1. FIGS. 23A-H illustrate the response of these two lines of cells to hematin (FIGS. 23A-D), sodium arsenite (FIGS. 23E-F), menadione (FIGS. 23G-H). FIGS. 23A, 23C, 23E, and 23G are control cells, whereas FIGS. 23B, 23D, 23F, and 23H are anti-sense transfected cells. As shown, the anti-sense cells are severely damaged by the treatments, particularly as noted in FIG. 23A versus 23B and FIG. 23C versus 23D, that cellular morphology is intact in control cells, whereas it is compromised in the anti-sense cells treated with hematin. Also, in the presence of anti-sense, the nuclear localization of heme oxygenase-1 is blocked, which suggests that biliverdin reductase is a carrier of messages from cytosol into the nucleus. FIGS. 23E-H also show the phenomena of severe injury in the presence of anti-sense BVR in cells treated with toxins arsenite and menadione.

Without being bound by theory, it is believed that the anti-sense BVR diminished the expression levels of BVR, thereby exposing the cells to oxidative stress and diminished ability to survive treatment with toxins.

Although the invention has been described in detail for the purposes of illustration, it is understood that such detail is solely for that purpose, and variations can be made therein by those skilled in the art without departing from the spirit and scope of the invention which is defined by the following claims.

1. A method of regulating protein kinase C activity comprising:
contacting human protein kinase C selected from the group of isozymes α, β, and γ with a mammalian biliverdin reductase or a fragment thereof with protein kinase C regulatory activity, wherein the mammalian biliverdin reductase is encoded by a nucleic acid molecule that hybridizes to the complement of SEQ ID NO: 2 or 5 under hybridization conditions comprising a temperature of 65° C. and a hybridization medium comprising 1 M Na⁺ buffer and remains hybridized following wash conditions comprising a temperature of 65° C. and a wash medium comprising 0.2×SSC buffer, and wherein the fragment thereof comprises the amino acid sequence of SEQ ID NO: 18, 19, 34, or 35, said contacting being effective to regulate activity of the human protein kinase C.

2. The method according to claim 1, wherein said contacting is carried out with rat or human biliverdin reductase.

3. The method according to claim 2, wherein the biliverdin reductase is human biliverdin reductase comprising an amino acid sequence according to SEQ ID NO: 1 or SEQ ID NO: 3.

4. The method according to claim 1, wherein said contacting is carried out with the fragment of the mammalian biliverdin reductase that comprises the amino acid sequence of SEQ ID NO: 18, 19, 34, or 35.

5. The method according to claim 4, wherein the fragment of the mammalian biliverdin reductase consists of the amino acid sequence of SEQ ID NO: 18, 19, 34, or 35.

6. The method according to claim 1, wherein said contacting is carried out in a cell.

7. The method according to claim 5, wherein the cell is in vivo.

8. The method according to claim 5, wherein the cell is in vitro.


SEQUENCE LISTING (37 sequences)

SEQ ID NO: 1 - protein, 296 aa, Homo sapiens
MNAEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTSDPLEEDRFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 2 - DNA, 1070 bp, Homo sapiens
ggggtggcgcccggagctgcacggagagcgtgcccgtcagtgaccgaagaagagaccaagatgaatgcagagcccgagaggaagtttggcgtggtggtggttggtgttggccgagccggctccgtgcggatgagggacttgcggaatccacacccttcctcagcgttcctgaacctgattggcttcgtgtcgagaagggagctcgggagcattgatggagtccagcagatttctttggaggatgctctttccagccaagaggtggaggtcgcctatatctgcagtgagagctccagccatgaggactacatcaggcagttccttaatgctggcaagcacgtccttgtggaataccccatgacactgtcattggcggccgctcaggaactgtgggagctggctgagcagaaaggaaaagtcttgcacgaggagcatgttgaactcttgatggaggaattcgctttcctgaaaaaagaagtggtggggaaagacctgctgaaagggtcgctcctcttcacatctgacccgttggaagaagaccggtttggcttccctgcattcagcggcatctctcgactgacctggctggtctccctctttggggagctttctcttgtgtctgccactttggaagagcgaaaggaagatcagtatatgaaaatgacagtgtgtctggagacagagaagaaaagtccactgtcatggattgaagaaaaaggacctggtctaaaacgaaacagatatttaagcttccatttcaagtctgggtccttggagaatgtgccaaatgtaggagtgaataagaacatatttctgaaagatcaaaatatatttgtccagaaactcttgggccagttctctgagaaggaactggctgctgaaaagaaacgcatcctgcactgcctggggcttgcagaagaaatccagaaatattgctgttcaaggaagtaagaggaggaggtgatgtagcacttccaagatggcaccagcatttggttcttctcaagagttgaccattatctctattcttaaaattaaacatgttggggaaacaaaaaaaaaaaaaaaaa

SEQ ID NO: 3 - protein, 296 aa, Homo sapiens
MNTEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 4 - protein, 295 aa, Rattus norvegicus
MDAEPKRKFGVVVVGVGRAGSVRLRDLKDPRSAAFLNLIGFVSRRELGSLDEVRQISLEDALRSQEIDVAYICSESSSHEDYIRQFLQAGKHVLVEYPMTLSFAAAQELWELAAQKGRVLHEEHVELLMEEFEFLRREVLGKELLKGSLRFTASPLEEERFGFPAFSGISRLTWLVSLFGELSLISATLEERKEDQYMKMTVQLETQNKGLLSWIEEKGPGLKRNRYVNFQFTSGSLEEVPSVGVNKNIFLKDQDIFVQKLLDQVSAEDLAAEKKRIMHCLGLASDIQKLCHQKK

SEQ ID NO: 5 - DNA, 1081 bp, Rattus norvegicus
ggtcaacagctaagtgaagccatatccatagagagtttgtgccagtgccccaagatcctgaacctctgtctgtcttcggacactgactgaagagaccgagatggatgccgagccaaagaggaaatttggagtggtagtggttggtgttggcagagctggctcggtgaggctgagggacttgaaggatccacgctctgcagcattcctgaacctgattggatttgtgtccagacgagagcttgggagccttgatgaagtacggcagatttctttggaagatgctctccgaagccaagagattgatgtcgcctatatttgcagtgagagttccagccatgaagactatatacggcagtttctgcaggctggcaagcatgtcctcgtggaataccccatgacactgtcatttgcggcggcccaggagctgtgggagctggccgcacagaaagggagagtcctgcatgaggagcacgtggaactcttgatggaggaattcgaattcctgagaagagaagtgttggggaaagagctactgaaagggtctcttcgcttcacagctagcccactggaagaagagagatttggcttccctgcgttcagcggcatttctcgcctgacctggctggtctccctcttcggggagctttctcttatttctgccaccttggaagagcgaaaagaggatcagtatatgaaaatgaccgtgcagctggagacccagaacaagggtctgctgtcatggattgaagagaaagggcctggcttaaaaagaaacagatatgtaaacttccagttcacttctgggtccctggaggaagtgccaagtgtaggggtcaataagaacattttcctgaaagatcaggatatatttgttcagaagctcttagaccaggtctctgcagaggacctggctgctgagaagaagcgcatcatgcattgcctggggctggccagcgacatccagaagctttgccaccagaagaagtgaagaggaagcttcagagacttctgaagggggccagggtttggtcctatcaaccattcacctttagctcttacaattaaacatgtcagataaaca

SEQ ID NO: 6 - protein, 6 aa, Artificial Sequence
  Description of Artificial Sequence hydrophobic domain of BVR
FXVVVV

SEQ ID NO: 7 - protein, 6 aa, Artificial Sequence
  Description of Artificial Sequence  nucleotide binding domain of BVR
GXGXXG

SEQ ID NO: 8 - protein, 8 aa, Artificial Sequence
  Description of Artificial Sequence oxidoreductase domain of BVR
AGKHVLVE

SEQ ID NO: 9 - protein, 29 aa, Artificial Sequence
  Description of Artificial Sequence  leucine zipper of BVR
LXXXXXXLXXXXXXKXXXXXXLXXXXXXL

SEQ ID NO: 10 - protein, 3 aa, Artificial Sequence
  Description of Artificial Sequence  kinase motif of BVR
SRR

SEQ ID NO: 11 - protein, 3 aa, Artificial Sequence
  Description of Artificial Sequence  kinase motif of BVR
KGS

SEQ ID NO: 12 - protein, 3 aa, Artificial Sequence
  Description of Artificial Sequence  kinase motif of BVR
FGX

SEQ ID NO: 13 - protein, 7 aa, Artificial Sequence
  Description of Artificial Sequence  nuclear localization signal of BVR
GLKRNRY

SEQ ID NO: 14 - protein, 5 aa, Artificial Sequence
  Description of Artificial Sequence methylation site of BVR
PGLKR

SEQ ID NO: 15 - protein, 14 aa, Artificial Sequence
  Description of Artificial Sequence  zinc finger domain of BVR
HCXXXXXXXXXXCC

SEQ ID NO: 16 - protein, 7 aa, Artificial Sequence
  Description of Artificial Sequence  protein kinase C enhancing domain
KKRIXHC

SEQ ID NO: 17 - protein, 8 aa, Artificial Sequence
  Description of Artificial Sequence  protein kinase C inhibiting domain
QKXCXXXK

SEQ ID NO: 18 - protein, 7 aa, Artificial Sequence
  Description of Artificial Sequence  protein kinase C enhancer peptide of rBVR
KKRIMHC

SEQ ID NO: 19 - protein, 8 aa, Artificial Sequence
  Description of Artificial Sequence  protein kinase C inhibitor peptide of rBVR
QKLCHQKK

SEQ ID NO: 20 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 21 - DNA, 3660 bp, Homo sapiens
aatctatcagggaacggcggtggccggtgcggcgtgttcggtgcgctctggccgctcaggccgtgcggctgggtgagcgcacgcgaggcggcgaggcggcaagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtggtaattgggagaggtagccgagtcacacccggtggctgtggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaaagcttcgttagaatgtctgccttactggtttccccagggaaggaaaaatacacttccacccttttttctaagtgttcgtctttagttttgattttggaaagatgttaagcatttatttttagttaaaataaaaactaatttcatact

SEQ ID NO: 22 - DNA, 25 bp, Artificial Sequence
  Description of Artificial Sequence  primer
ggtcgacgaatgcagagcccgagag

SEQ ID NO: 23 - DNA, 27 bp, Artificial Sequence
  Description of Artificial Sequence  primer
gggcgaattcgtcgacttacttccttg

SEQ ID NO: 24 - DNA, 43 bp, Artificial Sequence
  Description of Artificial Sequence  primer
ggaagcttaaatatcctgtggatcctataacaggtcctttttc

SEQ ID NO: 25 - protein, 6 aa, Artificial Sequence
  Description of Artificial Sequence  mutant nuclear translocation signal
VIGSTG

SEQ ID NO: 26 - DNA, 56 bp, Artificial Sequence
  Description of Artificial Sequence  primer
gggatccatgtacccctacgacgtgcccgactacgccaatgcagagcccgagagga

SEQ ID NO: 27 - DNA, 27 bp, Artificial Sequence
  Description of Artificial Sequence  primer
gctcgagctcctcctcttacttccttg

SEQ ID NO: 28 - DNA, 57 bp, Artificial Sequence
  Description of Artificial Sequence  primer
gctcgagatgtacccctacgacgtgcccgactacgccatgacagtgtgtctggagac

SEQ ID NO: 29 - protein, 8 aa, Artificial Sequence
  Description of Artificial Sequence  protein kinase C inhibitor
RKRCLRRL

SEQ ID NO: 30 - DNA, 29 bp, Artificial Sequence
  Description of Artificial Sequence  primer
ggtcgacagagaccgagttggatgccgag

SEQ ID NO: 31 - DNA, 29 bp, Artificial Sequence
  Description of Artificial Sequence  primer
gcggccgtcgtctctgaatcttcctcttc

SEQ ID NO: 32 - DNA, 18 bp, Artificial Sequence
  Description of Artificial Sequence  probe
cttcctccagggacccag

SEQ ID NO: 33 - DNA, 19 bp, Artificial Sequence
  Description of Artificial Sequence  probe
tgctctccgaagccaagag

SEQ ID NO: 34 - protein, 7 aa, Artificial Sequence
  Description of Artificial Sequence  protein kinase C enhancer peptide of hBVR
KKRILHC

SEQ ID NO: 35 - protein, 8 aa, Artificial Sequence
  Description of Artificial Sequence  protein kinase C inhibitor peptide of hBVR
QKYCCSRK

SEQ ID NO: 36 - DNA, 26 bp, Artificial Sequence
  Description of Artificial Sequence  primer
ggcaagcttgtggcgcccggagctgc

SEQ ID NO: 37 - DNA, 28 bp, Artificial Sequence
  Description of Artificial Sequence  primer
ggcaagcttcatcaatgctcccgagctc